SEQUENCE LISTING (97 sequences)

SEQ ID NO: 1 - protein, 7 aa, Artificial Sequence
  Synthetic
PLGVRGK

SEQ ID NO: 2 - protein, 7 aa, Artificial Sequence
  Synthetic
FPRSGGG

SEQ ID NO: 3 - protein, 10 aa, Artificial Sequence
  Synthetic
GGSGRSANAK

SEQ ID NO: 4 - protein, 5 aa, Artificial Sequence
  Synthetic
GVPRG

SEQ ID NO: 5 - protein, 8 aa, Artificial Sequence
  Synthetic
PVPLSLVM

SEQ ID NO: 6 - protein, 14 aa, Artificial Sequence
  Synthetic
SPSTPPTPSPSTPP

SEQ ID NO: 7 - DNA, 20 bp, Artificial Sequence
  Synthetic
tcatagttagcgtaacgatc

SEQ ID NO: 8 - protein, 13 aa, Artificial Sequence
  Synthetic
KGGPLGVRGKGGC

SEQ ID NO: 9 - RNA, 41 nt, Artificial Sequence
  Synthetic
uaauuucuacuaaguguagaucgucgccguccagcucgacc

SEQ ID NO: 10 - RNA, 41 nt, Artificial Sequence
  Synthetic
uaauuucuacuaaguguagaugaucguuacgcuaacuauga

SEQ ID NO: 11 - RNA, 41 nt, Artificial Sequence
  Synthetic
uaauuucuacuaaguguagauccuggguguuccacagcuga

SEQ ID NO: 12 - DNA, 41 bp, Artificial Sequence
  Synthetic
uaauuucuacuaaguguagauctgtgtttatccgctcacaa

SEQ ID NO: 13 - RNA, 41 nt, Artificial Sequence
  Synthetic
uaauuucuacuaaguguagauugaaguagauauggcagcac

SEQ ID NO: 14 - RNA, 41 nt, Artificial Sequence
  Synthetic
uaauuucuacuaaguguagauacaauaugugcuucuacaca

SEQ ID NO: 15 - DNA, 55 bp, Artificial Sequence
  Synthetic
tagcattccacagacagccctcatagttagcgtaacgatctaaagttttgtcgtc

SEQ ID NO: 16 - DNA, 55 bp, Artificial Sequence; identical to SEQ ID NO: 15
  Synthetic
  misc_feature (1)..(55): Phosphorothioate linkages

SEQ ID NO: 17 - DNA, 55 bp, Artificial Sequence; identical to SEQ ID NO: 15
  Synthetic

SEQ ID NO: 18 - DNA, 55 bp, Artificial Sequence
  Synthetic
gacgacaaaactttagatcgttacgctaactatgagggctgtctgtggaatgcta

SEQ ID NO: 19 - DNA, 20 bp, Artificial Sequence
  Synthetic
  misc_feature (1)..(20): Phosphorothioate linkages
ggtcgagctggacggcgacg

SEQ ID NO: 20 - DNA, 20 bp, Artificial Sequence
  Synthetic
  misc_feature (1)..(20): Phosphorothioate linkages
tcatagttagcgtaacgatc

SEQ ID NO: 21 - DNA, 20 bp, Artificial Sequence
  Synthetic
  misc_feature (1)..(20): Phosphorothioate linkages
tcagctgtggaacacccagg

SEQ ID NO: 22 - DNA, 23 bp, Artificial Sequence
  Synthetic
  misc_feature (1)..(23): Phosphorothioate linkages
gagtaacagacatggaccatcag

SEQ ID NO: 23 - DNA, 20 bp, Artificial Sequence
  Synthetic
  misc_feature (1)..(20): Phosphorothioate linkages
ttgtgagcggataaacacag

SEQ ID NO: 24 - DNA, 20 bp, Artificial Sequence
  Synthetic
  misc_feature (1)..(20): Phosphorothioate linkages
gtgctgccatatctacttca

SEQ ID NO: 25 - DNA, 20 bp, Artificial Sequence
  Synthetic
  misc_feature (1)..(20): Phosphorothioate linkages
tgtgtagaagcacatattgt

SEQ ID NO: 26 - DNA, 22 bp, Artificial Sequence
  Synthetic
  misc_feature (1)..(1): Modified by Cyanine 5 dye
cytcatagttagcgtaacgatc

SEQ ID NO: 27 - DNA, 22 bp, Artificial Sequence
  Synthetic
  misc_feature (1)..(1): Modified by Cyanine 5 dye
  misc_feature (1)..(22): Phosphorothioate linkages
cytcatagttagcgtaacgatc

SEQ ID NO: 28 - DNA, 10 bp, Artificial Sequence
  Synthetic
cgtaacgatc

SEQ ID NO: 29 - DNA, 15 bp, Artificial Sequence
  Synthetic
gttagcgtaacgatc

SEQ ID NO: 30 - DNA, 20 bp, Artificial Sequence
  Synthetic
tcatagttagcgtaacgatc

SEQ ID NO: 31 - DNA, 24 bp, Artificial Sequence
  Synthetic
tcatagttagcgtaacgatctaaa

SEQ ID NO: 32 - DNA, 26 bp, Artificial Sequence
  Synthetic
cagccctcatagttagcgtaacgatc

SEQ ID NO: 33 - DNA, 30 bp, Artificial Sequence
  Synthetic
cagccctcatagttagcgtaacgatctaaa

SEQ ID NO: 34 - DNA, 34 bp, Artificial Sequence
  Synthetic
gacagccctcatagttagcgtaacgatctaaagt

SEQ ID NO: 35 - DNA, 10 bp, Artificial Sequence
  Synthetic
  misc_feature (1)..(10): Phosphorothioate linkages
cgtaacgatc

SEQ ID NO: 36 - DNA, 15 bp, Artificial Sequence
  Synthetic
  misc_feature (1)..(15): Phosphorothioate linkages
gttagcgtaacgatc

SEQ ID NO: 37 - DNA, 20 bp, Artificial Sequence
  Synthetic
  misc_feature (1)..(20): Phosphorothioate linkages
tcatagttagcgtaacgatc

SEQ ID NO: 38 - DNA, 24 bp, Artificial Sequence
  Synthetic
  misc_feature (1)..(24): Phosphorothioate linkages
tcatagttagcgtaacgatctaaa

SEQ ID NO: 39 - DNA, 26 bp, Artificial Sequence
  Synthetic
  misc_feature (1)..(26): Phosphorothioate linkages
cagccctcatagttagcgtaacgatc

SEQ ID NO: 40 - DNA, 30 bp, Artificial Sequence; identical to SEQ ID NO: 33
  Synthetic
  misc_feature (1)..(30): Phosphorothioate linkages

SEQ ID NO: 41 - DNA, 34 bp, Artificial Sequence; identical to SEQ ID NO: 34
  Synthetic
  misc_feature (1)..(34): Phosphorothioate linkages

SEQ ID NO: 42 - DNA, 20 bp, Artificial Sequence
  Synthetic
  misc_feature (1)..(20): Phosphorothioate linkages
  misc_feature (20)..(20): Modified by Dibenzocyclooctyne
ggtcgagctggacggcgacg

SEQ ID NO: 43 - DNA, 20 bp, Artificial Sequence
  Synthetic
  misc_feature (1)..(20): Phosphorothioate linkages
  misc_feature (20)..(20): Modified by Dibenzocyclooctyne
tcatagttagcgtaacgatc

SEQ ID NO: 44 - DNA, 20 bp, Artificial Sequence
  Synthetic
  misc_feature (1)..(20): Phosphorothioate linkages
  misc_feature (20)..(20): Modified by Dibenzocyclooctyne
tcagctgtggaacacccagg

SEQ ID NO: 45 - DNA, 23 bp, Artificial Sequence
  Synthetic
  misc_feature (1)..(23): Phosphorothioate linkages
  misc_feature (23)..(23): Modified by Dibenzocyclooctyne
gagtaacagacatggaccatcag

SEQ ID NO: 46 - DNA, 20 bp, Artificial Sequence
  Synthetic
  misc_feature (1)..(20): Phosphorothioate linkages
  misc_feature (20)..(20): Modified by Dibenzocyclooctyne
ttgtgagcggataaacacag

SEQ ID NO: 47 - DNA, 20 bp, Artificial Sequence
  Synthetic
  misc_feature (1)..(20): Phosphorothioate linkages
  SITE (20)..(20): Modified by Dibenzocyclooctyne
gtgctgccatatctacttca

SEQ ID NO: 48 - DNA, 20 bp, Artificial Sequence
  Synthetic
  misc_feature (1)..(20): Phosphorothioate linkages
  misc_feature (20)..(20): Modified by Dibenzocyclooctyne
tgtgtagaagcacatattgt

SEQ ID NO: 49 - DNA, 20 bp, Artificial Sequence
  Synthetic
  misc_feature (1)..(20): Phosphorothioate linkages
  misc_feature (20)..(20): Modified by Azide
tcatagttagcgtaacgatc

SEQ ID NO: 50 - protein, 13 aa, Artificial Sequence
  Synthetic
  SITE (2)..(3): Modifed by azide side chain and photocleavable linker
KGGPLGVRGKGGC

SEQ ID NO: 51 - protein, 10 aa, Artificial Sequence
  Synthetic
  SITE (2)..(3): Modifed by azide side chain and photocleavable linker
KGGFPRSGGC

SEQ ID NO: 52 - protein, 15 aa, Artificial Sequence
  Synthetic
  SITE (2)..(3): Modifed by azide side chain and photocleavable linker
KGGGSGRSANAKGGC

SEQ ID NO: 53 - protein, 9 aa, Artificial Sequence
  Synthetic
  SITE (2)..(3): Modifed by azide side chain and photocleavable linker
KGGVPRGGC

SEQ ID NO: 54 - protein, 13 aa, Artificial Sequence
  Synthetic
  SITE (2)..(3): Modifed by azide side chain and photocleavable linker
KGPVPLSLVMGGC

SEQ ID NO: 55 - protein, 12 aa, Artificial Sequence
  Synthetic
  SITE (1)..(1): Modifed by N-terminal Fluorescein fluorophore
  SITE (10)..(11): Modifed by CPQ2 and PEG2
GGPQGIWGQKGC

SEQ ID NO: 56 - protein, 12 aa, Artificial Sequence
  Synthetic
  SITE (1)..(1): Modifed by N-terminal Fluorescein fluorophore
  SITE (10)..(11): Modifed by CPQ2 and PEG2
GGLVPRGSGKGC

SEQ ID NO: 57 - protein, 11 aa, Artificial Sequence
  Synthetic
  SITE (1)..(1): Modifed by N-terminal Fluorescein fluorophore
  SITE (9)..(10): Modifed by CPQ2 and PEG2
GGPVGLIGKGC

SEQ ID NO: 58 - protein, 13 aa, Artificial Sequence
  Synthetic
  SITE (1)..(1): Modifed by N-terminal Fluorescein fluorophore
  SITE (11)..(12): Modifed by CPQ2 and PEG2
GGPWGIWGQGKGC

SEQ ID NO: 59 - protein, 13 aa, Artificial Sequence
  Synthetic
  SITE (1)..(1): Modifed by N-terminal Fluorescein fluorophore
  SITE (11)..(12): Modifed by CPQ2 and PEG2
GGPVPLSLVMKGC

SEQ ID NO: 60 - protein, 12 aa, Artificial Sequence
  Synthetic
  SITE (1)..(1): Modifed by N-terminal Fluorescein fluorophore
  SITE (10)..(11): Modifed by CPQ2 and PEG2
GGPLGLRSWKGC

SEQ ID NO: 61 - protein, 12 aa, Artificial Sequence
  Synthetic
  SITE (1)..(1): Modifed by N-terminal Fluorescein fluorophore
  SITE (10)..(11): Modifed by CPQ2 and PEG2
GGPLGVRGKKGC

SEQ ID NO: 62 - protein, 11 aa, Artificial Sequence
  Synthetic
  SITE (1)..(1): Modifed by N-terminal Fluorescein fluorophore
  SITE (3)..(4): Modifed by pipecolic acid
  SITE (9)..(10): Modifed by CPQ2 and PEG2
GGFRSGGGKGC

SEQ ID NO: 63 - protein, 11 aa, Artificial Sequence
  Synthetic
  SITE (1)..(1): Modifed by N-terminal Fluorescein fluorophore
  SITE (9)..(10): Modifed by CPQ2 and PEG2
GGFPRSGGGGC

SEQ ID NO: 64 - protein, 11 aa, Artificial Sequence
  Synthetic
  SITE (1)..(1): Modifed by N-terminal Fluorescein fluorophore
  SITE (3)..(4): Modifed by pipecolic acid
  SITE (9)..(10): Modifed by CPQ2 and PEG2
GGFKSGGGKGC

SEQ ID NO: 65 - protein, 15 aa, Artificial Sequence
  Synthetic
  SITE (1)..(1): Modifed by N-terminal Fluorescein fluorophore
  SITE (13)..(14): Modifed by CPQ2 and PEG2
GGGSGRSANAKGKGC

SEQ ID NO: 66 - protein, 13 aa, Artificial Sequence
  Synthetic
  SITE (1)..(1): Modifed by N-terminal Fluorescein fluorophore
  SITE (11)..(12): Modifed by CPQ2 and PEG2
GILSRIVGGGKGC

SEQ ID NO: 67 - protein, 10 aa, Artificial Sequence
  Synthetic
  SITE (1)..(1): Modifed by N-terminal Fluorescein fluorophore
  SITE (8)..(9): Modifed by CPQ2 and PEG2
GGVPRGGKGC

SEQ ID NO: 68 - protein, 13 aa, Artificial Sequence
  Synthetic
  SITE (1)..(1): Modifed by N-terminal Fluorescein fluorophore
  SITE (11)..(12): Modifed by CPQ2 and PEG2
GSGSKIIGGGKGC

SEQ ID NO: 69 - protein, 13 aa, Artificial Sequence
  Synthetic
  SITE (1)..(1): Modifed by N-terminal Fluorescein fluorophore
  SITE (11)..(12): Modifed by CPQ2 and PEG2
GPVPLSLVMGKGC

SEQ ID NO: 70 - protein, 14 aa, Artificial Sequence
  Synthetic
  SITE (1)..(1): Modifed by N-terminal Fluorescein fluorophore
  SITE (11)..(12): Modifed by CPQ2
  SITE (13)..(14): Modifed by PEG2
GGLGPKGQTGKKKC

SEQ ID NO: 71 - protein, 162 aa, Artificial Sequence
  Synthetic
MEVQLVESGGGLVQPGGSLRLSCAASGFILDYYAIGWFRQAPGKEREGVLCIDASDDITYYADSVKGRFTISRDNAKNTVYLQMNSLKPEDTGVYYCATPIGLSSSCLLEYDYDYWGQGTLVTVSSGSHHHHHHSPSTPPTPSPSTPPGSGRSANAKGGGSC

SEQ ID NO: 72 - protein, 164 aa, Artificial Sequence
  Synthetic
MAQVQLVESGGRLVQAGDSLRLSCAASGRTFSTSAMAWFRQAPGREREFVAAITWTVGNTILGDSVKGRFTISRDRAKNTVDLQMDNLEPEDTAVYYCSARSRGYVLSVLRSVDSYDYWGQGTQVTVSGSHHHHHHSPSTPPTPSPSTPPGSGRSANAKGGGSC

SEQ ID NO: 73 - protein, 1228 aa, Artificial Sequence
  Synthetic
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 74 - RNA, 20 nt, Artificial Sequence
  Synthetic
gaucguuacgcuaacuauga

SEQ ID NO: 75 - protein, 7 aa, Artificial Sequence
  Synthetic
PQGIWGQ

SEQ ID NO: 76 - protein, 7 aa, Artificial Sequence
  Synthetic
LVPRGSG

SEQ ID NO: 77 - protein, 6 aa, Artificial Sequence
  Synthetic
PVGLIG

SEQ ID NO: 78 - protein, 8 aa, Artificial Sequence
  Synthetic
PWGIWGQG

SEQ ID NO: 79 - protein, 7 aa, Artificial Sequence
  Synthetic
PLGVRFK

SEQ ID NO: 80 - protein, 6 aa, Artificial Sequence
  Synthetic
  SITE (1)..(2): Linked by pipecolic acid
FRSGGG

SEQ ID NO: 81 - protein, 6 aa, Artificial Sequence
  Synthetic
  SITE (1)..(2): Linked by pipecolic acid
FKSGGG

SEQ ID NO: 82 - protein, 8 aa, Artificial Sequence
  Synthetic
ILSRIVGG

SEQ ID NO: 83 - protein, 8 aa, Artificial Sequence
  Synthetic
SGSKIIGG

SEQ ID NO: 84 - protein, 9 aa, Artificial Sequence
  Synthetic
GLGPKGQTG

SEQ ID NO: 85 - RNA, 41 nt, Artificial Sequence; identical to SEQ ID NO: 10
  Synthetic
  misc_feature (22)..(41): Phosphorothioate linkages

SEQ ID NO: 86 - RNA, 41 nt, Artificial Sequence; identical to SEQ ID NO: 10
  Synthetic
  misc_feature (31)..(41): Phosphorothioate linkages

SEQ ID NO: 87 - RNA, 41 nt, Artificial Sequence; identical to SEQ ID NO: 10
  Synthetic
  misc_feature (37)..(41): Phosphorothioate linkages

SEQ ID NO: 88 - RNA, 41 nt, Artificial Sequence; identical to SEQ ID NO: 10
  Synthetic
  modified_base (22)..(41): 2′-O-Methoxyethyl nucleotide

SEQ ID NO: 89 - RNA, 41 nt, Artificial Sequence; identical to SEQ ID NO: 10
  Synthetic
  modified_base (32)..(41): 2′-O-Methoxyethyl nucleotide

SEQ ID NO: 90 - RNA, 41 nt, Artificial Sequence; identical to SEQ ID NO: 10
  Synthetic
  modified_base (38)..(41): 2′-O-Methoxyethyl nucleotide

SEQ ID NO: 91 - RNA, 20 nt, Artificial Sequence
  Synthetic
  misc_feature (1)..(6): Phosphorothioate linkages
  modified_base (7)..(16): 2′-O-Methoxyethyl nucleotide
  misc_feature (16)..(20): Phosphorothioate linkages
ggucgagcuggacggcgacg

SEQ ID NO: 92 - RNA, 20 nt, Artificial Sequence
  Synthetic
  misc_feature (1)..(6): Phosphorothioate linkages
  modified_base (7)..(16): 2′-O-Methoxyethyl nucleotide
  misc_feature (16)..(20): Phosphorothioate linkages
ucauaguuagcguaacgauc

SEQ ID NO: 93 - RNA, 20 nt, Artificial Sequence
  Synthetic
  misc_feature (1)..(6): Phosphorothioate linkages
  modified_base (7)..(16): 2′-O-Methoxyethyl nucleotide
  misc_feature (16)..(20): Phosphorothioate linkages
ucagcuguggaacacccagg

SEQ ID NO: 94 - RNA, 23 nt, Artificial Sequence
  Synthetic
  misc_feature (1)..(6): Phosphorothioate linkages
  modified_base (7)..(19): 2′-O-Methoxyethyl nucleotide
  misc_feature (16)..(17): Phosphorothioate linkages
  misc_feature (19)..(23): Phosphorothioate linkages
gaguaacagacauggaccaucag

SEQ ID NO: 95 - RNA, 20 nt, Artificial Sequence
  Synthetic
  misc_feature (1)..(6): Phosphorothioate linkages
  modified_base (7)..(16): 2′-O-Methoxyethyl nucleotide
  misc_feature (16)..(20): Phosphorothioate linkages
uugugagcggauaaacacag

SEQ ID NO: 96 - RNA, 20 nt, Artificial Sequence
  Synthetic
  misc_feature (1)..(6): Phosphorothioate linkages
  modified_base (7)..(16): 2′-O-Methoxyethyl nucleotide
  misc_feature (16)..(20): Phosphorothioate linkages
gugcugccauaucuacuuca

SEQ ID NO: 97 - RNA, 20 nt, Artificial Sequence
  Synthetic
  misc_feature (1)..(6): Phosphorothioate linkages
  modified_base (7)..(16): 2′-O-Methoxyethyl nucleotide
  misc_feature (16)..(20): Phosphorothioate linkages
uguguagaagcacauauugu

(19) United States
(12) Patent Application Publication
(10) Pub. No.: US 2021/0262025 A1
(43) Pub. Date: Aug. 26, 2021

(54) MULTIPLEXED IN VIVO DISEASE SENSING WITH NUCLEIC ACID-BARCODED REPORTERS

(71) Applicant: Massachusetts Institute of Technology, Cambridge, MA (US)

(72) Inventors: Sangeeta N. Bhatia, Lexington, MA (US); Liangliang Hao, Cambridge, MA (US); Renee Zhao, Los Angeles, CA (US); Chayanon Ngambenjawong, Cambridge, MA (US)

(73) Assignee: Massachusetts Institute of Technology, Cambridge, MA (US)

(21) Appl. No.: 17/124,999

(22) Filed: Dec. 17, 2020

Related U.S. Application Data
(60) Provisional application No. 62/977,817, filed on Feb. 18, 2020.

(51) Int. Cl. C12Q 1/6874; C12N 9/22

(52) U.S. Cl. CPC C12Q 1/6874; C12N 2310/20; C12N 9/22

(57) ABSTRACT

Aspects of the present disclosure relate to methods and compositions useful for in vivo and/or in vitro profiling of environmental triggers (e.g., enzyme activity, pH or temperature). In some embodiments, the disclosure provides methods of in vivo enzymatic processing of exogenous molecules followed by detection of modified nucleic acid barcodes as representative of the presence of active enzymes (e.g., proteases) associated with a disease, for example, cancer. In some embodiments, the disclosure provides compositions and methods for production of in vivo sensors comprising modified nucleic acid barcodes.

### RELATED APPLICATIONS

This application claims priority under 35 U.S.C. § 119(e) to U.S. Provisional Application No. 62/977,817, filed Feb. 18, 2020 and entitled “MULTIPLEXED IN VIVO DISEASE SENSING WITH NUCLEIC ACID-BARCODED REPORTERS,” which is incorporated herein by reference in its entirety for all purposes.

### GOVERNMENT SUPPORT

This invention was made with Government support under Grant No. CA237861 awarded by the National Institutes of Health (NIH). The Government has certain rights in the invention.

### BACKGROUND

The interplay between the cellular microenvironment and malignant cells is often a key determinant of disease progression. For example, characteristics of the tumor microenvironment including extracellular matrix (ECM) alterations, pH, stromal composition, or immune components have been found to be important factors in driving metastatic dissemination across cancers. As tumors start to invade, they often alter the ECM architecture through aberrant proteolytic activities. Dysregulation of proteases in cancer has important consequences in cell signaling and helps drive cancer cell proliferation, invasion, angiogenesis, avoidance of apoptosis, and metastasis. To promote precision medicine, efficient and noninvasive methods of characterizing protein activity and cellular microenvironments are needed.

### SUMMARY

Aspects of the present disclosure provide a sensor comprising a scaffold linked to a modified nucleic acid barcode that is capable of being released from the sensor when exposed to an environmental trigger in vivo. In some embodiments, the environmental trigger is an enzyme present in a subject.

In some embodiments, the modified nucleic acid barcode comprises a modified internucleoside linkage, a modified nucleotide, and/or a terminal modification.

In some embodiments, the modified internucleoside linkage is selected from a phosphorothioate linkage or a boranophosphate linkage.

In some embodiments, the modified nucleic acid barcode comprises at least two different modifications.

In some embodiments, the modified nucleic acid barcode comprises a modified sugar moiety and/or a modified base. In some embodiments, the modified sugar moiety comprises a 2′-OH group modification and/or a bridging moiety. In some embodiments, the 2′-OH group modification is selected from the group consisting of 2′-O-Methyl (2′-O-Me), 2′-Fluoro (2′-F), and 2′-O-methoxy-ethyl (2′-O-MOE). In some embodiments, the modified base is a deoxyuridine (dU), a 5-Methyl deoxyCytidine (5-methyl dC), or an inverted dT. In some embodiments, the bridging moiety is a locked nucleic acid.

In some embodiments, the terminal modification is a 5′ terminal modification phosphate modification, a 5′-phosphorylation, or a 3′-phosphorylation.

In some embodiments, each internucleotide linkage is a phoshporothioate linkage.

In some embodiments, the modified nucleic acid barcode is single-stranded or double-stranded.

In some embodiments, the nucleic acid barcode is 20 nucleotides in length.

In some embodiments, the modified nucleic acid barcode comprises a deoxyribonucleotide and/or a ribonucleotide.

In some embodiments, the modified nucleic acid barcode is capable of activating the single-stranded nucleic acid cleavage activity of a Cas protein in the presence of a CRISPR RNA sequence (crRNA).

In some embodiments, the Cas protein is a type V Cas protein, a type VI Cas protein, a Cas14, a CasX, a CasZ, or a CasY, optionally wherein the type VI Cas protein is Cas 13a or Cas 13b.

In some embodiments, the scaffold is an antibody.

In some instances, the modified nucleic acid barcode comprises a sequence that is at least 80% identical to SEQ ID NOs: 16, 19-27, or 35-49 or a sequence from Table 11.

In some instances, the modified nucleic acid is linked to an enzyme-cleavable substrate that is linked to the scaffold.

In some instances, the enzyme-cleavable substrate comprises a sequence that is at least 80% identical to a sequence selected from SEQ ID NOs: 50-70. Further aspects of the present disclosure provide a method of detecting an enzyme that is active in a subject comprising: obtaining a sample from a subject who has been administered any of the sensors described herein; and detecting the modified nucleic acid barcode, wherein detection of the modified nucleic acid is indicative of the enzyme being in the active form in the subject.

In some embodiments, detecting the modified nucleic acid barcode comprises contacting the sample with a system that comprises: (i) a crRNA sequence that comprises a guide sequence that is complementary to a sequence in the modified nucleic acid barcode; (ii) a Cas protein; and (iii) a reporter that comprises a first ligand that is connected to a second ligand through a single-stranded nucleic acid linker, wherein the single-stranded nucleic acid linker is not complementary to the guide sequence; and detecting cleavage of the reporter.

In some embodiments, the reporter is a fluorescently quenched reporter and detecting cleavage of the reporter comprises detecting an increase in fluorescence as compared to the level of fluorescence detected in the system in the absence of the sample from the subject; or the first ligand binds a different antibody as compared to the second ligand and detecting cleavage of the reporter comprises using a lateral flow assay.

In some instances, the crRNA sequence comprises a sequence that is at least 80% identical to a sequence selected from SEQ ID NOs: 9-14 or Table 10.

### BRIEF DESCRIPTION OF DRAWINGS

FIG. 1 shows a non-limiting example of nucleic acid-barcoded sensors (e.g., DNA-barcoded sensors) for detection and imaging of cancer metastasis. Nucleic acid-barcoded sensors are comprised of a nano-carrier (synthetic or biologic) functionalized with proteolytic-activated short peptides barcoded with oligonucleotides (i). After in vivo administration, activation of nucleic acid-barcoded sensors by disease-specific protease activity triggers release of synthetic nucleic acid barcodes (ii) that are size-specifically concentrated in the urine for sensitive detection (iii). Nucleic acid barcodes in the urine activate programmable CRISPR enzymes to release the multiplexed reporter signals that may be fluorescent or detected on paper (iv), allowing for in situ classification at the point-of-care via the patterns of local proteolytic activities in the disease microenvironment (v).

FIGS. 2A-2G show chemically modified DNA enables CRISPR-based urinary readout for in vivo sensing. FIG. 2A shows DNA fragments activate nonspecific ssDNase cleavage upon binding to crRNA on Cas12a. Such activity can be tracked by the release of the quenched fluorescent reporter. For example, the quenched fluorescent reporter may be 5′-FAM-T(10)-3′IABkFQ. FIG. 2B depicts cleavage of the fluorescent reporter by Cas12a activated by native dsDNA, ssDNA, and chemically modified ssDNA. A representative Michaelis-Menten plot of LbaCAS12a-catalyzed ssDNA trans cleavage using native dsDNA, ssDNA, or fully phosphorothioate-modified ssDNA activator is shown. The initial reaction velocity (V₀) is determined from the slope of the curve at the beginning of the reaction. FIG. 2C shows schematic showing urine testing in a mouse model and study time course (1 h). FIG. 2D depicts trans-cleavage rate of native or modified ssDNA collected in the mouse urine. The effect of the length of the ssDNA activator on the reaction rate for in vitro and in vivo applications were assessed by quantifying the trans-cleavage rate of Cas12a upon activation of native or modified ssDNA in solution (4 nM) or mouse urine (1 nmol per injection). The trans-cleavage rates in each condition in the format of initial reaction velocity were normalized to that of a 24-mer (See also Table 6). FIG. 2E shows heatmap of trans-cleavage rate of different crRNA-modified ssDNA activator pairs. Assays were performed with urine samples collected from mice injected with 1 nmol of modified ssDNA activator after 1 h of i.v. administration. FIG. 2F is a schematic showing set up of paper-based lateral flow assay (left). Different bands were visible on paper strip once the Cas12a was activated by mouse urine with and without DNA activator (as shown on the right). Band intensities were quantified using ImageJ and each curve was aligned below the corresponding paper strip. In the curve indicating the position and intensity of the bands on the paper strip, the top peak shows the presence of the “sample band” and the bottom peak shows the presence of the “control band”. The presence of the “sample band” indicates that the cleaved reporters exist, showing the Cas12a was activated by the DNA activator. For example, when Cas12a is activated by the DNA activator in mouse urine, it may cleave the fluorescein (FAM)-biotin-paired oligonucleotide reporter and free the FAM molecule that may be detected on the “′sample band.” Uncleaved reporters are trapped on the “control band” via binding of biotin to streptavidin. Different bands are visible on paper strips (right). Band intensities were quantified using ImageJ, and each curve was aligned below the corresponding paper strip. The top peak of the curve shows the freed FAM molecule in cleaved reporter samples, and the bottom peak shows the presence of the uncleaved FAM-biotin reporter. FIG. 2G shows Michaelis-Menten plot of LbaCas12a-catalyzed ssDNA trans-cleavage upon a representative DNA-crRNA pairing (complementary sequences are shown) on paper. Data were plotted with the quantified band intensity of cleaved reporter on paper strips. The top sequence is SEQ ID NO: 30 and the bottom sequence is SEQ ID NO: 74.

FIGS. 3A-3F show disease-associated proteases for urinary DNA barcode release. FIG. 3A is a schematic showing design of DNA-barcoded protease-activated nanosensors. DNA-barcoded protease-activated peptide is immobilized on a nano-carrier for size-specific release of the barcode in urine. SEQ ID NO: 7 is used as a non-limiting example of a barcode and SEQ ID NO: 8 is used as a non-limiting example of a protease-sensitive peptide. The chemical structure between SEQ ID NO: 7 and SEQ ID NO: 8 is an internal UV-sensitive residue (3-amino-3-(2-nitrophenyl)propionic acid) that allows for the recovery of DNA barcode by photolysis from urinary cleavage fragments after in vivo proteolysis. FIG. 3B shows TCGA data analysis of fold change of well-studied metallo- or serine-protease mRNA expression in tumors compared to healthy controls. FIG. 3C shows proteases in FIG. 3B identified in the matrix of primary human colon cancer (PC), or liver metastases (LM), in comparison with normal colon (COL) and liver (L) tissues. FIG. 3D shows ROC curves constructed based on protease mRNA expression data in FIG. 3B to represent how well protease dysregulation can classify various cancer types compared to healthy controls. FIG. 3E shows FRET-paired protease substrates, consisting of a peptide sequence flanked by a FAM fluorophore and a CPQ-2 quencher, were screened against recombinant matrix metalloproteinases or tissue lysates from tumor-bearing or control mice. FIG. 3F depicts a heatmap with fluorescence fold changes after cleavage was monitored with kinetic plate reader. In FIG. 3F, f represents phenylalanine as d-amino acid and Pip represents pipecolic acid. SEQ ID NOs for sequences shown in FIG. 3F are as follows: PQGIWGQ (SEQ ID NO: 75); LVPRGSG (SEQ ID NO: 76); PVGLIG (SEQ ID NO: 77); PWGIWGQG (SEQ ID NO: 78); PVPLSLVM (SEQ ID NO: 5); PLGVRFK (SEQ ID NO: 79); f-Pip-RSGGG (SEQ ID NO: 80); fPRSGGG (SEQ ID NO: 2); f-Pip-KSGGG (SEQ ID NO: 81); GGSGRSANAK (SEQ ID NO: 3); ILSRIVGG (SEQ ID NO: 82); GVPRG (SEQ ID NO: 4); SGSKIIGG (SEQ ID NO: 83); PVPLSLVM (SEQ ID NO: 5); GLGPKGQTG (SEQ ID NO: 84).

FIGS. 4A-4D depict multiplexed DNA-barcoded activity-based nanosensors (ABNs) for longitudinal disease monitoring. FIG. 4A depicts a non-limiting workflow that was used for longitudinal disease detection and monitoring with the multiplexed DNA-barcoded ABNs platform. FIG. 4B shows histological staining of lung sections of Balb/c mice bearing CRC lung nodules (left) and immunohistochemistry of the same tissue stained with anti-PEG (middle) or epitope control antibody (right). FIG. 4C shows pooled DNA-barcoded ABNs were administered to tumor-bearing and control animals at day 11 or 21 after tumor initiation, bladder was voided and urine was collected at 1 hr. Cas12a trans-cleavage assay with fluorescent-reporter was performed against each DNA-barcode, initial cleavage rate was calculated and plotted. FIG. 4D shows paper-based LFA of Cas12a activated by mouse urine samples collected in FIG. 4C with quantification of bands intensities by ImageJ. In each graph shown in FIG. 4D, the intensity of control bands and sample bands on paper strips with urine samples from sham mice and tumor-bearing mice were quantified and curves indicating the position and intensity of the bands on the paper strip were aligned below each paper strip. The top peak shows the presence of the “sample band” and the bottom peak shows the presence of the “control band”.

FIGS. 5A-5H show localization and activity experiments involving tumor-targeted DNA-barcoded ABNs. FIG. 5A shows nanobody (VHH domain) derived from camelid IgG on the left and generation of a protease-activatable nanobodies with an unpaired cysteine and 1-step conjugation of DNA-barcoded urinary reporter on the right. FIG. 5B is a schematic showing urine testing in a human prostate cancer xenograft model and generation of the DNA-encoded protease-activatable nanobody with an unpaired cysteine and 1-step conjugation of ssDNA activator (i). Activation of diagnostic by disease-specific protease activity triggers (ii) release of ssDNA activator into urine for disease detection (iii). Study time course of urine testing and detection of the ssDNA activator with Cas12a trans-cleavage assay using fluorescent or paper readout (iv). t, time at given testing step; tᵢ, time of sensor injection; tᵤ, time of urine collection. FIG. 5C is an IVIS image that shows biodistribution of cMET targeting nanobody and on-targeting GFP nanobody when injected intravenously in nude mice bearing PC-3 xenografts. Scale bar=1 cm. FIG. 5D shows unprocessed urine samples collected from tumor-bearing mice injected with DNA-encoded cMET nanobody or DNA-encoded GFP nanobody, and healthy control mice injected with DNA-encoded cMET nanobody were applied in the Cas12a trans-cleavage assay. Initial reaction velocity (V₀) of the Cas12a trans-cleavage assays were calculated and normalized to that of healthy control mice (n=5 or 7 mice per group; ±SEM; unpaired t-test with Welch's correction, *P<0.05).

FIG. 5E shows the same results as FIG. 5D in which urine samples collected from tumor-bearing and healthy control mice were applied in a LbaCas12a trans-cleavage assay. LbaCas12a activated by urine collected from tumor-bearing mice injected with DNA conjugation of a non-targeting nanobody against green fluorescent protein (GFP) served as negative control. FIG. 5F shows immunofluorescent staining of Cy7-labeled DNA-encoded cMET nanobody and DNA-encoded, non-targeting GFP nanobody on sections of PC-3 tumors. Scale bar=20 μm. FIG. 5G shows the results of a paper-based LFA of LbaCas12a activated by urine samples collected from tumor-bearing or healthy control mice in FIG. 5D. Band intensities were quantified using ImageJ and each curve was aligned below the corresponding paper strip. The top peak of the curve shows the presence of the cleaved reporter and the bottom peak shows the presence of the uncleaved reporter. FIG. 5H shows ROC curves characterize the predictive power of a biomarker by returning the area under the curve (AUC) as a metric, with a baseline AUC of 0.5 representing a random biomarker classifier. AUC comparison between DNA-encoded cMET nanobody or DNA-encoded GFP nanobody injected tumor cohort against normal cohort in FIGS. 5D and 5G. Dashed line represents an AUC of 0.5, and a perfect AUC is 1.0.

FIGS. 6A-6E show experiments relating to portable monitoring invasive CRC using DNA-encoded multiplex synthetic urine biomarkers. FIG. 6A shows a scheme of the work flow for longitudinal disease monitoring with the multiplexed DNA-encoded synthetic urine biomarkers. FIG. 6B shows a diagram depicting a Forster resonance energy transfer (FRET)-based peptide assay to identify the real-time cleavage of peptide substrates by invasive CRC tissue homogenates collected 21 days after tumor inoculation. Peptide cleavage kinetics were monitored and cleavage rates were plotted (n=5 mice per group; ±SEM; unpaired t-test with Welch's correction, **P<0.01, ****P<0.0001). FIG. 6C shows graphs of a Cas12a trans-cleavage assay performed against various DNA-barcodes after pooled DNA-SUBs were administered to Balb/c mice bearing CRC lung tumor nodules (tumor) and saline-injected control animals (sham) at day 11 or 21 after tumor initiation. All urine samples were collected at 1 h after sensor administration. Cas12a trans-cleavage assays were performed against each DNA-barcode with the fluorophore-quencher paired reporter. Initial reaction velocity (V₀) of the Cas12a trans-cleavage assays were calculated and normalized to that of saline injected control animals (n=8 or 10 mice per group; ±SEM; unpaired t-test with Welch's correction, *P<0.05, **P<0.01). The initial reaction velocity (V₀) refers to the slope of the curve at the beginning of a reaction. FIG. 6D shows images of representative paper strips of the paper-based LFA of Cas12a activated by mouse urine samples collected in FIG. 6C. Band intensities were quantified using ImageJ. The top peak of the curve shows the freed FAM molecule in cleaved reporter and the bottom peak shows the presence of the uncleaved FAM-biotin reporter. FIG. 6E left graph shows an ROC curve analysis indicates predictive ability of single or combined DNA-SUBs with fluorescent readout in FIG. 6D. FIG. 6E right graph shows an ROC curve shows the predictive ability of paper-based urinary readout in FIG. 6D. ROC analysis utilized ratio of quantified cleaved reporter band intensity over its corresponding control band intensity. Dashed line represents an AUC of 0.5, and a perfect AUC is 1.0.

FIGS. 7A-7H show collateral activity of LbaCas12a activated by different types of DNA activators. FIG. 7A shows the urine signal after systemic administration of modified and native 20-mer DNAs showing amplification kinetics of modified DNA that surpassed the steady-state concentration of its native DNA counterpart. Signal maximized at 1 hour after administration of DNAs. Image shows urine samples on 384-well plate visualized on the LI-COR Odyssey CLx system. Urine fluorescence was normalized to that of the first timepoint of Cy5-modified DNA injected animal (30 min after DNA injection; n=3 per condition). FIGS. 7B-7H shows trans-cleavage rates of Cas12a upon activation of different modified ssDNA activator-crRNA pairs were determined in the Cas12a fluorescent cleavage assay. Assays were performed with urine samples collected from mice injected with 1 nmol of modified ssDNA activator after 1 h of i.v. administration. The initial reaction velocity (V₀) is determined from the slope of the curve at the beginning of a reaction.

FIGS. 8A-8H show characterization of dose dependence of LbaCas12a activation by DNA activators using fluorescent readout. FIGS. 8A-8G show LbaCas12a catalyzed ssDNA trans-cleavage using phosphorothioate-modified 20-mer ssDNA activators. Trans-cleavage rates of Cas12a upon activation of different modified ssDNA activator-crRNA pairs were determined in the Cas12a fluorescent cleavage assay. Assays were performed with different concentration of modified ssDNA activator (8 nM, 4 nM, 2 nM, 1 nM, 0.5 nM, 0.25 nM, 0.125 nM or 0 nM), with increasing slope to the intensity over time curve observed with increasing concentration of activator. FIG. 8H shows the initial reaction velocity (V₀) is determined from the slope of the curve at the beginning of a reaction in FIGS. 8A-8G and plotted to determine the linear range of assay performance. Linear regions were shown in V₀ of reactions for all modified ssDNA activator-crRNA pairs within 1 nM of DNA activators. DNA activator 1, 2, 3, 5, 6 were selected for construction of in vivo sensors because of their similarity in assay performance. Sequences of oligonucleotides were shown in Table 5.

FIGS. 9A-9G show characterization of dose dependence of LbaCas12a activation by DNA activators using lateral flow assay. LbaCas12a catalyzed ssDNA trans cleavage using phosphorothioate-modified 20-mer ssDNA activators. Trans-cleavage rates of Cas12a upon activation of different modified ssDNA activator-crRNA pairs were determined in the Cas12a lateral flow assay. Assays were performed with different concentration of modified ssDNA activator (8 nM, 4 nM, 2 nM, 1 nM, 0.5 nM, 0.25 nM, 0.1 nM or 0 nM) and dual labeled FAM-T10-Biotin reporter. Resulting solution was mixed with HybriDetect 1 assay buffer. HybriDetect 1 lateral flow strips were dipped into solution and intensity of cleaved reporter bands was quantified in ImageJ and plotted to fit Michaelis-Menten kinetics. Consistent with the Cas12a fluorescent cleavage assay, linear regions were found within 1 nM of DNA activators for all modified ssDNA activator-crRNA pairs tested. Sequences of oligonucleotides were shown in Table 5.

FIGS. 10A-10E show characterization of DNA-conjugated nanobody in vitro and in vivo. FIG. 10A shows separation of DNA-conjugated cMET nanobody in a size exclusion chromatography. UV 260 nm (red), the elution curve of oligonucleotides; UV 280 nm (red), the elution curve of proteins. Red shed indicates the elution of the DNA-nanobody conjugate that has significant absorbance at both 260 nm and 280 nm. FIG. 10B shows SDS-PAGE analysis of the DNA-nanobody conjugate showing predicted molecular weight. FIG. 10C shows increased expression of cMET, the biomarker that the nanobody targets and PLAU, the protease triggers the DNA barcode release, in prostate cancer line PC-3 compared with normal prostate epithelial line RWPE1. FIG. 10D shows immunohistochemical staining of cMET and PLAU in PC-3 flank tumors. Brown, positive staining. Blue, nuclei. Scale bar=200 μm. FIG. 10E shows caliper quantification of tumor sizes of animals shown in FIGS. 3D-3E. Tumor-bearing mice were injected with different types of DNA-conjugated nanobodies.

FIGS. 11A-11D show characterization of DNA-encoded synthetic urine biomarker built on the polymeric PEG core. FIG. 11A shows characterization of the representative DNA-PAP7-SUB on a PEG core. HPLC purification of peptide-DNA (PAP7-DNA2) conjugate. The conjugate was analyzed in mass-spectrometry and showed expected molecular weight (8283 Da). FIG. 11B shows FPLC purification of sensor showed separation of functionalized sensor and unbounded peptide-DNA conjugate. FIG. 11C shows dynamic light scattering analysis showed increase of particle size from 8.3 nm (PEG core only) and 13 nm (functionalized sensor). FIG. 11D shows plasma half-life shows rapid clearance of native DNA molecules and prolonged half-life of the modified DNA and PEG scaffold in healthy Balb/c mice.

FIGS. 12A-12E show histology of major organs of CRC lung metastasis model. Immunocompetent Balb/c mice were injected with MC26-Fluc cells (tumor) or saline (sham) intravenously. FIGS. 12A-12E show organs (lung, liver, kidney, heart and spleen) were collected at 11 and 21 days after administration. Organs were fixed, embedded in paraffin, and stained with hematoxylin & eosin. Study was done with n=3 mice per time point and images from a representative animal are shown. Scale bar=100 μm. Arrows, tumor nodules in the lung.

FIGS. 13A-13D show identification of deregulated proteases in CRC to select peptide substrates for in vivo sensors. FIG. 13A shows RT-qPCR validation of proteases in the tumor-bearing lung from Balb/c mice injected with MC26-Fluc cells in comparison of normal lung from Balb/c mice injected with saline. FIG. 13B shows typical proteases identified in the matrix of primary human colon cancer (CC) and their liver metastases (LM), in comparison of normal colon (Nor.) tissue. Pink, presence; white, absence. Data available from Matrisome project (http://matrisomeproject.mit.edu/). FIG. 13C shows immunofluorescence staining of proteases in the tumor bearing lung tissue sections. Staining of MMP3, MMP7, MMP9 and CTSD is shown in red. Nuclei are counterstained blue with DAPI. Scale bar=100 μm. FIG. 13D shows 16 FRET-paired protease substrates, each consisting of a peptide sequence flanked by a FAM fluorophore and a CPQ-2 quencher, were screened against 22 recombinant proteolytic enzymes. Lower, FRET signal was monitored by kinetic plate reader and the z-scored cleavage rate were subjected to heatmap and Hierarchical Clustering on Morpheus (software.broadinstitute.org/morpheus). Asterisk, peptide substrates selected to build in vivo sensors because of their broad coverage of metallo, serine and aspartic protease activities.

FIG. 14 shows selected FRET-paired protease substrates (PAP 7, PAP 9, PAP 11, PAP 13, PAP 15) were incubated against tissue lysates from tumor bearing lung (tumor, upper) or normal lung (sham, lower) of Balb/c mice (n=5).

FIGS. 15A-15C show multiplexed DNA-encoded synthetic urine biomarkers for disease monitoring. FIG. 15A shows a schematic of the work flow for longitudinal disease monitoring with the multiplexed DNA-encoded synthetic urine biomarkers. FIG. 15B shows 5-plex DNA-SUBs were pooled and administered to Balb/c mice bearing CRC lung tumor nodules and control animals at day 11 or 21 after tumor initiation. All urine samples were collected at 1 h after sensor administration. Two sensors (DNA-PAP11-SUB, DNA-PAP13-SUB) showed an increase in the sets of tumor-bearing mice generated urine signals that were elevated relative to control animals. FIG. 15C shows representative paper strips of the paper-based LFA of Cas12a activated by mouse urine samples collected in FIG. 15B. Band intensities were quantified using ImageJ. The top peak of the curve shows the presence of the “cleaved reporter” and the bottom peak shows the presence of the “control band.”

### DETAILED DESCRIPTION

While genetic alterations underlie many diseases including cancer, mutation data often provides no insight into protein activity or the presence of other environmental triggers at sites of disease including pH. Similarly, protein levels are not always correlated with activity. Since aberrant protein activity and changes in the tissue microenvironment are often the ultimate downstream effectors of disease phenotype, sensitive and efficient methods of detecting such environmental triggers are needed. Accordingly, provided herein, in some embodiments are sensors comprising a synthetic nucleic acid barcode, e.g., a modified nucleic acid barcode, for multiplexed sensing of disease.

One major obstacle to precision cancer diagnosis is accessing specific disease biomarkers to maximize the on-target signal generation in a real-time, noninvasive manner. It is well appreciated that microenvironmental characteristics such as extracellular matrix (ECM) alterations, stromal composition, or immune components exhibit critical determinants of metastatic dissemination broadly across cancers (Quail and Joyce, Nature medicine 2013, 19, 1423). As tumors start to invade, they alter the ECM architecture through aberrant proteolytic activities that could be leveraged as biomarkers. The Bhatia group recently described a class of injectable nanosensors that, in response to protease cleavage, release detectable reporters into urine as “synthetic biomarkers.” This technique combines the amplifying effects of enzymatic catalysis and renal enrichment to produce an ultra-sensitive detection signal. While the synthetic biomarkers have shown promise for robust tumor detection in animal models, improving their ability to achieve highly multiplexed monitoring of aberrant protease activities would greatly increase the pre-clinical and clinical applicability of this platform to distinguish diverse disease states.

The CRISPR (Clustered Regularly Interspaced Short Palindromic Repeats)-Cas (CRISPR-associated) adaptive immunity in bacteria and archaea has been widely deployed for gene-editing applications through the precise recognition of DNA/RNA molecules through complementarity to a guide RNA (Adli, Nature communications 2018, 9, 1911). A family of Cas enzymes, CRISPR-Cas12a (Cpf1), upon RNA-guided DNA binding, unleashes indiscriminate single-stranded DNA (ssDNA) cleavage activity (Chen et al., Science 2018, 360, 436). This target-activated, nonspecific single-stranded deoxyribonuclease (ssDNase) cleavage allows for rapid and specific nucleic acid detection, thereby providing a simple platform for molecular diagnostics. Although Cas12a and other Cas proteins (e.g., Cas13a, Cas13b, Cas14, and CasX) have been used for DNA or RNA diagnostics (Harrington et al., Science 2018, 362, 839; Liu et al., Nature 2019, 566, 218; Gootenberg et al., Science 2018, 360, 439), Cas proteins with indiscriminate nucleic acid cleavage activity have not been applied to in vivo disease-sensing and -monitoring.

As mentioned above, identification of genetic alterations is not sufficiently indicative of protein activity or of tissue microenvironments. Therefore, disease assays that rely on RNA and/or DNA in samples from subjects may not be indicative of the actual disease state. For example, a subject could have a genetic mutation, but the genetic mutation may not affect protein activity. Similarly, gene amplification may not always result in an increase in protein activity. Previous Cas-based diagnostic assays also require amplification of an endogenous biomarker (RNA or DNA), which can increase processing time. Assays that rely on endogenous biomarkers may have increased noise and higher false positive rates as compared to assays that rely on synthetic or orthogonal biomarkers. For example, samples could be contaminated with nucleic acids from end users or there may be off-target amplification of other nucleic acids of interest. Furthermore, without being bound by a particular theory, Cas proteins with indiscriminate nucleic acid cleavage activity may not previously have been used for in vivo applications due to the nonspecific degradation of unmodified nucleic acids by nucleases within the body.

In contrast, the sensors disclosed herein allow for noninvasive in vivo approaches that target and classify aggressive phenotypic features and monitor disease progression. In the in vivo sensing design, the diagnostic signals are triggered on-target through in vivo sensing of endogenous proteolytic activities in the tissue microenvironment and release barcoded reporters detectable in the urine. This noninvasive platform provides enriched real-time information and avoids intensive biopsies associated with transcriptomic and proteomic tools. To accurately reflect the complicated disease microenvironment, high-throughput nucleic acid barcoding enables a nucleic acid detection system, i.e., CRISPR-Cas-mediated, multiplexed, rapid, portable readout in resource limited settings. Not only can these novel sensors produce reporters for disease detection, they can be further engineered to guide therapeutics actions through longitudinal medical imaging. In some instances, the programmability of Cas proteins in combination with the barcodes disclosed herein allow for the generation of hundreds of orthogonal codes, which is challenging to attain with isobaric tags for use with mass-spectrometry. The methods described herein also obviate the need for rigorous assessment of instrumentation and data interpretation, which is often required with mass-encoded reporters. Without being bound by a particular theory, the sensors disclosed herein can be used to i) unveil new biology at the disease-specific microenvironment, ii) provide a completely noninvasive way to track disease progression and regression upon treatment(s), and iii) offer a pipeline for validating novel therapies.

The core technology described here leverages biological features (e.g., protease dysregulation), nanomaterial pharmacokinetics (e.g. tumor targeting, urinary secretion) and bio-orthogonality (e.g., reporters not present in living systems) to develop robust multiplex nanosensors. These degrees of precision are not readily amenable to endogenous biomarkers and may provide the ability to detect diseases such as cancer earlier than conventional diagnostics. In addition, clinical translation of diagnostic and therapeutic innovations has been restrained by the challenge of achieving disease site-specific delivery (Hunter et al., Nature reviews. Cancer 2006, 6, 141). In some embodiments, biologics sensitive to tumor specific factors were incorporated to enrich the delivery to sites of disease. In this integrated strategy, all three functional components, including a targeting module (nanobody), a stimuli responsive module (protease activated site) and a functionally effective module (diagnostic reporters) can be precisely interchanged tailoring the target specificities. Beyond cancer, dysregulated protease activities are implicated in number of pathologies such as fibrosis, thrombosis, infection and many more (Lin et al., ACS nano 2013, 7, 9001; Turk et al., Nature reviews. Drug discovery 2006, 5, 785; Shearer et al., The Journal of biological chemistry 2016, 291, 23188).

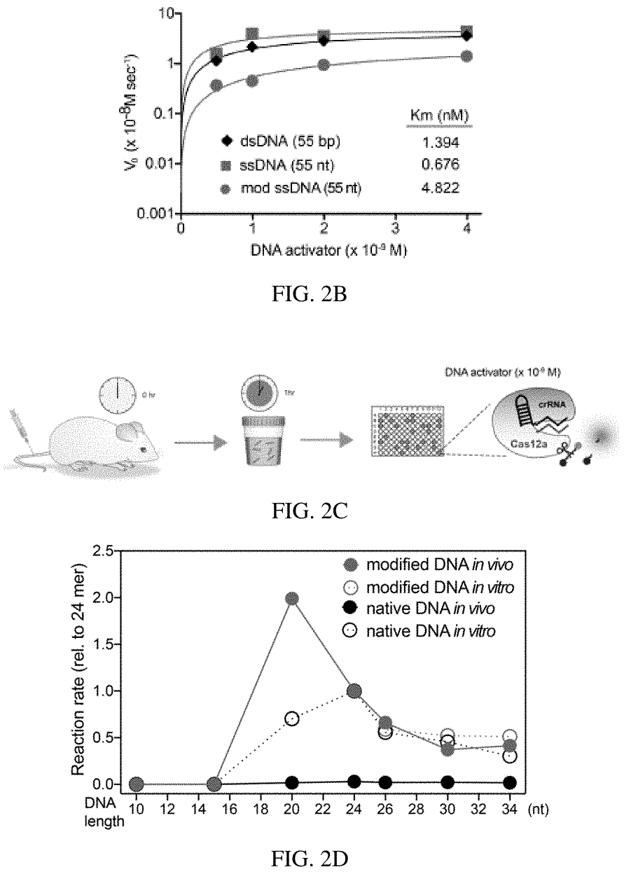

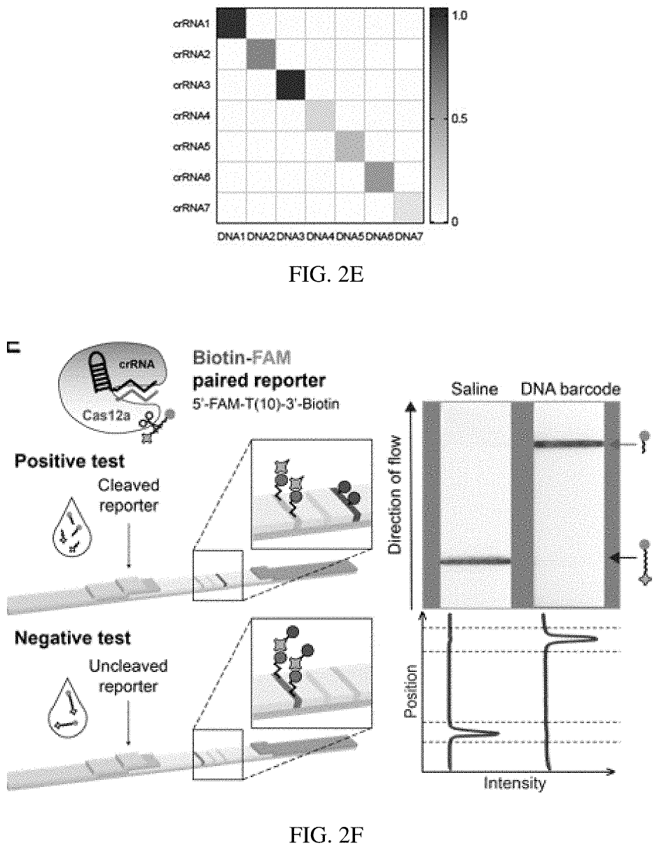

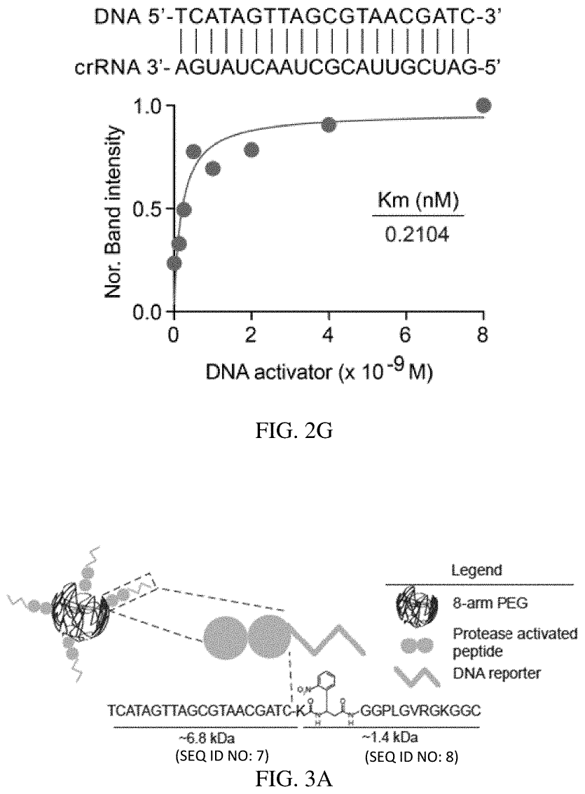

In some embodiments, the methods described herein provide a multiplexable readout of protease released signals that bridge translation to rapid point-of-care detection. In some embodiments, the in vivo sensors are barcoded with chemically-stabilized DNA to prevent nuclease degradation and immunostimulation, and to clear from the kidney (Dahlman et al., Proceedings of the National Academy of Sciences of the U.S. Pat. No. 2,017,114, 2060). In some embodiments, these barcodes are read in CRISPR-Cas based enzymatic assays. The CRISPR nuclease can be activated once it encounters its programmed nucleic target in unprocessed urine and cleaves a tagged construct that rapidly appears on a lateral flow paper strip. This detection step can happen within one hour at the point of care (POC), providing a new paradigm of cost-effective mapping of cancer proteolysis. Although the CRISPR-Cas-based enzymatic assays that have been used for direct pathogen detection, they have not been utilized for in vivo sensing of genetic disorders, which without being bound by a particular theory, may be due to the instability of nucleic acids in vivo. Here, it was demonstrated for the first time that pathological proteolytic activities can be leveraged to disassemble chemically stabilized DNA barcodes at the local disease site to guide understanding of the presence, progression or regression of diseases in situ. Unlike the previously reported mass-barcoded synthetic biomarker platform, application of DNA-barcoded in vivo sensors to monitor protease activity circumvents challenges including expanding multiplexing of the barcodes due to matrix complexity and the need for rigorous protocol validation (Kwong et al., Nature biotechnology 2013, 31, 63). In addition to the high-fidelity crRNA-DNA barcode binding for Cas12a activation (FIG. 2E), newly discovered Cas enzymes (i.e., Cas14) that exhibit programmed DNA destruction allow for highly specific SNP genotyping without the constraint of a PAM sequence (Chen et al., Science 2018, 360, 436; Harrington et al., Science 2018, 362, 839). Thus, without being bound by a particular theory, the pool of possible nucleic acid barcodes can be infinite (maximum 4²⁰ in theory) for a 20-mer oligonucleotide, covering all possible proteases (˜500 in human genome) responsive in vivo sensing requirements.

Accordingly, sensors that address many of these limitations are disclosed herein. Provided herein, in some embodiments, are methods to monitor noninvasively the complicated disease environment, leveraging high-throughput nucleic acid barcoding that allows for a rapid, CRISPR-Cas-mediated multiplexed, portable readout for use in resource-limited settings. The unique combination of responsive barcode-releasing and CRISPR techniques could substantially expand the multiplexing capabilities to empower disease classification at the POC.

### Nucleic Acid Barcodes

The sensors of the present disclosure comprise a nucleic acid barcode. The barcodes of the present disclosure may be double-stranded or single-stranded. The barcode may comprise ribonucleotides, and/or deoxyribonucleotides. In some embodiments, the barcode comprises single-stranded DNA (ssDNA), single-stranded RNA (ssRNA), double-stranded DNA (dsDNA) and/or double-stranded RNA (dsRNA).

In some embodiments, certain nucleotide modifications may be used that make a barcode into which they are incorporated more resistant to nuclease digestion than an unmodified barcode; barcodes comprising such modified nucleotides may survive intact for a longer time than unmodified oligonucleotides. It was found that phosphorothioate internucleotide linkages increased the nuclease resistance of nucleic acid barcodes, rendering them amenable for in vivo sensing. Surprisingly, despite barcodes comprising phosphorothioate internucleotide linkages exhibiting lower duplex melting temperatures, which may interfere, e.g., with Cas12a transcleavage activity, without being bound by a particular theory, the increase in nuclease resistance appears to be significant enough to make the linkages advantageous in barcodes and methods of the present disclosure. Accordingly, barcodes of the disclosure can be stabilized against nuclease degradation by the incorporation of a such a modification (e.g., a nucleotide modification).

A modified nucleic acid barcode comprises at least one nucleic acid modification. A modified nucleotide barcode may comprise a modified internucleoside linkage, a modified nucleotide, and/or a terminal modification. A modified nucleotide may comprise a modified sugar moiety and/or a modified base moiety. In some instances, a modified sugar moiety comprises a 2′-OH group modification and/or a bridging moiety. 2′-OH group modifications include 2′-O-Methyl (2′-O-Me), 2′-Fluoro (2′-F), and 2′-O-methoxy-ethyl (2′-O-MOE or 2′-0-Methoxyethyl (2′-MOE)). In some instances, a nucleotide with a bridging moiety is a locked nucleic acid. Non-limiting examples of modified bases include deoxyuridine (dU), a 5-Methyl deoxyCytidine (5-methyl dC), and an inverted dT.

Non-limiting examples of internucleoside linkage modifications include phosphorothioate (PS), boranophosphate, phosphoramidate, phosphorodiamidate morpholino (PMO), and thiophosphoramidate.

A barcode may be modified at the 5′ end, the 3′ end, or a combination thereof. In some embodiments, the terminal modification is a 5′ terminal modification phosphate modification (e.g., 5′-(E)-vinyl-phosphonate (5-VP)). In some embodiments, a barcode comprises a terminal phosphosphorylation (e.g., a 5′-phosphorylation and/or a 3′-phosphorylation).

A barcode may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different nucleic acid modifications. For example, a barcode may comprise an internucleoside linkages modification and a nucleotide with a modified base. For example, a barcode may comprise an internucleoside linkage modification and a nucleotide with a modified sugar. In some embodiments, a barcode may comprise two different internucleoside modifications. In some embodiments, all internucleoside linkages in a barcode may be modified. In some embodiments, a barcode comprises a phosphorothioate linkage and a 2′ O-methyl base. In some embodiments, a barcode comprises a phosphorothioate linkage and a locked nucleic acid.

In some instances, a barcode is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, a barcode comprises at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100 nucleotides in length.

In some instances, a barcode between 5-30, 10-30, 15-30, 20-30, 5-50, 10-50, 10-40, 20-40, 20-50, 30-50, 10-100, 1-100, 5-100, 5-10, 15-40, 60-80, or 40-50 nucleotides in length. In some embodiments, the barcode is 70 nucleotides in length.

In some embodiments, a barcode comprises a sequence with at most 1, at most 2, at most 3, at most 4, at most 5, at most 6, at most 7, at most 8, at most 9, or at most 10 positions of difference relative to a sequence selected from SEQ ID NOs: 15-49 or a sequence in Table 11. As a non-limiting example, a barcode may comprise a sequence with at most 1, at most 2, at most 3, at most 4, at most 5, at most 6, at most 7, at most 8, at most 9, or at most 10 nucleotide substitutions, deletions, insertions, or a combination thereof relative to a barcode sequence disclosed herein. In some instances, a barcode may comprise at most 1, at most 2, at most 3, at most 4, at most 5, at most 6, at most 7, at most 8, at most 9, or at most 10 modifications relative to a barcode sequence disclosed herein. In some embodiments, a barcode comprises a sequence that is at least 70%, at least 80%, at least 85%, at least 90%, at least 95%, at least 96%, at least 97%, at least 98%, at least 99%, or 100% identical to a sequence selected from SEQ ID NOs: 15-49 or a sequence in Table 11.

In some embodiments, the modified nucleic acid barcode has a molecular weight of 3-20, 3-15, 3-10, 3-8, 3-5, 5-20, 5-15, 5-10, 5-8, 8-20, 8-15, 8-10, 10-20, 10-15, or 15-20 kilodaltons (kDa). Without being bound by a particular theory, the molecular weight of a barcode may be a relevant design consideration in vivo, as nucleic acid barcodes may undergo a single-exponential concentration decay (e.g., due to circulating non-specific nucleases) after intravenous injection followed by size-dependent renal filtration from the blood.

### Cas-Based Nucleic Acid Detection Systems

In some embodiments, the modified nucleic acids that have been released from a sensor are detected using a Cas-based nucleic acid detection system (i.e.; a CRISPR-Cas based assay). A Cas system, CRISPR-Cas system or CRISPR system as used in herein generally refers to proteins, nucleic acids, or other components involved in the expression of or targeting the activity of CRISPR-associated (“Cas”) genes. Components of a CRISPR-Cas system include sequences encoding a Cas protein, tracr (trans-activating CRISPR) RNA sequences, and guide sequences. A guide sequence comprises at least a nucleic acid sequence that is complementary to a target sequence of interest. In some embodiments, the nucleic acid sequence that is complementary to a target sequence of interest is referred to as a CRISPR RNA (crRNA). A guide sequence may be a single guide RNA (sgRNA) (chimeric RNA) that comprises both a nucleic acid sequence that is complementary to a target sequence of interest and a tracr. Certain Cas proteins including Cas12a and Cas13a do not require a tracr. In some instances, a guide sequence does not comprise a tracr. See, e.g., Murugan et al., Mol Cell. 2017 Oct. 5; 68(1):15-25. In some embodiments, a Cas protein comprises a sequence that is at least 70%, at least 80%, at least 85%, at least 90%, at least 95%, at least 96%, at least 97%, at least 98%, at least 99%, or 100% identical to SEQ ID NO: 73.

A crRNA sequence may comprise one or more modifications disclosed herein. A modified crRNA may comprise at least one nucleic acid modification. A crRNA may comprise a modified internucleoside linkage, a modified nucleotide, and/or a terminal modification. A modified nucleotide may comprise a modified sugar moiety and/or a modified base moiety. In some instances, a modified sugar moiety comprises a 2′-OH group modification and/or a bridging moiety. 2′-OH group modifications include 2′-O-Methyl (2′-0-Me), 2′-Fluoro (2′-F), and 2′-O-methoxy-ethyl (2′-O-MOE or 2′-O-Methoxyethyl (2′-MOE)). In some instances, a nucleotide with a bridging moiety is a locked nucleic acid. Non-limiting examples of modified bases include deoxyuridine (dU), a 5-Methyl deoxyCytidine (5-methyl dC), and an inverted dT.

Non-limiting examples of internucleoside linkage modifications include phosphorothioate (PS), boranophosphate, phosphoramidate, phosphorodiamidate morpholino (PMO), and thiophosphoramidate.

A crRNA may be modified at the 5′ end, the 3′ end, or a combination thereof. In some embodiments, the terminal modification is a 5′ terminal modification phosphate modification (e.g., 5′-(E)-vinyl-phosphonate (5-VP)). In some embodiments, a barcode comprises a terminal phosphosphorylation (e.g., a 5′-phosphorylation and/or a 3′-phosphorylation).

A crRNA may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 different nucleic acid modifications. For example, a crRNA may comprise an internucleoside linkages modification and a nucleotide with a modified base. For example, a crRNA may comprise an internucleoside linkage modification and a nucleotide with a modified sugar. In some embodiments, a crRNA may comprise two different internucleoside modifications. In some embodiments, all internucleoside linkages in a crRNA may be modified. In some embodiments, a crRNA comprises a phosphorothioate linkage and a 2′ O-methyl base. In some embodiments, a crRNA comprises a phosphorothioate linkage and a locked nucleic acid.

In some embodiments, a crRNA comprises a sequence with at most 1, at most 2, at most 3, at most 4, at most 5, at most 6, at most 7, at most 8, at most 9, or at most 10 positions of difference relative to a sequence selected from SEQ ID NOs: 9-14 or a sequence in Table 10. As a non-limiting example, a crRNA may comprise a sequence with at most 1, at most 2, at most 3, at most 4, at most 5, at most 6, at most 7, at most 8, at most 9, or at most 10 nucleotide substitutions, deletions, insertions, or a combination thereof relative to a barcode sequence disclosed herein. In some instances, a crRNA may comprise at most 1, at most 2, at most 3, at most 4, at most 5, at most 6, at most 7, at most 8, at most 9, or at most 10 modifications relative to a crRNA disclosed herein. In some embodiments, a barcode comprises a sequence that is at least 70%, at least 80%, at least 85%, at least 90%, at least 95%, at least 96%, at least 97%, at least 98%, at least 99%, or 100% identical to a sequence selected from SEQ ID NOs: 9-14 or a sequence in Table 10.

A Cas-based nucleic acid detection system uses a Cas protein and a guide sequence that comprises a sequence that is complementary to a target sequence of interest to detect the target sequence. A Cas-based nucleic acid detection system often further comprises a reporter (e.g., a reporter with a sequence that can be cleaved by an activated Cas. Any Cas protein that, when activated, is capable of non-specific trans cleavage of a nucleic acid can be used with the methods described herein. Such Cas proteins are activated when a sequence comprising a CRISPR RNA binds to an “activator” sequence that comprises a sequence that is complementary to a sequence in the CRISPR RNA. In the assays described herein, the activator sequence is a nucleic acid barcode. In some embodiments, a nucleic acid barcode is single-stranded. In some embodiments, a nucleic acid barcode is double-stranded. In some embodiments, a nucleic acid barcode comprises a protospacer adjacent motif (PAM), which is recognized by the Cas protein. In some embodiments, the PAM sequence is 5′-TTN-3′. In some instances, the PAM sequence is 5′-TTTN-3.′ As a non-limiting example, a double-stranded nucleic acid barcode may comprise a PAM sequence that is located at the 5′ end of the nucleic acid barcode on the strand of the double-stranded nucleic acid that does not directly hybridize with the CRISPR RNA (the non-complementary strand). In some embodiments, a nucleic acid barcode does not comprise a PAM motif, which is recognized by the Cas protein. In some embodiments, a single-stranded nucleic acid barcode does not comprise a PAM motif.

As used herein, non-specific trans cleavage in reference to Cas protein activity refers to cleavage of a nucleic acid that is separate (unlinked) to the activator sequence and that does not comprise a sequence that is complementary to the CRISPR RNA used to target Cas protein. Cas proteins can be activated by binding a crRNA. Non-limiting examples of Cas proteins that, when activated, is capable of non-specific trans cleavage of a nucleic acid include a type V Cas protein, a type VI Cas protein, a Cas14, a CasX, a CasZ, or a CasY. Type V Cas protein include Cas12 proteins (e.g., Cpf1 (Cas12a), C2c1 (Cas12b), Cas12c, Cas12d, and Cas12e). Type VI Cas proteins include Cas13a and Cas13b. In some embodiments, a Cas proteins that, when activated, is capable of non-specific trans cleavage of a nucleic acid is a Cas13 protein. Non-limiting examples of Cas13 proteins include Cas13a, Cas13b, Cas13c, and Cas13d. Trans cleavage of a nucleic acid sequence can also be achieved using a combination of Cas proteins with auxiliary CRISPR-associated enzymes (e.g., Cas13 and Csm6, see, e.g., Gootenberg et al., Science. 2018 Apr. 27; 360(6387):439-444). Additional Cas proteins may be found for example in Harrington et al., Science 2018, 362, 839; Liu et al., Nature 2019, 566, 218; Gootenberg et al., Science 2018, 360, 439; U.S. Pat. No. 10,253,365; WO2019126762; WO2017120410; WO2019089820; WO2019089804; WO2019126716; WO2019148206; and WO2019126577, which is each hereby incorporated by reference only for the purpose of providing examples of Cas proteins that may be used to detect a nucleic acid barcode of the present disclosure.

Once activated, a Cas protein may be used to cleave a reporter sequence. In some instances, a reporter comprises at least two ligands that are connected by a linker. In some embodiments, the ligands fluorescently quench each other when linked and are de-quenched upon the cleavage of the linker. In some embodiments, the ligands are self-quenching. In some embodiments, a reporter comprises a fluorophore and a quencher of the fluorophore. As a non-limiting example, a reporter may comprise a FAM fluorophore and a CPQ-2 quencher separated by a nucleic acid sequence linker. In some embodiments, the reporter comprises a nucleic acid sequence with at least one modification (e.g., a modified base, backbone modification, a sugar modification, and/or a terminal modification). In some embodiments, the reporter comprises a single-stranded nucleic acid sequence. In some embodiments, the reporter comprises a double-stranded nucleic acid sequence. In some embodiments, a double-stranded nucleic acid sequence is used with a Cas12 (e.g., Cas12a).

In some embodiments, the reporter comprises a nucleic acid linker that links two different ligands that can each be recognized by a different antibody. In some embodiments, a lateral flow assay is used to detect the presence of a cleaved reporter. Lateral flow assays (LFA), also referred to herein as paper test strip assays, have historically been used for pregnancy tests. Any suitable ligands that are known in the art may be used with the LFA. An additional advantage of LFAs is that they do not require laboratory infrastructure. The assay is automated on the test strip, only requiring the user to apply sample to the sample pad, and the results can be read with the naked eye by inspection of a distinct colored stripe. For these reasons LFAs can be used in almost any setting. In the developed world, one potential implementation includes an injection of the biomarker nanoparticles at the clinic and then measurement by the patient at home later. LFAs, or rapid diagnostic tests RDT, have been developed for a number of diseases, including malaria and AIDS. For much of the developing world, however, the burden of infectious diseases is falling, while non-communicable diseases, such as cancer, are increasing. Unfortunately, LFAs for many diseases remain elusive due to the low level of endogenous biomarkers. In some embodiments, the methods of the invention, using an LFA to detect a reporter that is cleaved in the presence of a synthetic nucleic acid barcode that is released in the presence of an in vivo environmental trigger, provides a unique opportunity to diagnose diseases including cancer significantly earlier in places, like rural India and China, where a lack of medical infrastructure would otherwise make early diagnosis intractable. As a non-limiting example, a reporter comprising two different ligands may be used in combination with a LFA. The LFA may comprise a first region with an antibody that recognizes one of the ligands present on the reporter and a second region with an antibody that recognizes the other ligand present on the reporter. If the nucleic acid barcode (“activator” sequence) is present in a sample, a nucleic acid barcode comprising a sequence that is complementary to the CRISPR RNA sequence will activate the nucleic acid cleavage activity of the Cas protein. The activated Cas protein can then cleave the nucleic acid reporter. In a LFA, an uncleaved reporter will predominantly accumulate at the first region of the LFA. A cleaved reporter can be recognized at the second region. A labeled antibody can then be used to detect any bound cleaved or uncleaved reporters generating one or more bands on the LFA.

Aspects of the present disclosure also provide a LFA device that can be used to a reporter that has been released from the device. The device may comprise the Cas-based nucleic acid detection system comprising a crRNA sequence that comprises a guide sequence that is complementary to a sequence in the modified nucleic acid barcode; a Cas protein; and a reporter that comprises a first ligand that is connected to a second ligand through a single-stranded nucleic acid linker, wherein the single-stranded nucleic acid linker is not complementary to the guide sequence. A sample from a subject who has been administered a sensor described herein may be contacted with a CRISPR-Cas system disclosed herein. As a non-limiting example, a sample from a subject who has been administered a sensor described herein may be contacted with a LFA device disclosed herein.

In some embodiments, a CRISPR-Cas system is incubated for at least 1 minute, for at least 5 minutes, for at least 10 minutes, for at least 20 minutes, for at least 30 minutes, for at least 40 minutes, for at least 50 minutes, for at least an hour, for at least 1.5 hours, for at least 2 hours, for at least 2.5 hours, for at least 3 hours, for at least 4 hours, or for at least 5 hours with a sample obtained from a subject that has been administered a sensor described herein. In some embodiments, a CRISPR-Cas system is incubated for about 1-3 hours, i.e., about 1 hour or about 3 hours. Without being bound by a particular theory, the incubation time may be adjusted depending on the amount of one or more components of the Cas-based nucleic acid detection systems (e.g., the amount of Cas enzyme, the amount of crRNA, and/or the amount of reporter used).

### Scaffolds

The scaffold may serve as the core of the sensor (e.g., nanosensor). A purpose of the scaffold is to serve as a platform for the environmentally-responsive linker and enhance delivery of the sensor to tissue (e.g., disease tissue) in a subject. As such, the scaffold can be any material or size as long as it can enhance delivery and/or accumulation of the sensors to a tissue in a subject. Preferably, the scaffold material is non-immunogenic, i.e. does not provoke an immune response in the body of the subject to which it will be administered. Non-limiting examples of scaffolds, include, for instance, compounds that cause active targeting to tissue, cells or molecules (e.g., targeting of sensors to a tissue), microparticles, nanoparticles, aptamers, peptides (RGD, iRGD, LyP-1, CREKA, etc.), proteins, nucleic acids, polysaccharides, polymers, antibodies or antibody fragments (e.g., herceptin, cetuximab, panitumumab, etc.) and small molecules (e.g., erlotinib, gefitinib, sorafenib, etc.).

In some embodiments, the scaffold comprises a protein. For example, the scaffold may comprise a biotin-binding protein (e.g., avidin). Exemplary avidin proteins include, but are not limited to avidin, streptavidin, NeutrAvidin, and CaptAvidin.

In some embodiments, the scaffold has a diameter (e.g., hydrodynamic diameter) between 1 and 10 nm, between 2.5 and 10 nm, between 3 and 10 nm, between 5 and 10 nm, between 6 and 10 nm, between 7 and 10 nm, between 8 and 10 nm, between 7 and 8 nm, between 9 and 10 nm, between 10 nm and 20 nm, or between 20 nm and 30 nm. In some instances, a scaffold has a diameter of 8 nm. In some embodiments, the scaffold has a diameter that is greater than 5 nm. In some embodiments, the scaffold is at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, at least 200 nm, at least 300 nm, at least 400 nm, at least 500 nm, at least 600 nm, at least 700 nm, at least 800 nm, at least 900 nm, or at least 1,000 nm.

In some aspects, the disclosure relates to the discovery that delivery to a tissue in a subject is enhanced by sensors having certain polymer scaffolds (e.g., poly(ethylene glycol) (PEG) scaffolds). Polyethylene glycol (PEG), also known as poly(oxyethylene) glycol, is a condensation polymer of ethylene oxide and water having the general chemical formula HO(CH₂CH₂O)[n]H. Generally, a PEG polymer can range in size from about 2 subunits (e.g., ethylene oxide molecules) to about 50,000 subunits (e.g., ethylene oxide molecules. In some embodiments, a PEG polymer comprises between 2 and 10,000 subunits (e.g., ethylene oxide molecules).

A PEG polymer can be linear or multi-armed (e.g., dendrimeric, branched geometry, star geometry, etc.). In some embodiments, a scaffold comprises a linear PEG polymer. In some embodiments, a scaffold comprises a multi-arm PEG polymer. In some embodiments, a multi-arm PEG polymer comprises between 2 and 20 arms. Multi-arm and dendrimeric scaffolds are generally described, for example by Madaan et al. J Pharm Bioallied Sci. 2014 6(3): 139-150.

Additional polymers include, but are not limited to: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride and polystyrene.

Examples of non-biodegradable polymers include ethylene vinyl acetate, poly(meth) acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as algninate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. In some embodiments the polymers are polyesters, polyanhydrides, polystyrenes, polylactic acid, polyglycolic acid, and copolymers of lactic and glycoloic acid and blends thereof.

PVP is a non-ionogenic, hydrophilic polymer having a mean molecular weight ranging from approximately 10,000 to 700,000 and the chemical formula (C₆H₉NO)[n]. PVP is also known as poly[1-(2-oxo-1-pyrrolidinyl)ethylend], Povidone™, Polyvidone™, RP 143™, Kollidon™, Peregal ST™, Periston™, Plasdone™, Plasmosan™, Protagent™ Subtosan™, and Vinisil™. PVP is non-toxic, highly hygroscopic and readily dissolves in water or organic solvents.

Polyvinyl alcohol (PVA) is a polymer prepared from polyvinyl acetates by replacement of the acetate groups with hydroxyl groups and has the formula (CH₂CHOH)[n]. Most polyvinyl alcohols are soluble in water.

PEG, PVA and PVP are commercially available from chemical suppliers such as the Sigma Chemical Company (St. Louis, Mo.).

In certain embodiments the polymer may comprise poly(lactic-co-glycolic acid) (PLGA).

In some embodiments, a scaffold (e.g., a polymer scaffold, such as a PEG scaffold) has a molecular weight equal to or greater than 40 kDa. In some embodiments, a scaffold is a particle (e.g., an iron oxide nanoparticle, IONP) that is between 10 nm and 50 nm in diameter (e.g. having an average particle size between 10 nm and 50 nm, inclusive). In some embodiments, a scaffold is a high molecular weight protein, for example an Fc domain of an antibody.

In some embodiments, a scaffold comprises a particle. In some embodiments, a scaffold is a particle. As used herein the term “particle” includes nanoparticles as well as microparticles. Nanoparticles are defined as particles of less than 1.0 μm in diameter. A preparation of nanoparticles includes particles having an average particle size of less than 1.0 μm in diameter. Microparticles are particles of greater than 1.0 μm in diameter but less than 1 mm. A preparation of microparticles includes particles having an average particle size of greater than 1.0 μm in diameter. The microparticles may therefore have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter is about 5 nm to about 500 nm. In other embodiments, the diameter is about 100 nm to about 200 nm. In other embodiment, the diameter is about 10 nm to about 100 nm.

In some embodiments, one or more types of polymers are formed into nanoparticles (e.g., for use as a scaffold). In some embodiments, a scaffold is a branched polymer. In some embodiments, a scaffold is a nanoparticle comprised of polymers, which may further comprise at least one functional group for attaching a modified nucleic acid barcode. In some embodiments, a scaffold is a nanoparticle comprised of polymers and the scaffold encapsulates a modified nucleic acid barcode.

A preparation of particles, in some embodiments, includes particles having an average particle size of less than 1.0 μm in diameter or of greater than 1.0 μm in diameter but less than 1 mm. The preparation of particles may therefore, in some embodiments, have a diameter of at least 5, at least 10, at least 25, at least 50, or at least 75 microns, including sizes in ranges of 5-10 microns, 5-15 microns, 5-20 microns, 5-30 microns, 5-40 microns, or 5-50 microns. A composition of particles may have heterogeneous size distributions ranging from 10 nm to mm sizes. In some embodiments the diameter is about 5 nm to about 500 nm. In other embodiments, the diameter is about 100 nm to about 200 nm. In other embodiments, the diameter is about 10 nm to about 100 nm.

The scaffold may be composed of a variety of materials including iron, ceramic, metallic, natural polymer materials (including lipids, sugars, chitosan, hyaluronic acid, etc.), synthetic polymer materials (including poly-lactide-coglycolide, poly-glycerol sebacate, etc.), and non-polymer materials, or combinations thereof.

The scaffold may be composed in whole or in part of polymers or non-polymer materials. Non-polymer materials, for example, may be employed in the preparation of the particles. Exemplary materials include alumina, calcium carbonate, calcium sulfate, calcium phosphosilicate, sodium phosphate, calcium aluminate, calcium phosphate, hydroxyapatite, tricalcium phosphate, dicalcium phosphate, tricalcium phosphate, tetracalcium phosphate, amorphous calcium phosphate, octacalcium phosphate, and silicates. In certain embodiments the particles may comprise a calcium salt such as calcium carbonate, a zirconium salt such as zirconium dioxide, a zinc salt such as zinc oxide, a magnesium salt such as magnesium silicate, a silicon salt such as silicon dioxide or a titanium salt such as titanium oxide or titanium dioxide.

A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d′Amore; and U.S. Pat. Nos. 4,806,621; 4,638,045 to Kohn; see also Langer, Acc. Chem. Res. 33:94, 2000; Langer, J. Control Release 62:7, 1999; and Uhrich et al., Chem. Rev. 99:3181, 1999; all of which are incorporated herein by reference).

The scaffold may be composed of inorganic materials. Inorganic materials include, for instance, magnetic materials, conductive materials, and semiconductor materials. In some embodiments, the scaffold is composed of an organic material (e.g., a biological material that enhances delivery of the sensor to a tissue of a subject).

In some embodiments, the scaffold is a porous particle. A porous particle can be a particle having one or more channels that extend from its outer surface into the core of the particle. In some embodiments, the channel may extend through the particle such that its ends are both located at the surface of the particle. These channels are typically formed during synthesis of the particle by inclusion followed by removal of a channel forming reagent in the particle.

The size of the pores may depend upon the size of the particle. In certain embodiments, the pores have a diameter of less than 15 microns, less than 10 microns, less than 7.5 microns, less than 5 microns, less than 2.5 microns, less than 1 micron, less than 0.5 microns, or less than 0.1 microns. The degree of porosity in porous particles may range from greater than 0 to less than 100% of the particle volume. The degree of porosity may be less than 1%, less than 5%, less than 10%, less than 15%, less than 20%, less than 25%, less than 30%, less than 35%, less than 40%, less than 45%, or less than 50%. The degree of porosity can be determined in a number of ways. For example, the degree of porosity can be determined based on the synthesis protocol of the scaffolds (e.g., based on the volume of the aqueous solution or other channel-forming reagent) or by microscopic inspection of the scaffolds post-synthesis.

The scaffold may be comprised of a plurality of particles which may be homogeneous for one or more parameters or characteristics. A plurality that is homogeneous for a given parameter, in some instances, means that particles within the plurality deviate from each other no more than about +/−10%, preferably no more than about +/−5%, and most preferably no more than about +/−1% of a given quantitative measure of the parameter. As an example, the particles may be homogeneously porous. This means that the degree of porosity within the particles of the plurality differs by not more than +/−10% of the average porosity. In other instances, a plurality that is homogeneous means that all the particles in the plurality were treated or processed in the same manner, including for example exposure to the same agent regardless of whether every particle ultimately has all the same properties. In still other embodiments, a plurality that is homogeneous means that at least 80%, preferably at least 90%, and more preferably at least 95% of particles are identical for a given parameter.

The plurality of particles may be heterogeneous for one or more parameters or characteristics. A plurality that is heterogeneous for a given parameter, in some instances, means that particles within the plurality deviate from the average by more than about +/−10%, including more than about +/−20%. Heterogeneous particles may differ with respect to a number of parameters including their size or diameter, their shape, their composition, their surface charge, their degradation profile, whether and what type of agent is comprised by the particle, the location of such agent (e.g., on the surface or internally), the number of agents comprised by the particle, etc. The disclosure contemplates separate synthesis of various types of particles which are then combined in any one of a number of pre-determined ratios prior to contact with the sample. As an example, in one embodiment, the particles may be homogeneous with respect to shape (e.g., at least 95% are spherical in shape) but may be heterogeneous with respect to size, degradation profile and/or agent comprised therein.

Scaffold size, shape and release kinetics can also be controlled by adjusting the scaffold formation conditions. For example, scaffold formation conditions can be optimized to produce smaller or larger scaffolds, or the overall incubation time or incubation temperature can be increased.

The scaffold may be formulated, for instance, into liposomes, virosomes, cationic lipids or other lipid based structures. The term “cationic lipid” refers to lipids which carry a net positive charge at physiological pH. Such lipids include, but are not limited to, DODAC, DOTMA, DDAB, DOTAP, DC-Chol and DMRIE. Additionally, a number of commercial preparations of cationic lipids are available. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA). A variety of methods are available for preparing liposomes e.g., U.S. Pat. Nos. 4,186,183, 4,217,344, 4,235,871, 4,261,975, 4,485,054, 4,501,728, 4,774,085, 4,837,028, 4,946,787; and PCT Publication No. WO 91/17424. The particles may also be composed in whole or in part of GRAS components. i.e., ingredients are those that are Generally Regarded As Safe (GRAS) by the US FDA. GRAS components useful as particle material include non-degradable food based particles such as cellulose.
The scaffold can serve several functions. As discussed above, it may be useful for targeting the product to a specific region, such as tissue. In that instance, it could include a targeting agent such as a glycoprotein, an antibody, or a binding protein. The term “antibody” encompasses whole antibodies (immunoglobulins having two heavy chains and two light chains), and antibody fragments. Antibody fragments include, but are not limited to, camelid antibodies, heavy chain fragments (VHH), Fab fragments, F(ab′)2 fragments, nanobodies (single-domain antibodies), and diabodies (bispecific/bivalent dimeric antibody fragments). In some embodiments, the antibodies are monoclonal antibodies. Monoclonal antibodies are antibodies that are secreted by a single B cell lineage. In some embodiments, the antibodies are polyclonal antibodies. Polyclonal antibodies are antibodies that are secreted by different B cell lineages. In some embodiments, the antibodies are chimeric antibodies. Chimeric antibodies are antibodies made by fusing the antigen binding region (variable domains of the heavy and light chains, VH and VL) from one species (e.g., mouse) with the constant domain from another species (e.g., human). In some embodiments, the antibodies are humanized antibodies. Humanized antibodies are antibodies from non-human species whose protein sequences have been modified to increase their similarity to antibody variants produced naturally in humans. In some embodiments, the antibodies are fusion antibodies (e.g., fusion of VHH or other antibody fragments to other protein types).

In some embodiments, the antibody is a single-domain antibody (nanobody). In some embodiments, a nanobody is capable of binding a membrane protein that can be used to distinguish a healthy cell and a diseased cell. In some embodiments, the diseased cell is a cancer cell. In some instances, a nanobody is a fragment of an existing antibody. For example, a nanobody may consist of a variable domain (VH) of a heavy-chain antibody or of a conventional immunoglobulin. Non-limiting examples of nanobodies may be found in Zuo et al., BMC Genomics. 2017 Oct. 17; 18(1):797 and WO2012042026. In some instances, the nanobody is a c-Met nanobody, e.g., Clone 4E09 from WO2012042026 (SEQ ID NO: 73). In some instances, a scaffold comprises a sequence that is at least 70%, at least 80%, at least 85%, at least 90%, at least 95%, at least 96%, at least 97%, at least 98%, at least 99%, or 100% identical to SEQ ID NO: 71.

In some embodiments, a nanobody is capable of binding to a tumor antigen. In some embodiments, a tumor antigen is a membrane protein. Non-limiting tumor antigens are shown in Table 1. See also, e.g., Holland-Frei Cancer Medicine. Kufe et al., 6th edition. (2003). Non-limiting examples of nanobodies targeting tumor antigens are provided in Table 2. See also, e.g., Chakravarty et al., Theranostics. 2014 Jan. 29; 4(4):386-98.

TABLE 1
Non-limiting examples of tumor antigens.

| Example Antigen | Cancer Histology |
|---|---|
| 9D7 | RCC |
| BAGE family | Multi |
| BING-4 | Melanoma |
| Breast cancer | Breast, ovarian carcinoma |
| antigen (BRCA)1/2 | |
| CAGE family | Multi |
| Calcium-activated | Lung carcinoma |
| chloride channel 2 | |
| Cyclin-dependent | Multi |
| kinase-4 (CDK4) | |
| Carcino-embryonic | Colorectal carcinoma |
| antigen (CEA) | |
| Chronic | CML |
| myelogenous | |
| leukemia (antigen) | |
| 66 (CML66) | |
| Cyclin-B1 | Multi |
| Epithelial cell | Breast carcinoma |
| adhesion molecule | |
| (Ep-CAM) | |
| EphA3 | Multi |
| Fibronectin | Multi |
| GAGE family | Multi |
| Gp100/pme117 | Melanoma |
| Her2/neu | Multi |
| Human papilloma | Cervical carcinoma |
| virus (HPV) E6, E7 | |
| Immunoglobulin | B, T leukemia, lymphoma, |
| (Ig), T cell receptor | myeloma |
| (TCR) | |
| Immature laminin | RCC |
| receptor | |
| MAGE family | Multi |
| Melanoma antigen | Melanoma |
| recognized by T | |
| cells (MART)-2 | |
| Melanocortin-1- | Melanoma |
| receptor (MC1R) | |
| Melan-A/Melanoma | Melanoma |
| antigen recognized | |
| by T cells (MART)-1 | |
| Mesothelin | Ductal pancreatic carcinoma |
| MUC1 | Ductal carcinoma, RCC |
| NY-ESO-1/LAGE-1 | Multi |
| P. polypeptide | Melanoma |
| p53 | Multi |
| PRAME | Multi |
| Prostate-pecific | Prostate |
| antigen | |
| Ras | Multi |
| SAGE family | Multi |
| Stomach cancer- | Colorectal carcinoma |
| associated protein | |
| tyrosine | |
| phosphatase-1 | |
| (SAP-1) | |
| SSX-2 | Melanoma, Multi |
| Survivin | Multi |
| Tumor antigen-72 | Prostate carcinoma |
| (TAG)-72 | |
| Telomerase | Multi |
| transforming growth | Colorectal carcinoma |
| factor-β receptor II | |
| (TGF-βRII) | |
| Tyrosinase-related | Melanoma |
| protein (TRP)-1/-2 | |
| Tyrosinase | Melanoma |
| XAGE family | Multi |
| β-catenin | Melanoma, Prostate, HCC |

TABLE 2
Non-limiting examples of tumor antigens.

| Target | Nanobody | Disease model | References |
|---|---|---|---|
| EGFR | 8B6 | Epidermoid | Huang et al., Mol |
| | | carcinoma, | Imaging Biol. 2008; |
| | | prostate | 10: 167-75 |
| | | carcinoma | |
| | 7C12 | Epidermoid | GainKam et al., |
| | | carcinoma | Contrast Media Mol |
| | | | Imaging. 2011; 6: |
| | | | 85-92 |
| | 7D12 | Epidermoid | Vosjan et al., Eur |
| | | carcinoma | J Nucl Med Mol |
| | | | Imaging. 2011; 38: |
| | | | 753-63. |
| HER-2 | 2Rs15d | Colon | Vaneycken et al., |
| | | carcinoma, | FASEB J. 2011; 25: |
| | | Breast cancer, | 2433-46 |
| | | Ovarian cancer | |
| | 2Rs15d | Ovarian cancer | Xavier et al., J |
| | | | Nucl Med. 2013; 54: |
| | | | 776-84. |
| | 11A4 | Breast cancer | Kijanka et al., Eur |
| | | | J Nucl Med Mol |
| | | | Imaging. 2013; 40: |
| | | | 1718-29 |
| HGF | 1E2 and | Glioblastoma | Vosjan et al., Mol |
| | 6E10 | | Cancer Ther. 2012; |
| | | | 11: 1017-25 |
| MMR | α-MMR | Mammary | Movahedi et al., |
| | | adenocarcinoma, | Cancer Res. 2012; |
| | | Lewis lung | 72: 4165-77. |
| | | carcinoma | |
| | | Rheumatoid | Put et al., J Nucl |
| | | arthritis | Med. 2013; 54: |
| | | | 807-14. |
| VCAM-1 | cAbVCAM1-5 | Atherosclerosis | Broisat et al., |
| | | | Circ Res. 2012; |
| | | | 110: 927-37. |
| CEA | CEA1 | Colon | Cortez-Retamozo |
| | | adenocarcinoma | el al., Current |
| | | | Radiopharm. 2008; |
| | | | 1: 37-41. |

In some embodiments, the membrane protein is a receptor tyrosine kinase. Non-limiting examples of receptor tyrosine kinases include c-Met, epidermal growth factor receptor (EGFR), fibroblast growth factor receptor (FGFR), vascular endothelial growth factor receptor (VEGFR), human epidermal growth factor receptor 2 (HER2), human epidermal growth factor receptor 3 (HER3 or ERBB3), and insulin-like growth factor 1 receptor.

In some embodiments, an antibody, including a nanobody, may be linked to another moiety (e.g., an enzyme substrate that is connected to a nucleic acid barcode) using any suitable method known in the art including alkyne-azide cycloaddition, lysine amide coupling, and cysteine-based conjugation in which one or more cysteine residue in an antibody is conjugated to a thiol-reactive functional group on the nucleic acid barcode. See, e.g., Tsuchikama et al., Protein Cell. 2018 January; 9(1):33-46. Other non-limiting examples of bioconjugation include use of DBCO, BCN, Tetrazine, TCO, APN and PTAD with PEG spacers. In some embodiments, a nucleic acid barcode is linked through the carboxy-terminus to an antibody. See, e.g., Example 1 below. In some embodiments, the linker is an enzyme substrate. In some embodiments, an enzyme-cleavable linker is linked to a barcode through an internal UV-sensitive residue (photocleavable residue). As an example, the internal UV-sensitive residue may be 3-amino-3-(2-nitrophenyl)propionic acid. In some embodiments, a moiety used for linking a barcode, enzyme substrate, or scaffold to another part of a sensor described herein may be included in the finished sensor. In other embodiments, a moiety (e.g., DBCO or azide) used for linking a barcode, enzyme substrate, or scaffold to another part of a sensor described herein is not included in the finished sensor (e.g., the moiety acts as a leaving group and/or facilitates conjugation chemistry). The barcodes of the present disclosure may or may not comprise a linking moiety. In some instances, the linking moiety is DBCO or azide.

Further, the size of the scaffold may be adjusted based on the particular use of the in vivo sensor. For instance, the scaffold may be designed to have a size greater than 5 nm. Particles, for instance, of greater than 5 nm are not capable of entering the urine, but rather, are cleared through the reticuloendothelial system (RES; liver, spleen, and lymph nodes). By being excluded from the removal through the kidneys any uncleaved sensor will not be detected in the urine during the analysis step. Additionally, larger particles can be useful for maintaining the particle in the blood or in a tumor site where large particles are more easily shuttled through the vasculature. In some embodiments the scaffold is 500 microns-5 nm, 250 microns-5 nm, 100 microns-5 nm, 10 microns-5 nm, 1 micron-5 nm, 100 nm-5 nm, 100 nm-10 nm, 50 nm-10 nm or any integer size range therebetween. In other instances the scaffold is smaller than 5 nm in diameter. In such instance, the sensor will be cleared into the urine. In some embodiments the scaffold is 1-5 nm, 2-5 nm, 3-5 nm, or 4-5 nm in diameter. Optionally, the scaffold may include a biological agent. In one embodiment, a biological agent could be incorporated in the scaffold or it may make up the scaffold. Thus, the compositions of the invention can achieve two purposes at the same time, the diagnostic methods and delivery of a therapeutic agent. In some embodiments, the biological agent may be an enzyme inhibitor. In that instance the biological agent can inhibit proteolytic activity at a local site and the modified nucleic acid barcode can be used to test the activity of that particular therapeutic at the site of action.

### Linkers

As used herein “linked” or “linkage” means two entities are bound to one another by any physicochemical means. Any linkage known to those of ordinary skill in the art, covalent or non-covalent, is embraced. Thus, in some embodiments the scaffold has a linker (e.g., environmentally-responsive linker) attached to an external surface, which can be used to link the modified nucleic acid barcode.

The in vivo sensors of the present disclosure comprise an environmentally-responsive linker that is located between the scaffold and the modified nucleic acid barcode. An environmentally-responsive linker, as used herein, is the portion of the sensor that changes in structure in response to an environmental trigger in the subject, causing the release of a modified nucleic acid barcode. Thus, an environmentally-responsive linker has two forms. The original form of the linker is attached to the scaffold and the modified nucleic acid barcode. When exposed to an environmental trigger the linker is modified in some way. For instance, it may be cleaved by an enzyme such that the modified nucleic acid barcode is released. Alternatively, it may undergo a conformational change which leads to release of the modified nucleic acid barcode.

In some embodiments, an environmentally-responsive linker is directly linking the modified nucleic acid barcode to the scaffold. In some embodiments, a scaffold comprises an environmentally-responsive linker that encapsulates a modified nucleic acid barcode.

An environmentally-responsive linker is a linker that is cleaved in response to an environmental trigger. Certain environmental triggers present in a disease microenvironments have been associated with disease. For example, environmental triggers include enzymes, light, pH, and temperature. An enzyme, as used herein refers to any of numerous proteins produced in living cells that accelerate or catalyze the metabolic processes of an organism. Enzymes act on substrates. The substrate binds to the enzyme at a location called the active site just before the reaction catalyzed by the enzyme takes place. Enzymes include but are not limited to proteases, glycosidases, lipases, heparinases, and phosphatases. In some instances, an environmental linker comprises a photolabile group, which may change conformation in response to light (e.g., to a particular wavelength of light).

In some embodiments, an environmentally-responsive linker is cleaved in response to the activity of an enzyme. In some embodiments, the enzyme is a protease. In some embodiments, the protease is a metalloprotease (e.g., a matrix metalloprotease), serine protease, aspartic protease, threonine protease, glutamic protease, asparagine peptide lyase, or a cysteine protease. In some instances, a cysteine protease is cathepsin B.

Dysregulated protease activities are implicated in a wide range of human diseases; including cancer, pulmonary embolism, inflammation, and infectious diseases, such as, bacterial infections, viral infections (e.g., HIV) and malaria. A sensor of the present disclosure may be used to detect an endogenous and/or an exogenous protease. An endogenous protease is a protease that is naturally produced by a subject (e.g., subject with a particular disease or a host with an infection). An exogenous protease is a protease that is not naturally produced by a subject and may be produced by a pathogen (e.g., a bacteria, a fungi, protozoa, or a virus). In some embodiments, a protease is only expressed by a subject (e.g., a human) and not by pathogen. In some embodiments, a protease is pathogen-specific and is only produced by a pathogen not by the pathogen's host.

Table 3 provides a non-limiting list of enzymes associated with (either increased or decreased with respect to normal) disease and in some instances, the specific substrate. Table 4 provides a non-limiting list of substrates associated with disease or other conditions. Numerous other enzyme/substrate combinations associated with specific diseases or conditions are known to the skilled artisan and are useful according to the invention.

TABLE 3
Non-limiting examples of disease-
associated enzymes and substrates.

| Disease | Enzyme | Substrate |
|---|---|---|
| Cancer | MMP | collagens, gelatin, |
| | | various ECM |
| | | proteins |
| Cancer | MMP-2 | type IV collagen and |
| | | gelatin |
| Cancer | MMP-9 | type IV and V |
| | | collagens and gelatin |
| Cancer | Kallikreins | kininogens, |
| | | plasminogen |
| Cancer | Cathepsins | broad spectrum of |
| | | substrates |
| Cancer | plasminogen | Plasminogen |
| | activator, tPA | |
| Cancer | Urokinase-type | Plasminogen |
| | plasminogen | |
| | activator, uPA | |
| | or PLAU | |
| Cancer | ADAM (A | various extracellular |
| | Diseintegrin And | domains of |
| | Metalloprotease, | transmembrane |
| | also MDC, | proteins |
| | Adamalysin) | |
| Pancreatic | MMP-7 | various, e.g. |
| carcinoma | | collagen 18, FasL, |
| | | HLE, DCN, IGFBP- |
| | | 3, MAG, |
| | | plasminogen, other |
| | | MMPs |
| Pancreatic Cancer | ADAM9, ADAM15 | various extracellular |
| | | domains of |
| | | transmembrane |
| | | proteins |
| Prostate | Matriptase, a | unspecific, cleaves |
| adenocarcinoma | type II | after Lys or Arg |
| | transmembrane | residues |
| | serine protease | |
| Prostate cancer | Kallikrein 3 | kininogens, |
| | | plasminogen |
| Prostate cancer | ADAM15 | various extracellular |
| | | domains of |
| | | transmembrane |
| | | proteins |
| Ovarian carcinoma | Kallikrein 6 | kininogens, |
| | | plasminogen |
| Epithelial-derived | Matriptase, a | unspecific, cleaves |
| tumors (breast, | type II | after Lys or Arg |
| prostate, ovarian, | transmembrane | residues |
| colon, oral) | serine protease | |
| Ovarian Cancer | MMP-2, MMP-9, | type IV and V |
| | kallikrein-10 | collagens and |
| | (hk-10) | gelatin, kininogens, |
| | | plasminogen |
| Breast, gastric, | cathepsins B, | broad spectrum of |
| prostate cancer | L and D | substrates |
| Endometrial cancer | cathepsin B | unspecific cleavage |
| | | of a broad spectrum |
| | | of substrates without |
| | | clear sequence |
| | | specificity |
| esophageal | cathepsin B | unspecific cleavage |
| adenocarcinoma | | of a broad spectrum |
| | | of substrates without |
| | | clear sequence |
| | | specificity |
| Invasive cancers, | type II integral | |
| metastases | serine proteases | |
| | (dipeptidyl | |
| | peptidase IV | |
| | (DPP4/CD26), | |
| | seprase/fibroblast | |
| | activation protein | |
| | alpha (FAPalpha) | |
| | and related type | |
| | II transmembrane | |
| | prolyl serine | |
| | peptidases)) | |
| Invasive cancers, | Seprase | various ECM |
| metastases | | proteins |
| Viral Infections | | |
| All Retroviruses | viral protease | precursor GagPol |
| | | fusion |
| HIV | HIV protease (HIV | precursor Gag and |
| | PR, an aspartic | GagPol proteins |
| | protease) | |
| Hepatitis C | NS3 serine | viral precursor |
| | protease | polyprotein |
| Dengue | Dengue protease | autocleavage |
| | | (NS2B/NS3), |
| | | NS3/NS4A and |
| | | NS4B/NS5 cleavage |
| West Nile | NS2B/NS3pro | viral precursor |
| | | polyprotein |
| Bacterial Infections | | |
| Legionella spp. | zinc | Me-Arg-Pro-Tyr |
| | metalloprotease | |
| Meninogencephalitis | histolytic | |
| | cysteine protease | |
| Streptococcus | streptococcal | extracellular matrix, |
| pyogenes (Group | pyrogenic exotoxin | immunoglobulins, |
| A Streptococcus) | B (SpeB) | complement |
| | | components |
| Clostridium | Cwp84 | fibronectin, laminin, |
| difficile | | vitronectin and other |
| | | ECM proteins |
| Pseudomonas | lasA | Leu-Gly-Gly-Gly- |
| aeruginosa | | Ala |
| Pseudomonas | Large | Cleavage of peptide |
| aeruginosa | ExoProtease A | ligands on PAR1, |
| | | PAR2, PAR4 |
| | | (Protease-activated |
| | | receptor). See, e.g., |
| | | Kida et al, Cell |
| | | Microbiol. 2008 |
| | | July; 10(7): 1491-504. |
| Pseudomonas | protease IV | complement factors, |
| aeruginosa | | fibrinogen, |
| | | plasminogen (See, |
| | | e.g., Engel et al., |
| | | J Biol Chem. 1998 |
| | | Jul. 3; 273(27): |
| | | 16792-7). |
| Pseudomonas | alkaline protease | Complement factor |
| aeruginosa | | C2 (See, e.g., |
| | | Laarman et al., J |
| | | Immunol. 2012 Jan. |
| | | 1; 188(1): 386-93). |
| Additional Diseases | | |
| Alzheimer′s | BACE-1,2 (Alzheimer | β-amyloid |
| disease | secretase) | precursor protein |
| Stroke and recovery | MMP, tPA | |
| cardiovascular | Angiotensin | angiotensin I, |
| disease | Converting Enzyme | bradykinin |
| | (ACE) | |
| Atherosclerosis | cathepsin K, L, S | broad spectrum of |
| | | substrates |
| Arthritis | MMP-1 | triple-helical |
| | | fibrillar collagens |
| rheumatoid arthritis | thrombin | Osteopontin |
| Malaria | SUB1 | KITAQDDEES |
| osteoarthritis | thrombin | Osteopontin |
| osteoporosis/ | cathepsin K, S | broad spectrum of |
| osteoarthritis | | substrates |
| Arthritis, | Aggrecanase | aggrecans |
| inflammatory joint | (ADAMTS4, | (proteoglycans) |
| disease | ADAMTS11) | |
| thrombosis | factor Xa | Prothrombin |
| | (thrombokinase) | |
| thrombosis | ADAMTS13 | von Willebrand |
| | | factor (vWF) |
| thrombosis | plasminogen | Plasminogen |
| | activator, tPA | |
| Stress-induced | Prostasin | epithelial Na |
| Renal pressure | | channel subunits |
| natriuresis | | |

TABLE 4
Non-limiting examples of substrates associated
with disease and other conditions.

| DISEASE | TARGET SUBSTRATE | ENZYME |
|---|---|---|
| Inflammation | Interleukin 1 beta | MMP-2, MMP-3, MMP-9, |
| | | Trypsin, chymotrypsin, |
| | | pepsin, Lys-C, Glu-C, |
| | | Asp-N, Arg-C |
| Pituitary gland | IGFBP-3 | MMP-1, MMP-3, MMP-9, |
| dysfunction, | | Trypsin, chymotrypsin, |
| abnormal bone | | pepsin, Lys-C, Glu-C, |
| density, growth | | Asp-N, Arg-C |
| disorders | | |
| Cancer | TGF-beta | MMP-9, Trypsin, |
| | | chymotrypsin, pepsin, |
| | | Lys-C, Glu-C, Asp-N, |
| | | Arg-C |
| Cancer, | TNF | MMP-7, Trypsin, |
| autoimmune | | chymotrypsin, pepsin, |
| disease | | Lys-C, Glu-C, Asp-N, |
| | | Arg-C |
| Cancer, | FASL | MMP-7, Trypsin, |
| autoimmune | | chymotrypsin, pepsin, |
| disease | | Lys-C, Glu-C, Asp-N, |
| | | Arg-C |
| Wound healing, | HB-EGF | MMP-3, Trypsin, |
| cardiac disease | | chymotrypsin, pepsin, |
| | | Lys-C, Glu-C, Asp-N, |
| | | Arg-C |
| Pfeiffer | FGFR1 | MMP-2, Trypsin, |
| syndrome | | chymotrypsin, pepsin, |
| | | Lys-C, Glu-C, Asp-N, |
| | | Arg-C |
| Cancer | Decorin | MMP-2, MMP-3, MMP-7, |
| | | Trypsin, chymotrypsin, |
| | | pepsin, Lys-C, Glu-C, |
| | | Asp-N, Arg-C |
| Cancer | Tumor associated | Endoglycosidases |
| | carbohydrate | |
| | antigens | |
| Cancer | Sialyl Lewis^{a} | O-glycanase |
| Cancer | Sialyl Lewis^{X} | O-glycanase |
| Cancer/ | VEGF | Trypsin, chymotrypsin, |
| Rheumatoid | | pepsin, Lys-C, Glu-C, |
| Arthritis, | | Asp-N, Arg-C |
| pulmonary | | |
| hypertension | | |
| Cancer | EGF | Trypsin, chymotrypsin, |
| | | pepsin, Lys-C, Glu-C, |
| | | Asp-N, Arg-C |
| Cancer | IL2 | Trypsin, chymotrypsin, |
| | | pepsin, Lys-C, Glu-C, |
| | | Asp-N, Arg-C |
| Cancer | IL6 | Trypsin, chymotrypsin, |
| inflammation/ | | pepsin, Lys-C, Glu-C, |
| angiogenesis | | Asp-N, Arg-C |
| Cancer | IFN-γ | Trypsin, chymotrypsin, |
| | | pepsin, Lys-C, Glu-C, |
| | | Asp-N, Arg-C |
| Cancer | TNF-α | Trypsin, chymotrypsin, |
| inflammation/ | | pepsin, Lys-C, Glu-C, |
| angiogenesis, | | Asp-N, Arg-C |
| Rheumatoid | | |
| Arthritis | | |
| Cancer, | TGF-β | Trypsin, chymotrypsin, |
| Pulmonary | | pepsin, Lys-C, Glu-C, |
| fibrosis, | | Asp-N, Arg-C |
| Asthma | | |
| Cancer, | PDGF | Trypsin, chymotrypsin, |
| Pulmonary | | pepsin, Lys-C, Glu-C, |
| hypertension | | Asp-N, Arg-C |
| Cancer, | Fibroblast growth | Trypsin, chymotrypsin, |
| pulmonary | factor (FGF) | pepsin, Lys-C, Glu-C, |
| cystadenoma | | Asp-N, Arg-C |
| Cancer | Brain-derived | Trypsin, chymotrypsin, |
| | neurotrophic | pepsin, Lys-C, Glu-C, |
| | factor (BDNF) | Asp-N, Arg-C |
| Cancer | Interferon | Trypsin, chymotrypsin, |
| | regulatory factors | pepsin, Lys-C, Glu-C, |
| | (IRF-1, IRF-2) | Asp-N, Arg-C |
| Inhibitor of | MIF | Trypsin, chymotrypsin, |
| tumor | | pepsin, Lys-C, Glu-C, |
| suppressors | | Asp-N, Arg-C |
| Lymphomas/ | GM-CSF | Trypsin, chymotrypsin, |
| carcinomas, | | pepsin, Lys-C, Glu-C, |
| alveolar | | Asp-N, Arg-C |
| proteinosis | | |
| Cancer invasion | M-CSF | Trypsin, chymotrypsin, |
| | | pepsin, Lys-C, Glu-C, |
| | | Asp-N, Arg-C |
| Chemical | IL-12 | Trypsin, chymotrypsin, |
| carcinogenesis, | | pepsin, Lys-C, Glu-C, |
| multiple sclerosis, | | Asp-N, Arg-C |
| rheumatoid | | |
| arthritis, | | |
| Crohn′s disease | | |
| Natural Killer T | IL-15 | Trypsin, chymotrypsin, |
| cell leukemias, | | pepsin, Lys-C, Glu-C, |
| inflammatory | | Asp-N, Arg-C |
| bowel disease, | | |
| rheumatoid | | |
| arthritis | | |
| Cirrhosis | Tissue inhibitor | Trypsin, chymotrypsin, |
| | of MMPs (TIMPs) | pepsin, Lys-C, Glu-C, |
| | | Asp-N, Arg-C |
| Cirrhosis | Collagen I, III | MMP-1, MMP-8, Trypsin, |
| | | chymotrypsin, pepsin, |
| | | Lys-C, Glu-C, Asp-N, |
| | | Arg-C |
| Cirrhosis | Collagen IV, V | MMP-2, Trypsin, |
| | | chymotrypsin, pepsin, |
| | | Lys-C, Glu-C, Asp-N, |
| | | Arg-C |

Several of the enzyme/substrates described above are described in the following publications, all of which are incorporated herein in their entirety by reference: Parks, W. C. and R. P. Mecham (Eds): Matrix metalloproteinases. San Diego: Academic Press; 1998; Nagase, H. and J. F. Woessner, Jr. (1999) J. Biol. Chem. 274:21491; Ito, A. et al. (1996) J. Biol. Chem. 271:14657; Schonbeck, U. et al. (1998) J. Immunol. 161: 3340; Rajah, R. et al. (1999) Am. J. Cell Mol. Biol. 20:199; Fowlkes, J. L. et al. (1994) Endocrinology 135:2810; Manes, S. et al. (1999) J. Biol. Chem. 274:6935; Mira, E. et al. (1999) Endocrinology 140:1657; Yu, Q. and I. Stamenkovic (2000) Genes Dev. 14:163; Haro, H. et al. (2000) J. Clin. Invest. 105:143; Powell, C. P. et al. (1999) Curr. Biol. 9:1441; Suzuki, M. et al. (1997) J. Biol. Chem. 272:31730; Levi, E. et al. (1996) Proc. Natl. Acad. Sci. USA 93:7069; Imai, K. et al. (1997) Biochem. J. 322:809; Smith, M. M. et al. (1995) J. Biol. Chem. 270:6440; and Dranoff, G. (2004) Nat. Rev. Cancer 4: 11-22.

In some embodiments, a linker is a cleavable linker. In some embodiments, a cleavable linker is an enzyme cleavable linker. Non-limiting examples of enzyme cleavable linkers may also be found in WO2010/101628, entitled METHODS AND PRODUCTS FOR IN VIVO ENZYME PROFILING, which was filed on Mar. 2, 2010; WO2012/125808, entitled MULTIPLEXED DETECTION WITH ISOTOPE-CODED REPORTERS, which was filed on Mar. 15, 2012; WO2014/197840, entitled AFFINITY-BASED DETECTION OF LIGAND-ENCODED SYNTHETIC BIOMARKERS, which was filed on Jun. 6, 2014; WO2017/193070, entitled METHODS AND USES FOR REMOTELY TRIGGERED PROTEASE ACTIVITY MEASUREMENTS, which was filed on May 5, 2017; WO2017/177115, entitled METHODS TO SPECIFICALLY PROFILE PROTEASE ACTIVITY AT LYMPH NODES, which was filed on Apr. 7, 2017; WO2018/187688, entitled METHODS TO SPATIALLY PROFILE PROTEASE ACTIVITY IN TISSUE AND SECTIONS, which was filed on Apr. 6, 2018; WO2019/075292, entitled PROSTATE CANCER PROTEASE NANOSENSORS AND USES THEREOF, which was filed on Oct. 12, 2018; WO2019/173332, entitled INHALABLE NANOSENSORS WITH VOLATILE REPORTERS AND USES THEREOF, which was filed on Mar. 5, 2019; WO2020/068920, entitled LUNG PROTEASE NANOSENSORS AND USES THEREOF, which was filed on Sep. 25, 2019; WO2020/150560, entitled SENSORS FOR DETECTING AND IMAGING OF CANCER METASTASIS, which was filed on Jan. 17, 2020; and WO2020/081635, entitled RENAL CLEARABLE NANOCATALYSTS FOR DISEASE MONITORING, which was filed on Oct. 16, 2019, which is each herein incorporated by reference in its entirety.

In some embodiments, an enzyme substrate comprises a sequence that is at least 70%, at least 80%, at least 85%, at least 90%, at least 91%, at least 92%, at least 93%, at least 94%, at least 95%, at least 96%, at least 97%, at least 98%, at least 99%, or 100% identical to an amino acid sequence selected from SEQ ID NOs: 50-70. In some embodiments, an enzyme substrate comprises a sequence having no more than 1, 2, 3, 4, or 5 positions of difference relative to an amino acid sequence selected from SEQ ID NOs: 50-70. In some instances, an enzyme substrate present in a sensor does not further comprise a fluorophore. In some instances, an enzyme substrate does not further comprise a quencher. In some instances, an enzyme substrate does not further comprise a quencher or a fluorophore.

In some embodiments, an enzyme substrate comprises a sequence that is at least 70%, at least 80%, at least 85%, at least 90%, at least 91%, at least 92%, at least 93%, at least 94%, at least 95%, at least 96%, at least 97%, at least 98%, at least 99%, or 100% identical to an amino acid sequence selected from SEQ ID NOs: 50-54. In some embodiments, an enzyme substrate comprises a sequence having no more than 1, 2, 3, 4, or 5 positions of difference relative to an amino acid sequence selected from SEQ ID NOs: 50-54. In some instances, an enzyme substrate present in a sensor does not further comprise an azide moiety.

A disease microenvironment may have a pH that deviates from a physiological pH. Physiological pH may vary depending on the subject. For example, in humans, the physiological pH is generally between 7.3 and 7.4 (e.g., 7.3, 7.35, or 7.4). A disease microenvironment may have a pH that is higher (e.g., more basic) or lower (e.g., more acidic) than a physiological pH. As an example, acidosis is characterized by an acidic pH (e.g., pH of lower than 7.4, a pH of lower than 7.35, or a pH of lower than 7.3) and is caused by metabolic and respiratory disorders. Non-limiting examples of diseases associated with acidosis include cancer, diabetes, kidney failure, chronic obstructive pulmonary disease, pneumonia, asthma and heart failure. In some embodiments, an acidic pH induces cleavage of an environmentally-responsive linker and releases a modified nucleic acid barcode from an in vivo sensor. Additional pH-responsive linkers include hydrazones and cis-Aconityl linkers. For example, hydrazones or cis-Aconityl linkers can be used to attach a modified nucleic acid barcode to the scaffold and the linker undergoes hydrolysis in an acidic environment.

Another non-limiting example of an environmentally-responsive linker is a temperature-sensitive linker that changes structure at a particular temperature (e.g., a temperature above or below 37 degrees Celsius). In some instances, a temperature above 37 degrees Celsius (e.g., as indicative of a fever associated with influenza) induces cleavage of an environmentally-responsive linker and releases a modified nucleic acid barcode from an in vivo sensor. In some embodiments, a temperature-sensitive linker is linked (e.g., tethered) to a scaffold.

In some embodiments, a temperature-sensitive linker undergoes a conformational change in response to a particular temperature. As a non-limiting example, a scaffold may be composed of one or more temperature-sensitive linkers encapsulating a modified nucleic acid barcode and in response to a particular temperature, the scaffold may become leaky and release the modified nucleic acid barcode. In one embodiment, a modified nucleic acid barcode is encapsulated (e.g., in a polymerosome, liposome, particle) by a temperature-sensitive linker, which is composed of NIPAM polymer. In some embodiments, the NIPAM polymer becomes leaky at one or more temperatures and releases an encapsulated modified nucleic acid barcode.

In some embodiments, a scaffold comprises one or more environmentally-responsive linkers (e.g., an environmentally-responsive linker that is responsive to pH, light, temperature, enzymes, light, or a combination thereof) and the scaffold encapsulates a modified nucleic acid barcode. In some instances, the scaffold encapsulating a modified nucleic acid barcode becomes degraded or leaky in response to a particular pH, temperature, presence of an enzyme, or light (e.g., a particular wavelength of light) and releases the modified nucleic acid barcode. In some embodiments, a scaffold encapsulating a modified nucleic acid barcode is a liposome, a polymersome, or a PLGA nanoparticle.

An environmentally-responsive linker (e.g., enzyme substrate, pH-sensitive linker, or a temperature-sensitive linker) may be attached directly to the scaffold. For instance it may be coated directly on the surface of the scaffold using known techniques. Alternatively if the scaffold is a protein material it may be directly connected through a peptide bond. Additionally, the environmentally-responsive linker may be connected to the scaffold through the use of another linker. Thus, in some embodiments the scaffold may be attached directly to the environmentally-responsive linker or indirectly through another linker. The other linker may simply be a spacer (or in other works be a linker that is not responsive to an environmental trigger). Another molecule can also be attached to a linker. In some embodiments, two molecules are linked using a transpeptidase, for example Sortase A.

In some embodiments, a linker comprises one or more cysteines. As a non-limiting example, a cysteine on a scaffold (e.g., an antibody) may be useful for conjugation of a nucleic acid barcode.

In some embodiments, a linker is not an environmentally-responsive linker that is cleaved in response to an environmental trigger. In some instances, a rigid linker may be used to prevent steric hindrance between two moieties. For example, a linker may comprise prolines. In some instances, a linker comprises the sequence SPSTPPTPSPSTPP (SEQ ID NO: 6). An environmentally-responsive linker may be linked to a scaffold through another linker that does not respond to the same environmental trigger. For example, a substrate for an enzyme may be linked to a scaffold through a linker that is not a substrate for the enzyme. Such a linker may be useful in preventing any interaction between the scaffold and the substrate that prevents substrate recognition and/or recognition of a targeting moiety on the scaffold. In some instances, a sensor comprises a scaffold with a protein (e.g., an antibody that targets the sensor to a particular cell type) and a linker that helps prevent the scaffold from interacting with an environmentally-responsive linker in the sensor. In some instances, a sensor comprises more than one environmentally-responsive linker and each environmentally-responsive linker may be connected to the scaffold through a rigid linker that prevents steric hindrance. For instance each sensor may include 1 type of environmentally-responsive linkers or it may include 2-1,000 different environmentally-responsive linkers or any integer therebetween. Alternatively each sensor may include greater than 1,000 environmentally-responsive linkers.

In some embodiments, a linker is a polymer such as PEG, a protein, a peptide, a polysaccharide, a nucleic acid, or a small molecule. In some embodiments the linker is a protein of 10-100 amino acids in length. Optionally, the linker may be 8 nm-100 nm, 6 nm-100 nm, 8 nm-80 nm, 10 nm-100 nm, 13 nm-100 nm, 15 nm-50 nm, or 10 nm-50 nm in length.

Examples of linking molecules include but are not limited to poly(ethylene glycol), peptide linkers, N-(2-Hydroxypropyl) methacrylamide linkers, elastin-like polymer linkers, and other polymeric linkages. Generally, a linking molecule is a polymer and may comprise between about 2 and 200 (e.g., any integer between 2 and 200, inclusive) molecules. In some embodiments, a linking molecule comprises one or more poly(ethylene glycol) (PEG) molecules. In some embodiments, a linking molecule comprises between 2 and 200 (e.g., any integer between 2 and 200, inclusive) PEG molecules. In some embodiments, a linking molecule comprises between 2 and 20 PEG molecules. In some embodiments, a linking molecule comprises between 5 and 15 PEG molecules. In some embodiments, a linking molecule comprises between 5 and 25 PEG molecules. In some embodiments, a linking molecule comprises between 10 and 40 PEG molecules. In some embodiments, a linking molecule comprises between 25 and 50 PEG molecules. In some embodiments, a linking molecule comprises between 100 and 200 PEG molecules.

In other embodiments, the second linker may be a second environmentally-responsive linker. The use of multiple environmentally-responsive linkers allows for a more complex interrogation of an environment. For instance, a first linker may be sensitive to a first environmental condition or trigger and upon exposure to an appropriate trigger undergoes a conformational change which exposes the second environmentally-responsive linker. When a second trigger is also present then the second environmentally-responsive linker may be engaged in order to release the modified nucleic acid barcode for detection. In this embodiment, only the presence of the two triggers in one environment would enable the detection of the modified nucleic acid barcode.

The sensitivity and specificity of an in vivo sensor may be improved by modulating presentation of the environmentally-responsive linker to its cognate environmental trigger, for example by varying the distance between the scaffold and the environmentally-responsive linker of the in vivo sensor. For example, in some embodiments, a polymer comprising one or more linking molecules is used to adjust the distance between a scaffold and an environmentally-responsive linker, thereby improving presentation of the environmentally-responsive linker to its cognate environmental trigger.

In some embodiments, the distance between a scaffold and an environmentally-responsive linker (e.g., enzyme substrate, pH-sensitive linker, or temperature-sensitive linker) ranges from about 1.5 angstroms to about 1000 angstroms. In some embodiments, the distance between a scaffold and an environmentally-responsive linker ranges from about 10 angstroms to about 500 angstroms (e.g., any integer between 10 and 500). In some embodiments, the distance between a scaffold and a substrate ranges from about 50 angstroms to about 800 angstroms (e.g., any integer between 50 and 800). In some embodiments, the distance between a scaffold and a substrate ranges from about 600 angstroms to about 1000 angstroms (e.g., any integer between 600 and 1000). In some embodiments, the distance between a scaffold and a substrate is greater than 1000 angstroms.

In some embodiments, a sensor described herein comprises a spacer, which may be useful in reducing steric hindrance of an environmental trigger from accessing an environmentally-responsive linker. In some embodiments, a spacer comprises at least 1, 2, 3, 4, 5, 6, 7, 8, 9, 10, 11, 12, 13, 14, 15, 16, 17, 18, 19, 20, 30, 40, 50, 60, 70, 80, or 90 amino acids (e.g., glycine). In some embodiments, a spacer is a polyethelyne glycol (PEG) spacer (e.g., a PEG spacer that is at least 100 Da, at least 200 Da, at least 300 Da, at least 400 Da, at least 500 Da, at least 600 Da, at least 700 Da, at least 800 Da, at least 900 Da, at least 1,000 Da, at least 2,000 Da, at least 3,000 Da, at least 4,000 Da, at least 5,000 Da, at least 6,000 Da, at least 7,000 Da, at least 8,000 Da, at least 9,0000 Da or at least 10,000 Da). In some embodiments, a PEG spacer is between 200 Da and 10,000 Da. In some embodiments, a spacer sequence is located between a scaffold and an environmentally-responsive linker. In some embodiments, a spacer sequence is located between the environmentally-responsive linker and the modified nucleic acid barcode.

In some embodiments, a linker separates two ligands. For example, a reporter may comprise two ligands that are connected through a linker. In some embodiments, a ligand is a detection ligand. In some embodiments, a ligand is a detection ligand. In some embodiments, a ligand is an antigen (e.g., an antigen that is recognized by an antibody). A capture ligand is a molecule that is capable of being captured by a binding partner. The detection ligand is a molecule that is capable of being detected by any of a variety of methods. While the capture ligand and the detection ligand will be distinct from one another in a particular detectable marker, the classes of molecules that make us capture and detection ligands overlap significantly. For instance, many molecules are capable of being captured and detected. In some instances these molecules may be detected by being captured or capturing a probe. The capture and detection ligand each independently may be one or more of the following: a protein, a peptide, a polysaccharide, a nucleic acid, a fluorescent molecule, or a small molecule, for example. In some embodiments the detection ligand or the capture ligand may be, but is not limited to, one of the following: Alexa488, TAMRA, DNP, fluorescein, Oregon Green, Texas Red, Dansyl, BODIPY, Alexa405, Cascade Blue, Lucifer Yellow, Nitrotyrosine, HA-tag, FLAG-tag, His-tag, Myc-tag, V5-tag, S-tag, biotin or streptavidin. See also, e.g., International Publication No. WO 2014/197840.

### Methods to Detect Environmental Triggers in Samples

Aspects of the disclosure relate to the surprising discovery that sensors comprising a modified nucleic acid barcode are useful for detecting an environmental trigger in vivo. As an example, a sensor of the present disclosure may be used to detect in vivo enzyme (e.g., protease) activity, a particular pH, light (e.g., at a particular wavelength), or temperature in a biological sample from a subject.

As used herein, a biological sample is a tissue sample (such as a blood sample, a hard tissue sample, a soft tissue sample, etc.), a urine sample, saliva sample, fecal sample, seminal fluid sample, cerebrospinal fluid sample, etc. In preferred embodiments, the biological sample is a tissue sample. The tissue sample may be obtained from any tissue of the subject, including brain, lymph node, breast, liver, pancreas, colon, liver, lung, blood, skin, ovary, prostate, kidney, or bladder. The tissue from which the biological sample is obtained may be healthy or diseased. In some embodiments, a tissue sample comprises tumor cells or a tumor. In some embodiments, a biological sample is not from a disease site. For example, a biological sample may be a urine sample from a subject with cancer.

A tissue sample for use in methods described by the disclosure may be unmodified (e.g., not treated with any fixative, preservative, cross-linking agent, etc.) or physically or chemically modified. Examples of fixatives include aldehydes (e.g., formaldehyde, formalin, glutaraldehyde, etc.), alcohols (e.g., ethanol, methanol, acetone, etc.), and oxidizing agents (e.g., osmium tetroxide, potassium dichromate, chromic acid, potassium permanganate, etc.). In some embodiments, a tissue sample is cryopreserved (e.g., frozen). In some embodiments, a tissue sample is embedded in paraffin.

A sensor of the present disclosure may also be used to detect an environmental trigger (e.g., enzyme, pH, light, or temperature) in vitro. As an example, an in vitro sensor may be added to a biological sample to assess enzyme activity.

### Methods for Detecting Disease in a Subject

In some aspects, the disclosure provides methods for detecting disease (e.g., cancer, pulmonary embolism, inflammation, and infectious diseases, such as, bacterial infections, viral infections (e.g., HIV) and malaria) in a subject. As used herein, a subject is a human, non-human primate, cow, horse, pig, sheep, goat, dog, cat, or rodent. In all embodiments human subjects are preferred. In aspects of the invention pertaining to disease diagnosis in general the subject preferably is a human suspected of having a disease, or a human having been previously diagnosed as having a disease. Methods for identifying subjects suspected of having a disease may include physical examination, subject's family medical history, subject's medical history, biopsy, or a number of imaging technologies such as ultrasonography, computed tomography, magnetic resonance imaging, magnetic resonance spectroscopy, or positron emission tomography.

In some embodiments, methods described by the disclosure result in identification (e.g., detection) of a disease in a subject prior to the onset of symptoms. In some embodiments, a tumor that is less than 1 cm, less than 0.5 cm, or less than 0.005 cm is detected using methods described by the disclosure. In some embodiments, the tumor that is detected is between 1 mm and 5 mm in diameter (e.g., about 1 mm, 2 mm, 3 mm, 4 mm, or about 5 mm) in diameter. In some embodiments, a pathogen-specific enzyme (e.g., a pathogen-specific protease) is detected (e.g., in a sample from a subject administered a sensor) during the incubation period of an infectious disease. In some embodiments, a subject with an infectious disease is contagious.

In some embodiments, the presence of an environmental trigger indicative of a disease (e.g., enzyme, pH, light, or temperature) in a subject is identified by obtaining a biological sample from a subject that has been administered a sensor as described by the disclosure and detecting the presence of a modified nucleic acid barcode in the biological sample. Generally, the biological sample may be a tissue sample (such as a blood sample, a hard tissue sample, a soft tissue sample, etc.), a urine sample, saliva sample, fecal sample, seminal fluid sample, cerebrospinal fluid sample, etc.

Detection of one or more modified nucleic acid barcodes in the biological sample may be indicative of a subject having a disease (e.g., cancer, pulmonary embolism, liver fibrosis, inflammation, and infectious diseases, including, bacterial infections, viral infections (e.g., HIV) and malaria). In some instances, detection of one or more detectable markers in the biological sample is indicative of a specific stage of a disease (e.g., metastatic or non-metastatic, contagious or non-contagious, etc.). In some embodiments, detection of one or more modified nucleic acid barcodes in the biological sample is indicative of a type of disease (e.g., type of cancer, type of bacterial infection, type of viral infection, or disease of a particular tissue). In some embodiments, an activity profile is determined for a subject responsive to detection of one or more detectable markers in the biological sample. As used herein, an activity profile refers to a value for the presence or absence of a plurality of enzymatic activities in a subject. In some embodiments, an activity profile is the aggregate information available when the presence and/or absence of a plurality of enzymatic activities is determined for a sample or subject. For example, a sample (e.g., a urine sample) from a subject may comprise two different modified nucleic acid barcodes indicative of the presence of two different enzymatic activities in the subject. The same sample may lack a third modified nucleic acid barcode, indicative of the absence of a detectable level of a third enzymatic activity in the subject. The presence of the first two enzymatic activities and the absence of a detectable level of the third enzymatic activity may comprise an exemplary activity profile for the subject. In some embodiments, an activity profile is used to diagnose a subject as having a disease, a specific stage of a disease, or a type of a disease, e.g., based upon the association of said disease with one or more enzymatic activities (or lack of one or more enzymatic activities) as described herein.

Any of the Cas-based nucleic acid detection systems described herein may be used to detect a modified nucleic acid.

### Administration

Compositions comprising any of the in vivo sensors described herein can be administered to any suitable subject. In some embodiments, the in vivo sensors of the disclosure are administered to the subject in an effective amount for detecting an environmental trigger (e.g., enzyme activity, pH, light, or temperature). An “effective amount”, for instance, is an amount necessary or sufficient to cause release of a modified nucleic acid barcode in the presence of an environmental trigger (e.g., enzyme activity, pH, light, or temperature). The effective amount of an in vivo sensor of the present disclosure described herein may vary depending upon the specific compound used, the mode of delivery of the compound, and whether it is used alone or in combination. The effective amount for any particular application can also vary depending on such factors as the disease being assessed or treated, the particular compound being administered, the size of the subject, or the severity of the disease or condition as well as the detection method. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective regimen can be planned.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated. The agent may comprise different types of carriers depending on whether it is to be administered in solid, liquid or aerosol form, and whether it need to be sterile for such routes of administration as injection.

In some embodiments, a dosage of less than 10 mg/kg of a sensor disclosed herein is administered to a patient (e.g., between 0.05 and 0.5 mg/kg, between 0.1 and 1 mg/kg, between 0.1 mg/kg and 1 mg/kg, between 5 mg/kg and 10 mg/kg, between 0.05 and 10 mg/kg, between 0.1 mg/kg and 0.3 mg/kg, or between 0.05 mg/kg and 0.3 mg/kg). In some instances, less than 0.3 mg/kg of a sensor is administered to a subject.

Aspects of the disclosure relate to systemic administration of an in vivo sensor to a subject. In some embodiments, the systemic administration is injection, optionally subcutaneous injection. The in vivo sensors of the present disclosure may also be administered through any suitable routes. For instance, the compounds of the present invention can be administered intravenously, intradermally, intratracheally, intraarterially, intralesionally, intratumorally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference). In some instances, a sensor is administered through a wearable device. In some instances, administration of a sensor disclosed herein does not require a phlebotomist and allows for patient self-monitoring of disease progression.

Multiple copies of the sensor are administered to the subject. Some mixtures of sensors may include enzyme susceptible detectable markers that are enzymes, others may be enzymatic susceptible domains, and other may be mixtures of the two. Additionally a plurality of different sensors may be administered to the subject to determine whether multiple enzymes and/or substrates are present. In that instance, the plurality of different sensors includes a plurality of detectable markers, such that each enzyme susceptible domain is associated with a particular detectable marker or molecules.

### EXAMPLES

### Example 1. Multiplexed In Vivo Disease Sensing with Nucleic Acid-Barcoded Reporters Allows for CRISPR-Cas-Based Detection

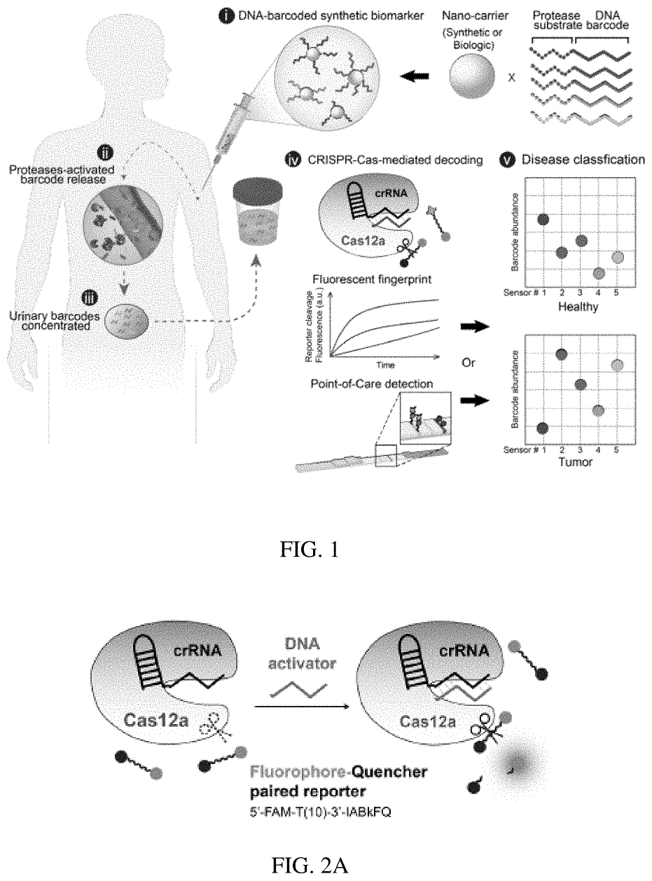

A system was developed to increase the number of protease-activated nanosensors that were testable in vivo. The in vivo sensors were barcoded with chemically-stabilized DNA. These barcodes were read in CRISPR-Cas12a-based enzymatic assays (FIG. 1). Briefly, Cas12a enzymes (ENGEN® Lba Cas12a was utilized in this study) assembled with guide CRISPR RNA sequences (crRNAs) recognize 1) a T nucleotide-rich protospacer-adjacent motif (PAM) to target dsDNA for gene-editing applications; 2) ssDNA through sequence complementarity in a PAM independent manner, unleashes robust, nonspecific ssDNA trans-cleavage activity that can be monitored using a fluorophore (F)-quencher (Q)-labeled reporter (poly(T)). It was demonstrated for the first time that, in addition to native ssDNA, LbaCas12a can be activated by fully chemically-modified (phosphorothioate) ssDNA (FIG. 2B). When injected into a small animal model (i.e. Balb/c mouse), native ssDNA collected in the urine couldn't activate LbaCas12a assembled with corresponding crRNA, due to the unspecific DNase activities in the serum. In contrast, different lengths of phosphorothioate-modified ssDNAs in solution or unprocessed urine after intravenous injection triggered the trans-cleavage activity of LbaCas12a (FIG. 2D). Notably, the 20-mer crRNA-complementary ssDNA optimized kidney filtration into urine and reporter cleavage activity. Furthermore, multiple crRNA-modified ssDNA activator pairs were validated with orthogonality between different sequences allowing for parallel readout in multiple well assays (FIG. 2E). The CRISPR nuclease can be activated once it sees its programmed DNA target in unprocessed urine and cleaves a tagged construct that rapidly appears on a lateral-flow paper strip. The cleaved reporter was detected as shown in FIG. 2F. This detection step can happen within 1 hr at the point of care.

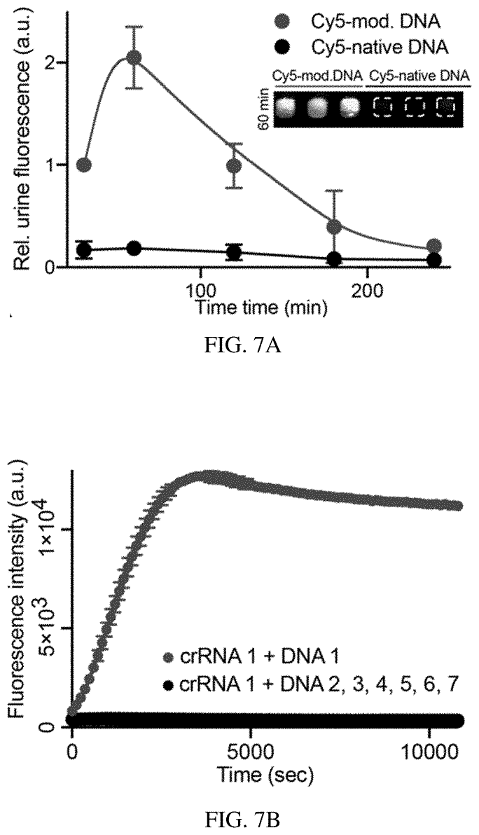

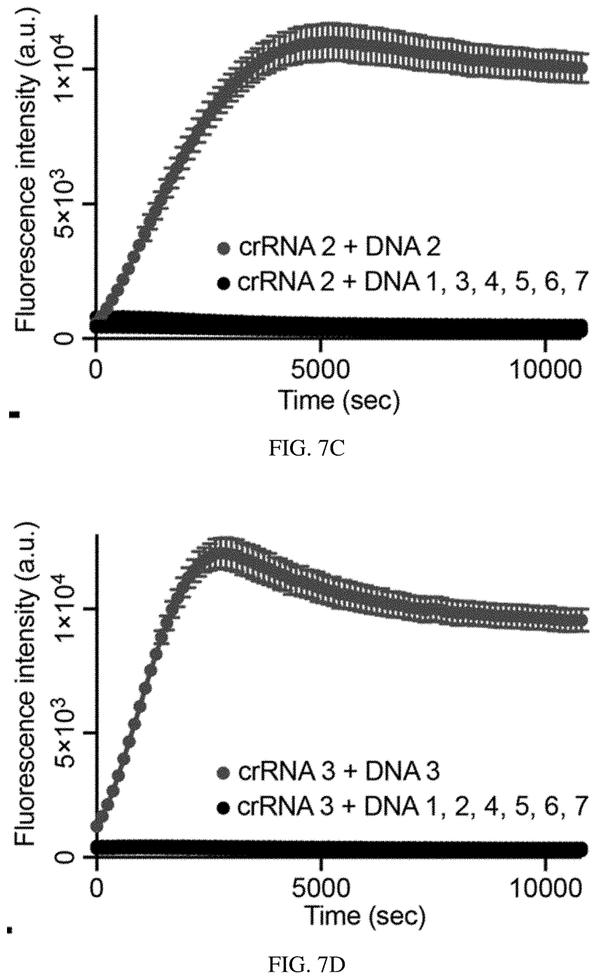

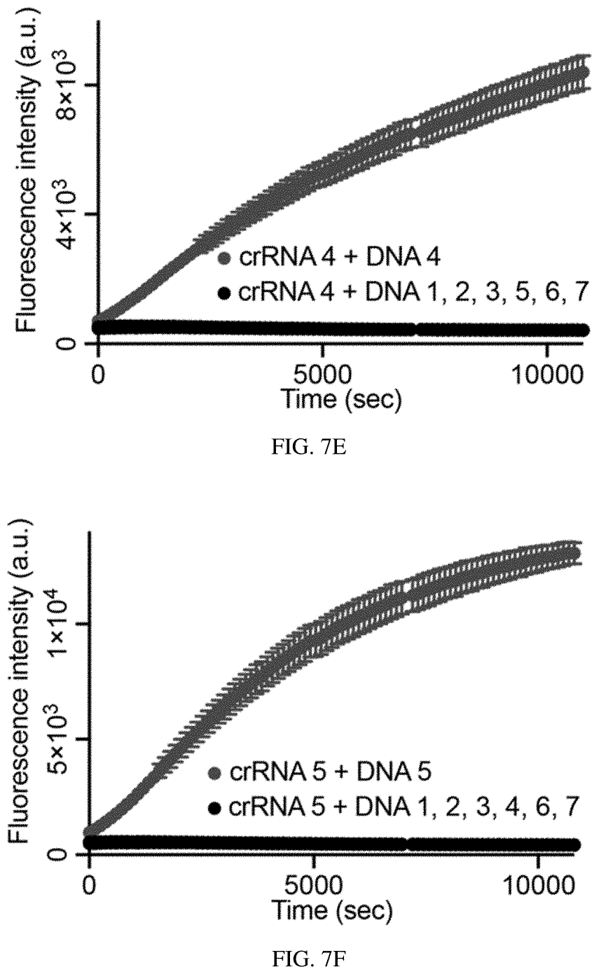

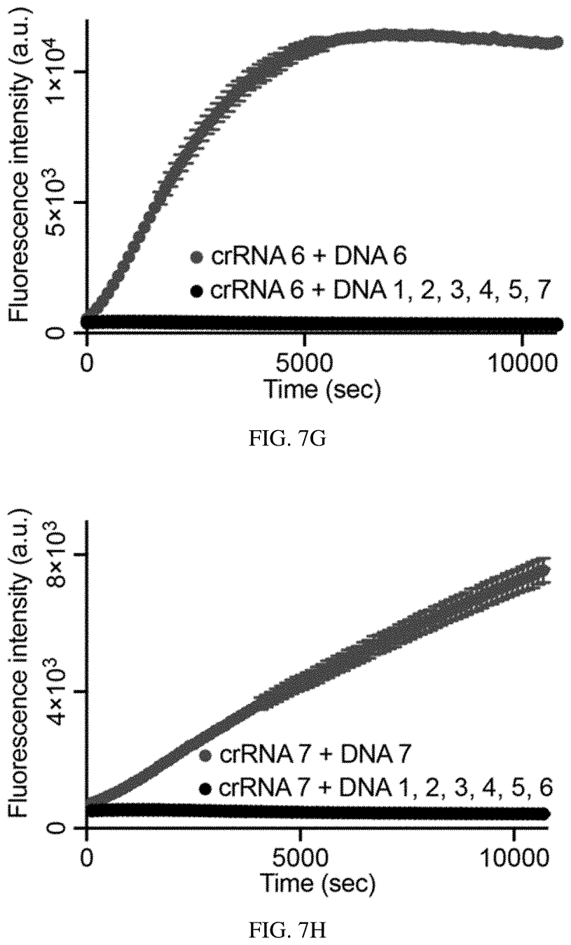

In particular, the Cas12a from Lachnospiraceae bacterium ND2006 (LbaCas12a, UniProtKB Accession No. A0A182DWE3) assembled with guide CRISPR RNA sequences (crRNAs) recognizes 1) a T nucleotide-rich protospacer-adjacent motif (PAM) to target double-stranded DNA (dsDNA), or 2) single-stranded DNA (ssDNA) through sequence complementarity in a PAM-independent manner, and unleashes a robust, nonspecific ssDNA trans-cleavage activity that can be monitored using a fluorophore (F)-quencher (Q)-labeled reporter (FIG. 2A) (Chen et al. Science 360, 436-439 (2018)). In addition to native dsDNA or ssDNA, LbaCas12a was activated by phosphorothioated ssDNA at a relatively slower speed (FIG. 2B). When intravenously administered into a murine model (Balb/c mouse), native ssDNA in urine collected from injected animals could not activate LbaCas12a assembled with the corresponding crRNA due to the unspecific DNase activities in circulation (FIGS. 2C-2D, FIG. 7A). In contrast, different lengths of phosphorothioate-modified ssDNAs in solution or unprocessed urine from injected animals triggered the trans-cleavage activity of LbaCas12a (FIG. 2D). Notably, the 20-mer crRNA-complementary ssDNA optimized kidney filtration into urine, producing the highest reporter cleavage activity, whereas the 24-mer ssDNA containing the PAM sequence produced the highest cleavage signal in vitro (FIG. 2D, Tables 5-6). In Table 6, activation of Cas12a with native and modified DNA oligos was quantified in the Cas12a fluorescent cleavage assay. For ‘DNA in vitro’, 4 nM of DNA activators with different length were added in each reaction. For ‘DNA in vivo’, 1 nmol of DNA activators, native or modified, with different length were injected into healthy Balb/c mice and urine samples collected after 1 h of injection were added in each reaction.

Furthermore, multiple crRNA-modified ssDNA activator pairs were validated with orthogonality between different sequences, allowing for parallel readout in multiple well assays (FIG. 2E, FIGS. 7B-7H). The LbaCas12a was activated once it encountered its programmed DNA target in unprocessed urine and cleaved a FAM and biotin dual-tagged ssDNA reporter that rapidly appeared on a lateral flow paper strip (FIG. 2F). The presence of the ‘sample band’ at the front of the strip indicated that the cleaved reporters were produced upon the activation of LbaCas12a by the urinary DNA activator.

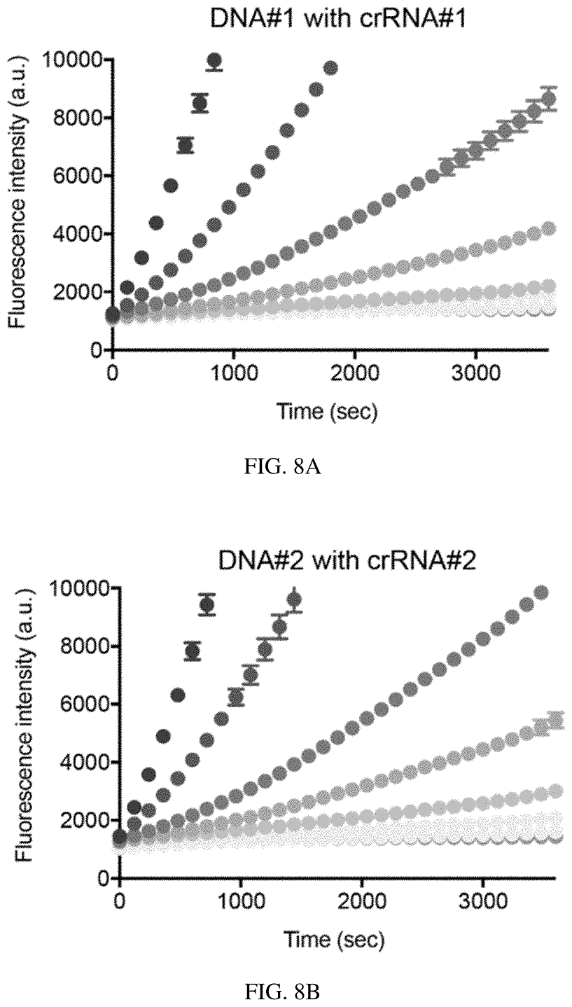

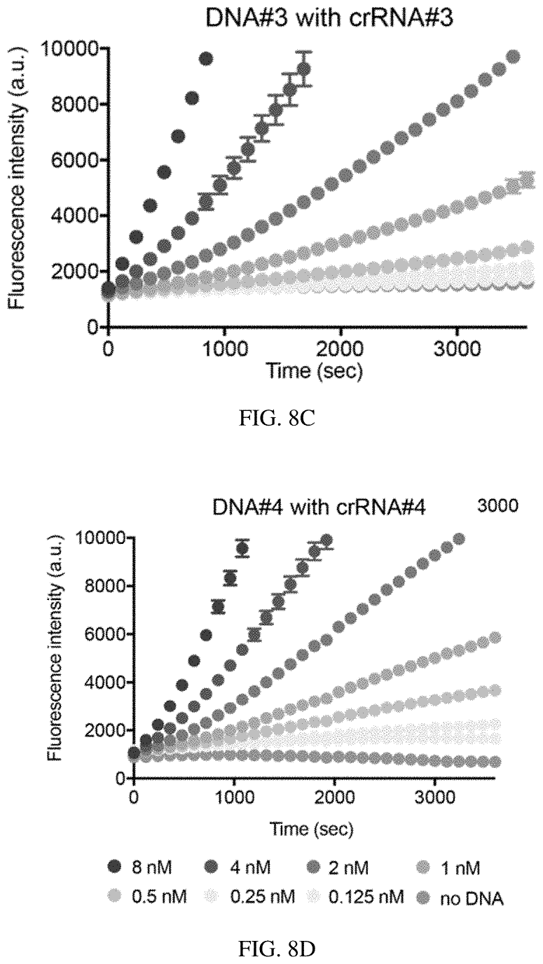

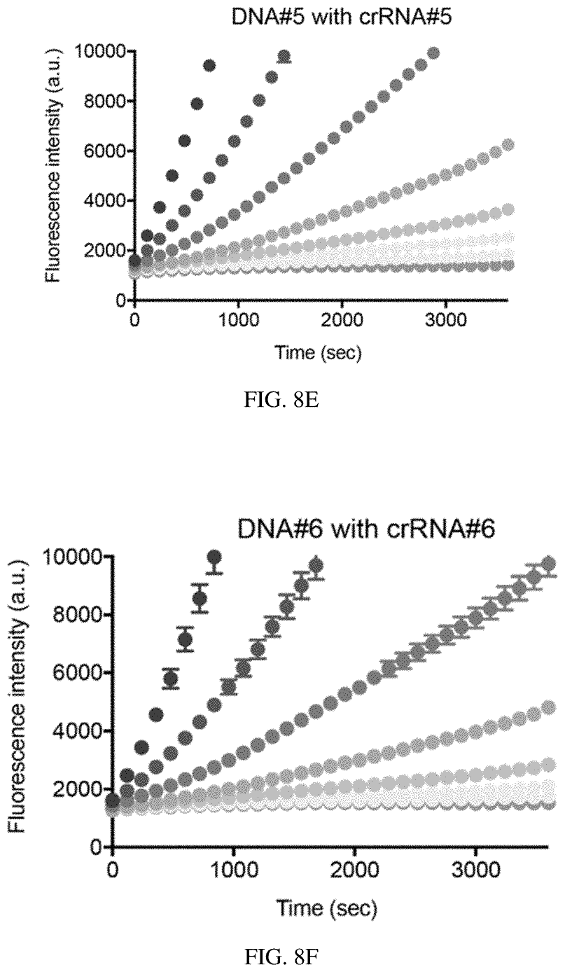

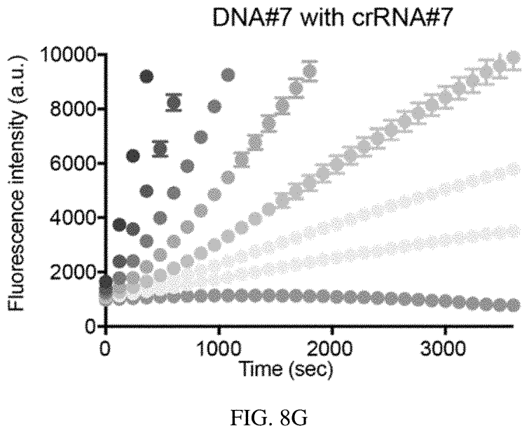

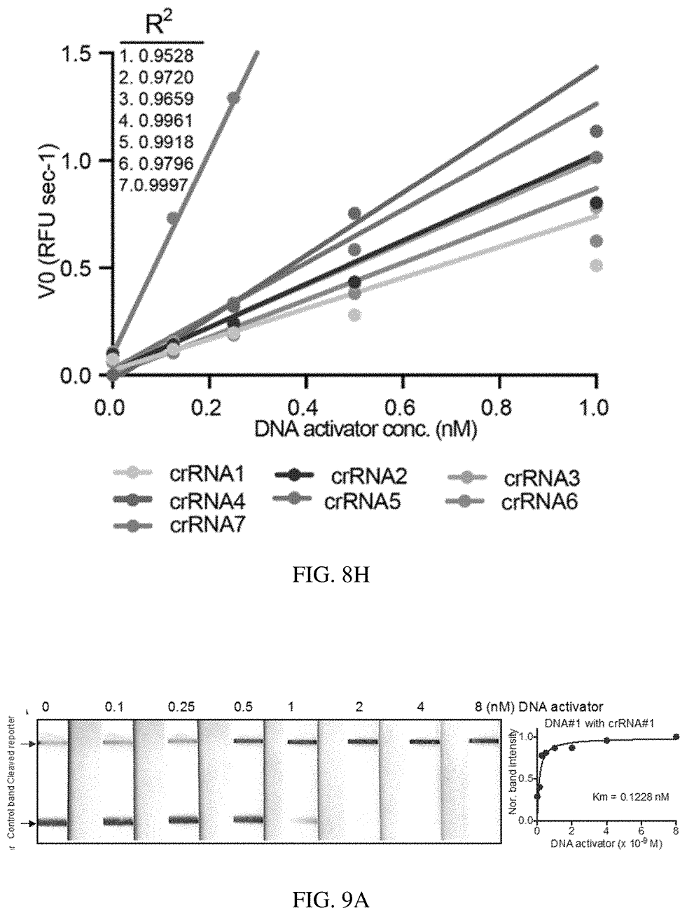

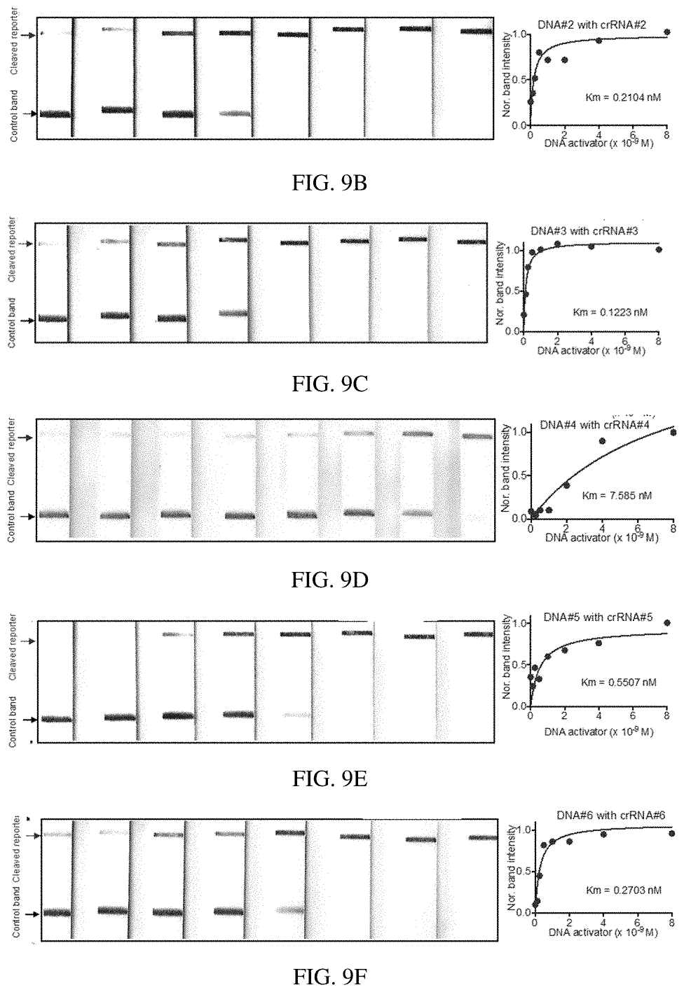

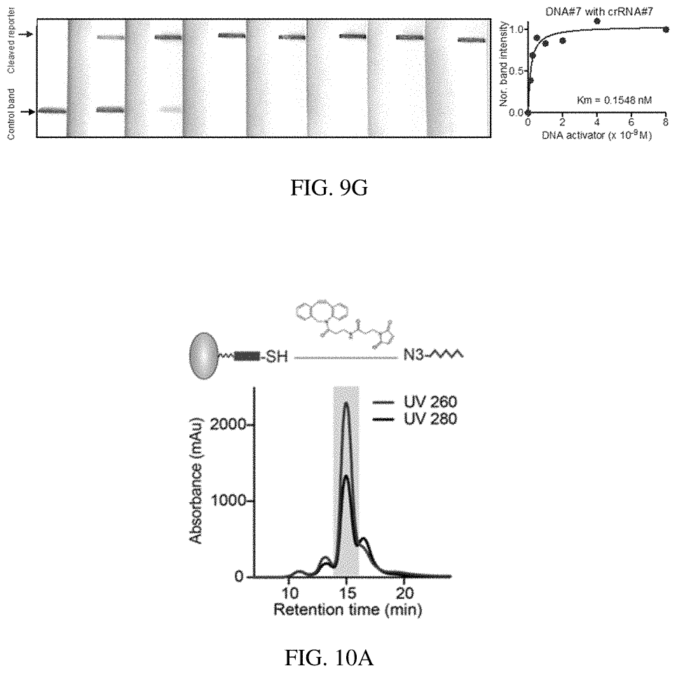

In addition to the binary DNA activator detection, quantification of the intensity of the sample bands on paper strips allowed assessment of enzymatic kinetics (FIG. 2G). By adjusting the concentrations of assay components, the working linear ranges fell within sub-nanomolar DNA activator concentrations for both fluorescent and paper-based readouts (FIGS. 8A-8H and FIGS. 9A-G).

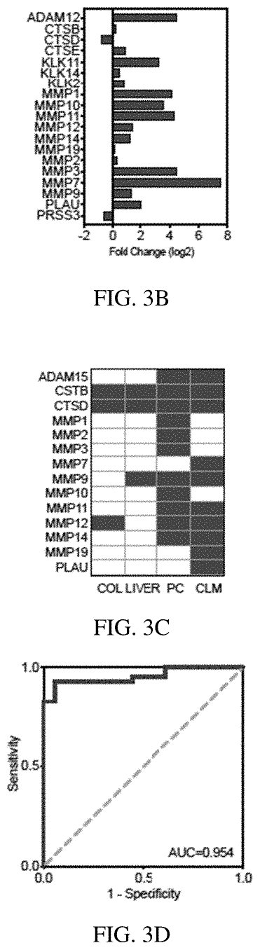

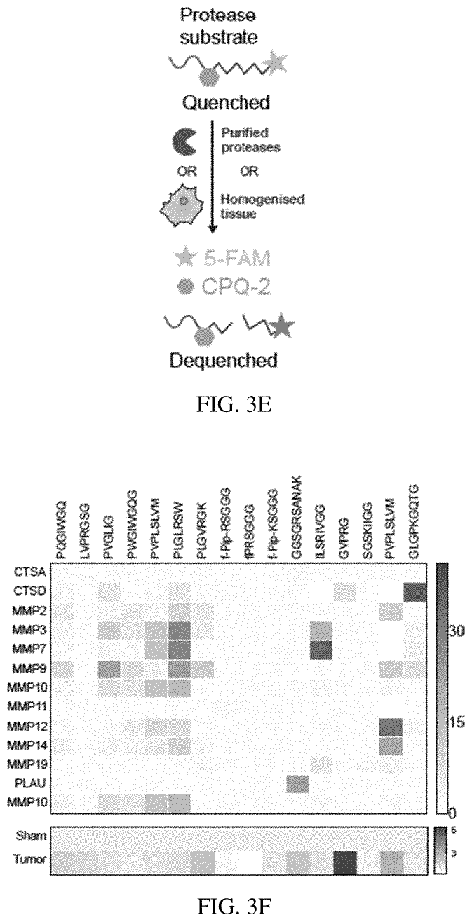

To develop efficient tools for precision diagnosis, the protease-dependent environment of disease settings was first leveraged to cleave and release the phosphorothioate modified DNA barcodes that are size-specifically concentrated in the urine, thus resulting in a non-invasive readout for the presence of the target disease (FIG. 3A). These DNA-barcoded activity-based nanosensors (ABNs) contain peptide substrates subject to cleavage of disease-associated proteases. Using a colorectal cancer (CRC) metastasis model, aberrant proteases and specific substrates were first identified for invasive CRC classification. A panel of CRC proteases was identified through transcriptomic and proteomic analysis. Transcriptomic data in The Cancer Genome Atlas (TCGA) was queried to identify proteases overexpressed in 476 colon adenocarcinoma samples versus 41 normal adjacent tissue samples (FIG. 3B). Out of over 150 secreted and membrane-bound endoproteases in this dataset, multiple proteases expressed in tumors at levels >1.5-fold over NAT were identified among the well-studied matrix metalloproteinases (MMPs), serine and aspartic protease families (i.e. cathepsin, kallikrein-related peptidases). In addition to the transcriptome analysis, a proteomic strategy was developed to characterize the composition of extracellular matrix in normal tissues and tumors by enriching protein extracts for ECM components and mass spectrometry analysis (Naba et al., Molecular & cellular proteomics: MCP 2012, 11, M111 014647). Application of this method to profile patient specimens collected from distinct sources (normal liver and colon tissues, colon tumors, and CRC liver metastases) identified proteases specific for colon primaries and distant metastases (e.g. MMP-1, -9, -12, Cathepsin B, D) (matrisomeproject.mit.edu, FIG. 3C). Such information provided valuable references for development of the urinary readouts against metastatic specific proteases. In in vitro studies, a fluorogenic activity assay was developed to identify peptide substrates specific for target proteases. Cleavage kinetics of a given peptide substrate could be recorded by the increase of fluorescence upon cleavage of the flanking fluorescence resonance energy transfer (FRET) pair (FIG. 3E). To profile multiple protease-substrate interactions simultaneously, 16 peptide substrates were screened against purified recombinant enzymes or CRC tumor/healthy tissue lysis, and identified 5 top substrate candidates (Q7: PLGVRGK (SEQ ID NO: 1), Q9: fPRSGGG (SEQ ID NO: 2), PQ2: GGSGRSANAK (SEQ ID NO: 3), PQ12: GVPRG (SEQ ID NO: 4), PQ19: PVPLSLVM (SEQ ID NO: 5)) broadly covering metallo- and serine-protease activities to construct sensors for in vivo validation (FIG. 3F) (Dudani et al., Proceedings of the National Academy of Sciences of the United States of America 2018, 115, 8954; Kwong et al., Nature biotechnology 2013, 31, 63).

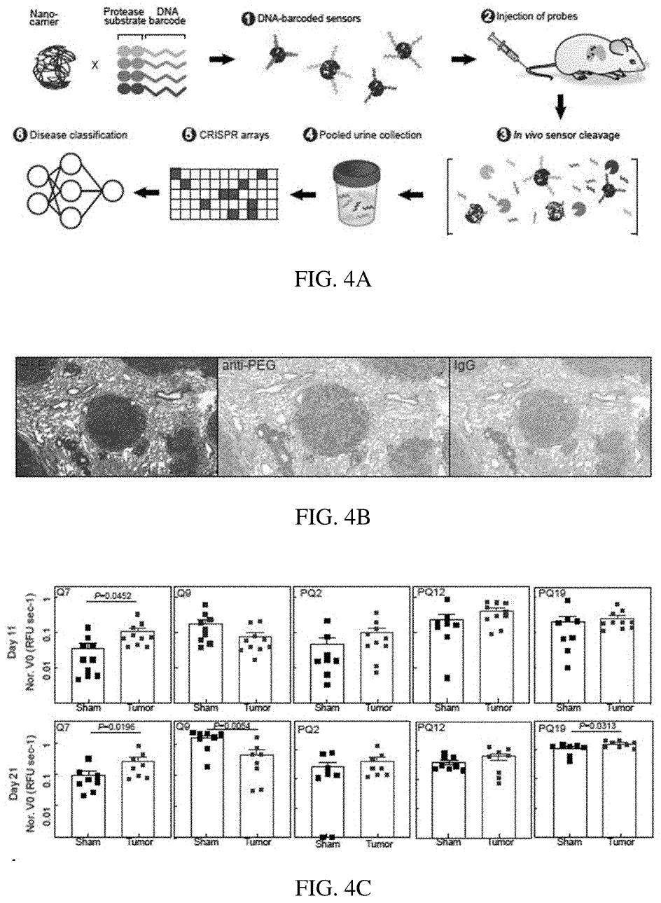

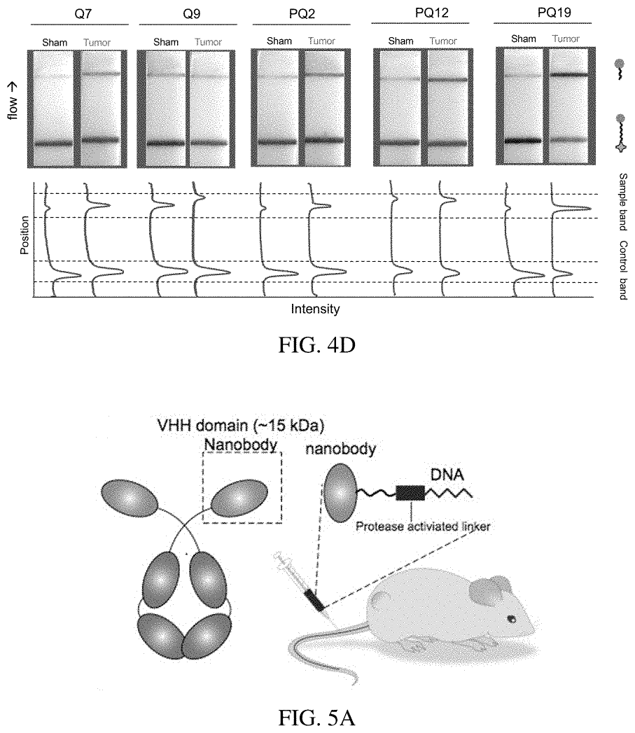

To improve the throughput of in vivo studies for enhanced detection specificity, fully modified oligonucleotides were used to barcode the sensors and administered them as a single pool to mice (FIG. 4A). To track the tumor accumulation patterns of the DNA-barcoded nanosensors, a CRC lung metastasis model was established via intravenous injection of CRC cells (MC26 cell line) in female Balb/c mice (FIG. 4A). It was first demonstrated that a 20-mer DNA-barcoded MMP-responsive ABN (DNA-Q7-ABN) constructed on a synthetic (8-arm polyethylene glycol) core accumulated in the CRC lung metastases following intravenous injection (FIG. 4B). Then the entire 5-plex of DNA-barcoded ABNs was tested in vivo, with an emphasis on identifying reporters to differentiate mice bearing lung metastases from the healthy controls. The multiplexed DNA-barcoded ABNs were intravenously administered to tumor-bearing mice over the course of metastasis development, and quantified urinary DNA barcodes that were freed from the nanosensors at 1 hr after injection. Urine samples were an analyzed by multiple-well LbaCas12a trans-cleavage assays by tracking the kinetics of cleavage upon fluorescence-quencher labeled poly(T) reporter. There were a few reporters that differentiated diseased mice from the healthy control group, with some reporter differences becoming amplified over time (Q7, Q9, Q19). These reporters corresponded to peptides cleaved by metallo-proteases in vitro and showed distinct cleavage patterns in tissue lysates from tumor vs healthy controls. As tumors invade (day 11 vs day 21 after tumor inoculation), an increase in the differences in urine signal from diseased and control mice was observed (FIG. 4C).

Multiplexed quantitative urinary DNA barcode detection was combined with lateral flow for visual readout to enable point-of-care diagnostics. In addition to the aforementioned Cas12a kinetic cleavage assays (FIG. 4C), the lateral flow assay was designed to detect biotin- and FAM-labelled amplicons. After the activation of LbaCas12a by incubating the enzyme with a specific crRNA and its complementary urinary DNA barcodes for 30 min, the enzyme complex and FAM-poly(T)-biotin labeled reporter were mixed and added onto an assigned location in 96-well plate. A series of lateral flow strips were loaded onto the plates and the multiple-pot test paper results appeared in 5 min at room temperature, enabling a high-throughput protocol. The bands on the strips were quantified and interpreted through comparing the fingerprints on test papers (FIG. 4D).

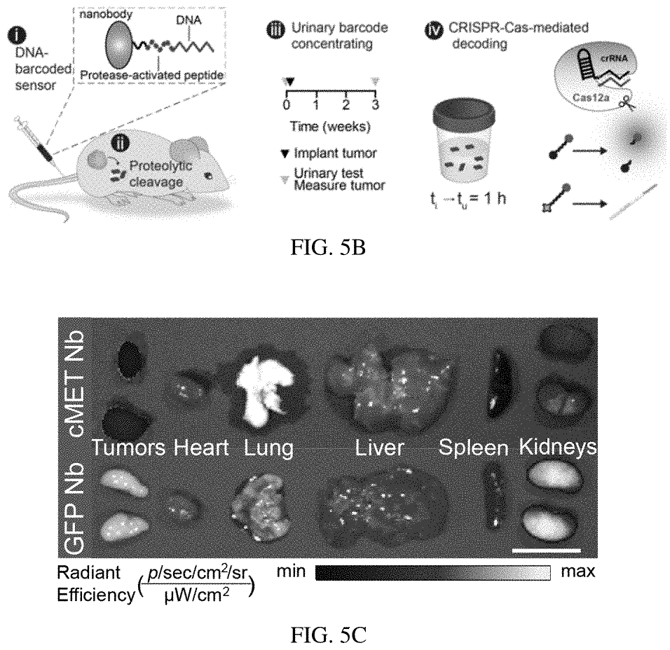

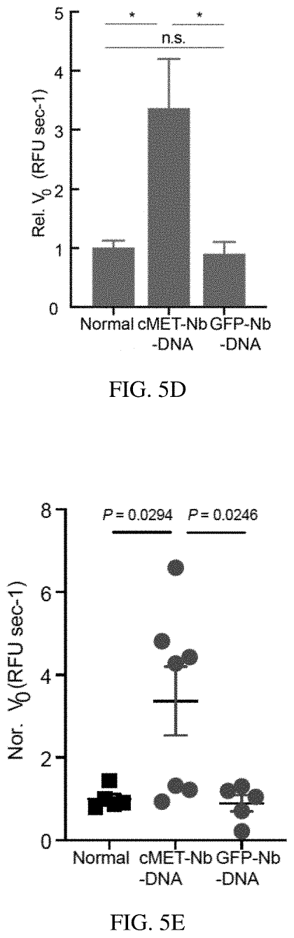

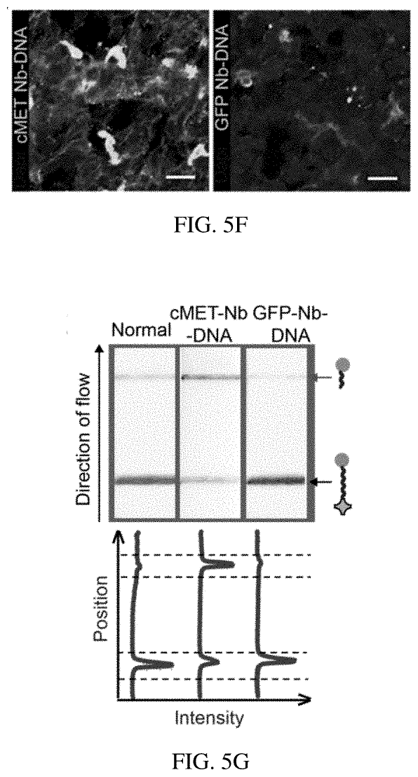

One major challenge for diagnosis and therapy of cancer is tailoring multiple disease signatures, which are defined by biological differences spanning genetic, transcriptomic, and proteomic differences between tumor and healthy tissue, while minimizing off-target effects (Hunter, K. Nature reviews. Cancer 2006, 6, 141). To this end, a tumor-targeting nanobody was re-engineered to construct protease-activatable nanobodies through programmable genetic encoding (FIGS. 5A-5B). Protease-activatable nanobodies were constructed by inserting a well characterized PLAU substrate (PQ2: GGSGRSANAK (SEQ ID NO: 3)) with an unpaired cysteine for one-step site-specific labeling of cargos via a thio-ether bond (see, e.g., Masa et al., Bioconjugate chemistry 2014, 25, 979). The cysteine was introduced at the carboxyl terminus, positioning the conjugation-site on the opposite side of the antigen-binding region to avoid antigen binding interference. To prevent possible misfolding caused by the internal disulfide bond present in the Nb, the peptide substrate with cysteine is spaced by a rigid linker (SPSTPPTPSPSTPP (SEQ ID NO: 6)) from the Nb sequence. The recombinant Nb with protease activated site and an unpaired cysteine (Nb-PAS) were purified from the periplasmic extract using affinity chromatography and subsequent size-exclusion chromatography and efficiently yielded comparable solubility with the original Nb (FIGS. 5A-5B).

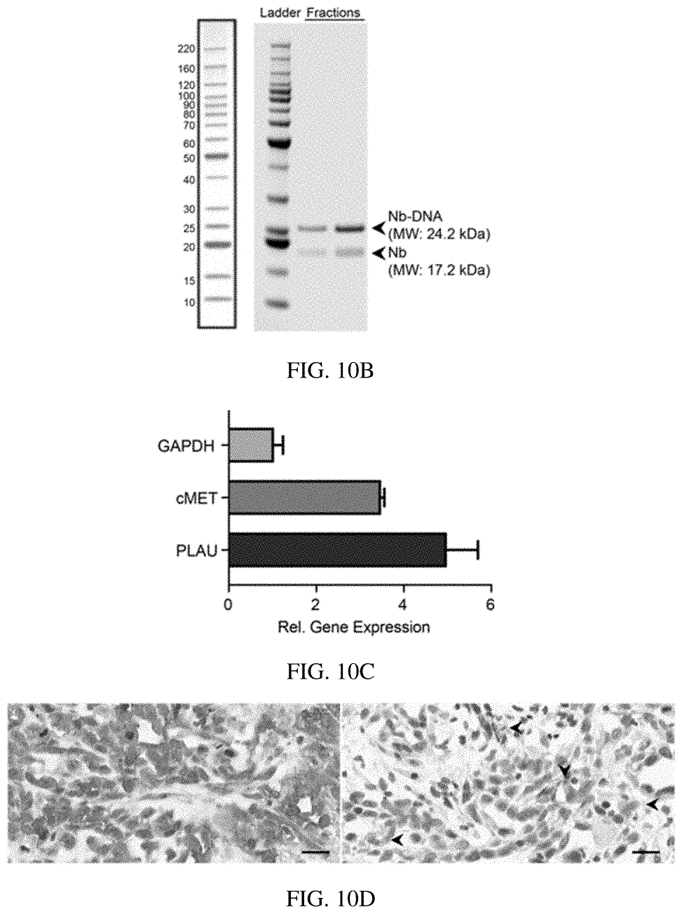

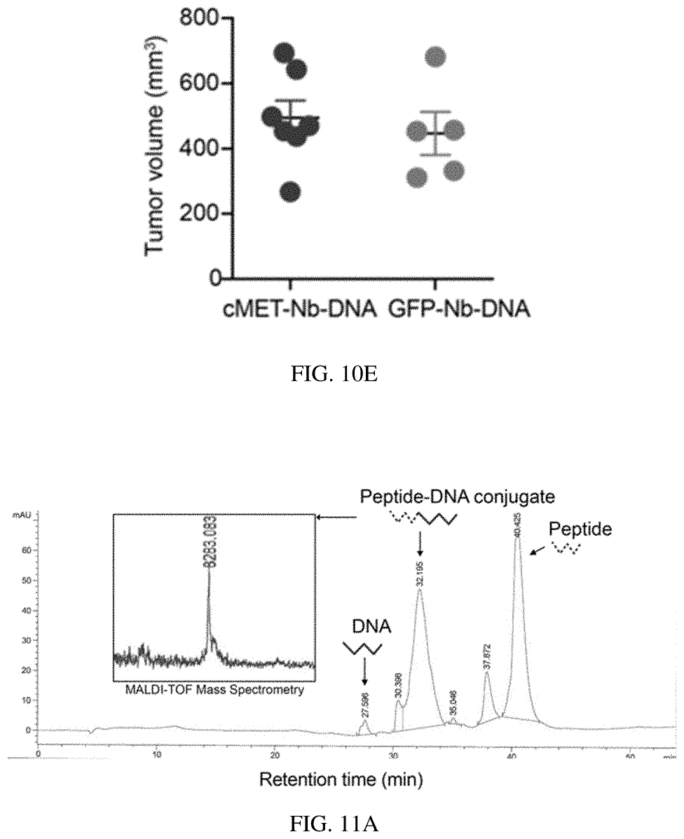

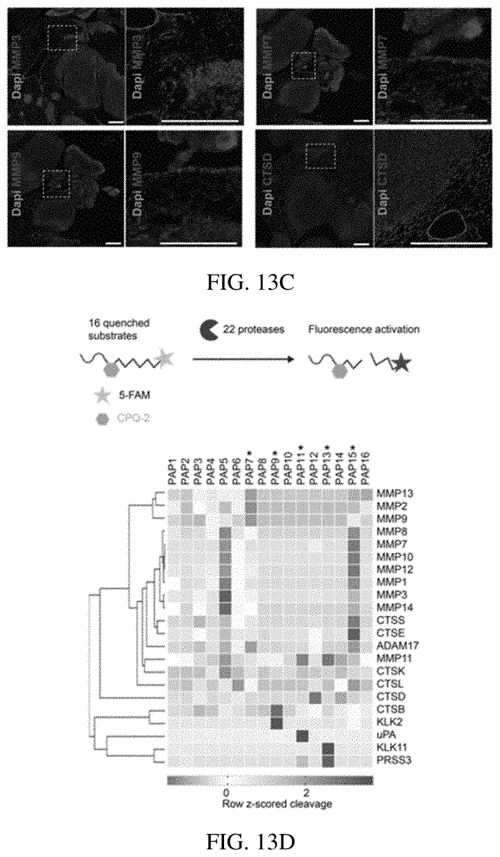

To develop DNA-encoded synthetic biomarkers, deregulated proteolytic activities in the disease microenvironment were leveraged to cleave and release the phosphorothioated DNA barcodes that were size-specifically concentrated in the urine to produce a noninvasive readout of the target disease. First, a singleplex synthetic biomarker was evaluated in vivo in a human prostate cancer (PCa) xenograft model29. To maximize the on-target protease cleavage, the DNA-SUB was engineered on a biological scaffold that enables tumor-targeting abilities. To utilize the robust stability and tissue affinity of single domain antibody fragments (nanobodies), DNA-encoded, protease-activatable nanobodies were instructed by inserting a peptide substrate sequence with an unpaired cysteine for one-step site-specific labeling of cargos via a thio-ether bond (FIG. 5B, Table 7, FIGS. 10A-10B) (Morrison, Nature reviews. Drug discovery 18, 485-487 (2019); Massa et al., Bioconjugate chemistry 25, 979-988 (2014); Kirley et al., Biochemical and biophysical research communications 480, 752-757 (2016); and Muyldermans, Annual review of biochemistry 82, 775-797 (2013)). The peptide substrate specifically responded to the PCa-associated protease PLAU29 (FIG. 13D). To prevent possible misfolding caused by the internal disulfide bond present in the nanobody, the peptide substrate with cysteine was spaced from the nanobody scaffold by a rigid linker (Table 7). For in vivo validation, a PLAU-activated, cMET-targeting nanobody was tested in the cMET- and PLAU-expressing PC-3 cell-derived tumor model. In the subcutaneous PC-3 tumors, the cMET nanobody mediated active tumor trafficking upon systemic administration (FIG. 5C and FIGS. 10C-10D), whereas the GFP nanobody did not. By the nanobody-mediated selective binding to the tumor upon systematic administration (FIG. 5C), the diagnostic signals triggered on-target through in vivo sensing of endogenous proteolytic activities in the tumor microenvironment, and release DNA barcodes detectable in the urine. In the PLAU-expressing PC-3 cell-derived human prostate cancer xenografts, administration of Nb-PAS-DNA resulted in significant increases in LbaCas12 trans-cleavage rate activated by urine samples collected from tumor-bearing mice, relative to that from the healthy controls in the fluorophore (F)-quencher (Q) labeled poly(T) reporter assay (FIG. 5D-5E) or the FAM-poly(T)-biotin reporter mediated, paper-based lateral flow assay (FIG. 5G).

PLAU-activated nanobodies covalently conjugated with the 20-mer DNA barcode were efficiently separated via size-exclusion chromatography. The DNA-barcoded, PLAU-activated cMET nanobody (cMET-Nb-DNA) exhibited enhanced tumor accumulation compared with the DNA-barcoded, PLAU-activated non-targeting GFP nanobody (GFP-Nb-DNA) (FIG. 5F) (Fridy et al. Nature methods 11, 1253-1260 (2014)). cMET-Nb-DNA was systemically administered to tumor-bearing and healthy control mice and quantified urinary DNA barcodes that were freed from the nanobody scaffold 1 h after injection. Urine samples were incubated with LbaCas12a-coupled with the complementary crRNA, and the trans-cleavage activity triggered by the DNA barcode was analyzed by tracking the kinetics of cleavage of a fluorescence-quencher labeled poly(T) reporter (FIG. 5B). Administration of cMET-Nb-DNA significantly increased the trans-cleavage rate of LbaCas12a activated by urine samples collected from tumor-bearing mice, relative to that of the healthy controls (FIG. 5D-5E). To translate the fluorescent readout into a PoC detection tool, LbaCas12a was activated activated by mouse urine samples with the FAM-poly(T)-biotin reporter and ran the cleavage products on lateral flow paper strips. An enhanced sample band appeared in samples collected from tumor-bearing mice injected with cMET-Nb-DNA (FIG. 5G). The high sensitivity and specificity of the sensor to track disease was reflected in a ROC curve (AUC=0.89) (FIG. 5H). In contrast, urine samples collected from tumor-bearing mice injected with GFP-Nb-DNA activated LbaCas12a at a rate that was almost identical to the samples from healthy controls, indicating that the release of the DNA barcodes was triggered on-target (FIGS. 5D and 5G).

### Example 2. DNA-Encoded Multiplex Synthetic Urine Biomarkers Longitudinally Monitor Disease Progression in a Portable Manner

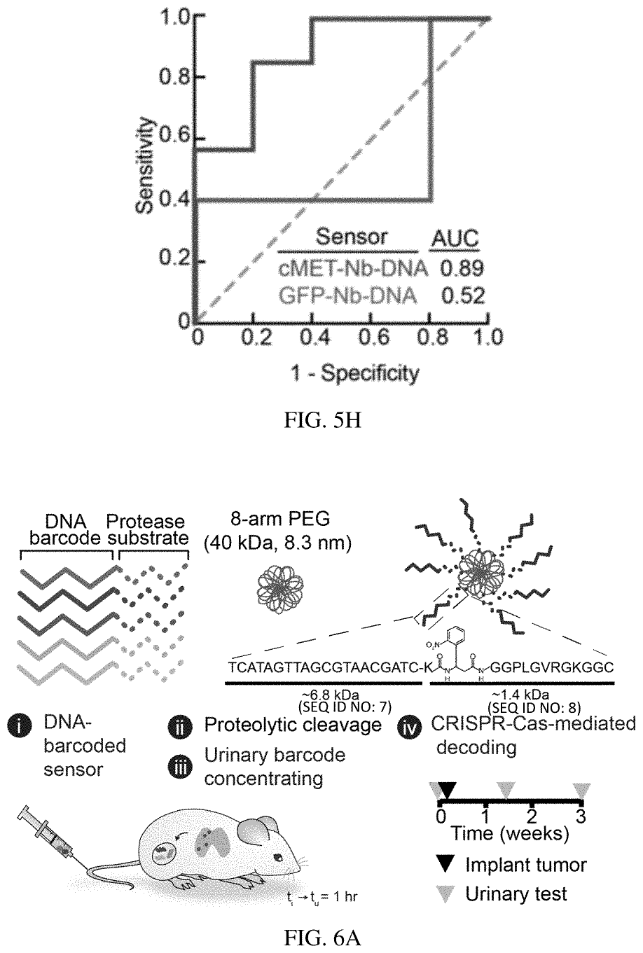

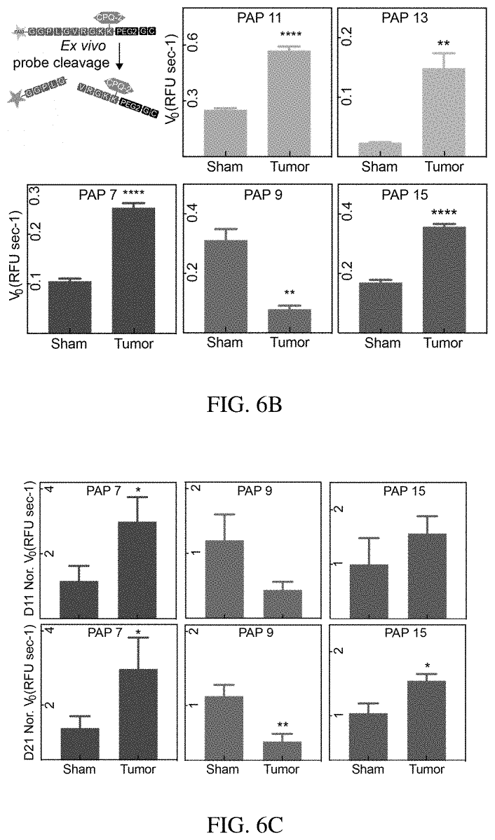

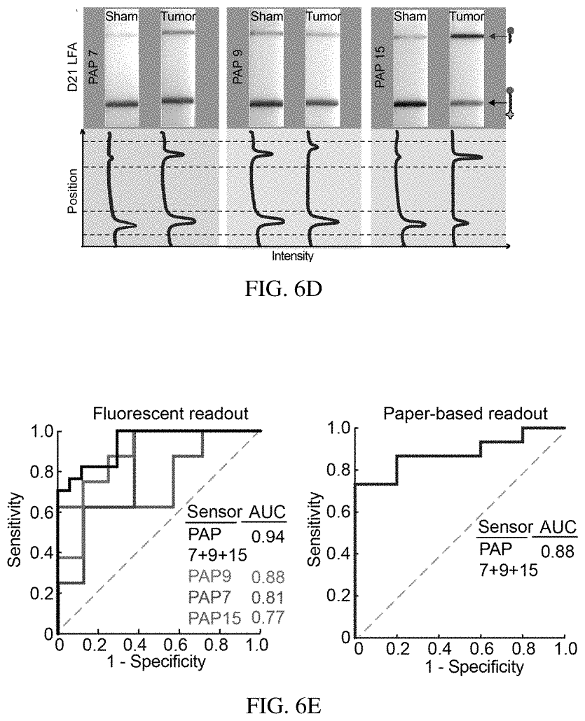

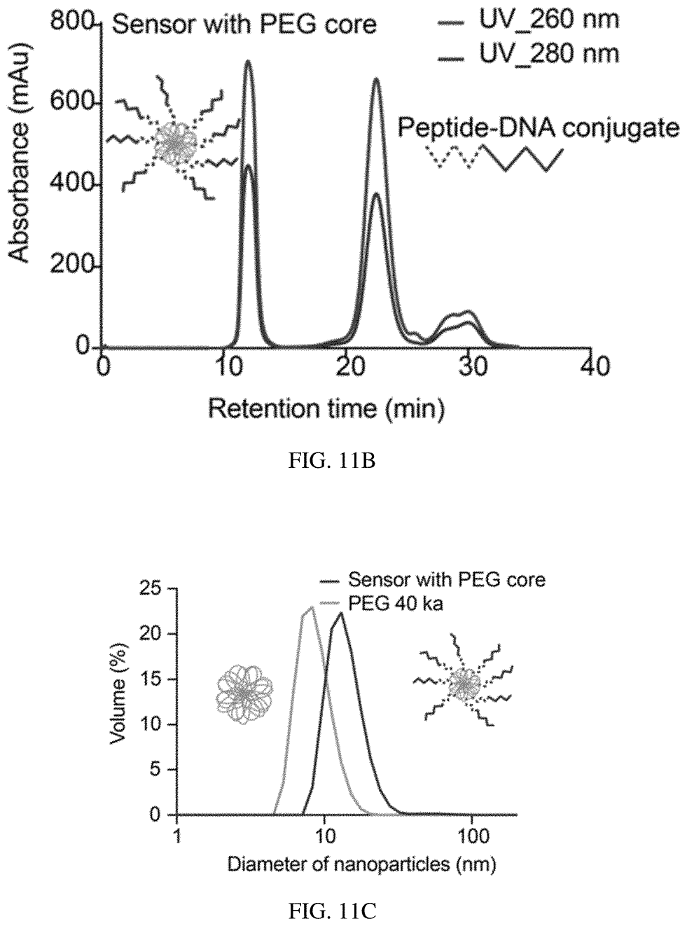

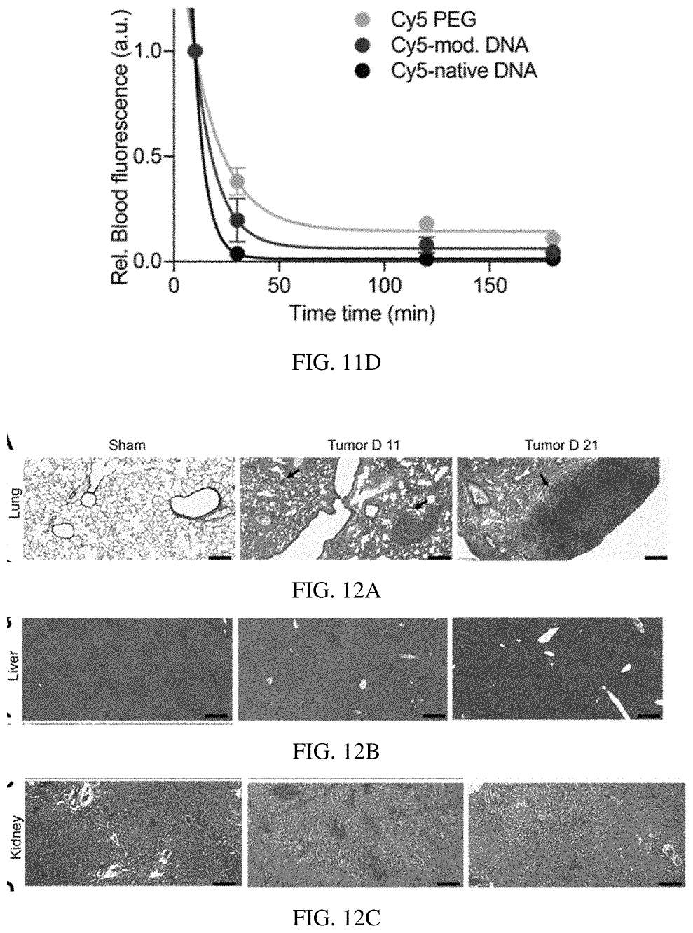

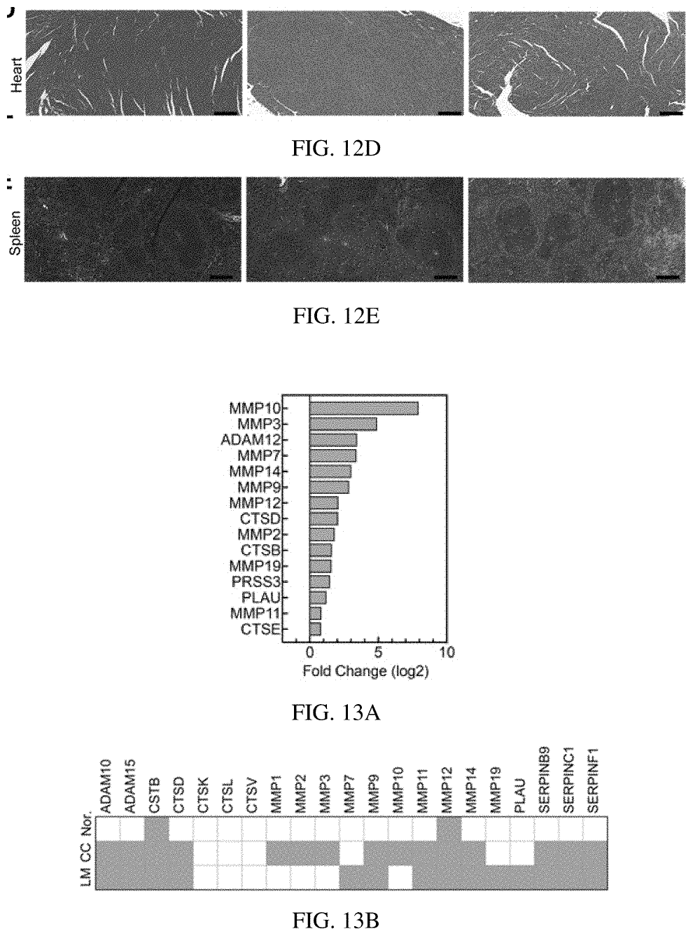

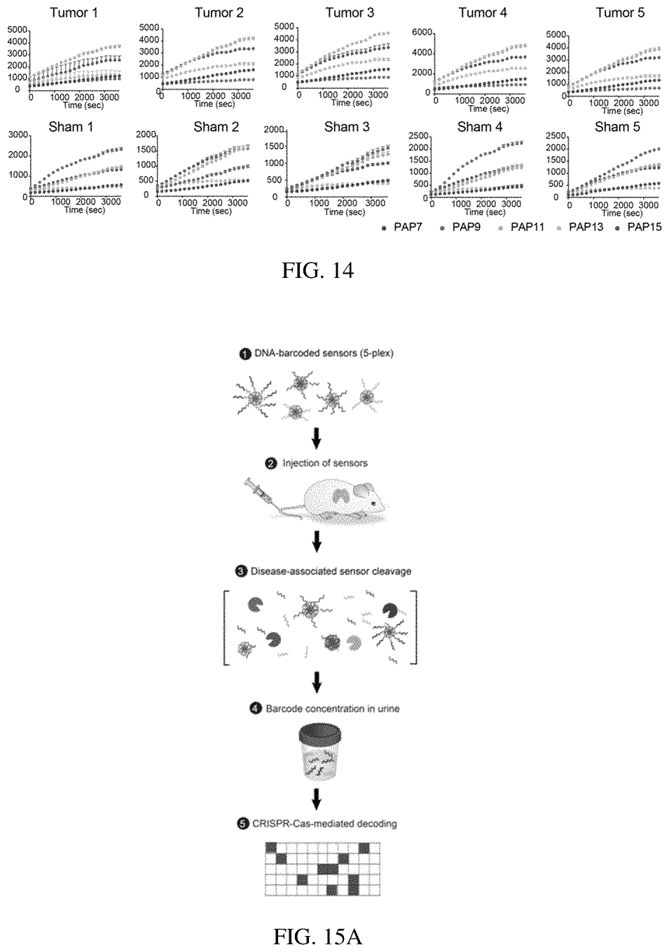

It is increasingly appreciated that analysis of multiple cancer hallmarks may optimize diagnostic sensitivity and specificity in heterogenous diseases. Whereas active targeting is limited to diseases that express specific ligands, multiplexing of an untargeted scaffold has the potential to be more generalizable. Therefore, a multiplexed panel of DNA-SUBs was constructed on a polymer-based scaffold and administered them as a single pool to mice (FIG. 6A). Each DNA-SUB was comprised of a 20-mer phosphorothioated DNA-tagged, protease-activated peptide (PAP) covalently conjugated to a synthetic polymer (8-arm polyethylene glycol, 40 kDa) (FIG. 6A, FIGS. 11A-11D & Table 7). To monitor nanosensor trafficking to tissue contact, a syngeneic mouse model was established by intravenously injecting a metastatic murine colorectal cell line (MC26-LucF) into immunocompetent Balb/c mice (FIG. 6A, FIGS. 12A-12E) (Danino et al. Science translational medicine 7, 289ra284 (2015). A panel of CRC-specific proteases was first identified through transcriptomic analysis and found multiple proteases expressed in tumors at >1.5-fold levels over normal samples, including members of the matrix metalloproteinase (MMPs), aspartic, and serine protease families (i.e. cathepsins, kallikrein-related peptidases) (FIG. 3B). From a matrisome proteomic analysis, proteases present in primary CRCs and their distant metastases were confirmed (e.g. MMP-7, -9, Cathepsin D, PLAU) (FIG. 13B) (Hynes et al. Cold Spring Harbor perspectives in biology 4, a004903 (2012); Naba et al. Molecular & cellular proteomics: MCP 11, M111 014647 (2012)). It was confirmed that these identified proteases were overexpressed in tumor-bearing lung tissue of the MC26 transplantation model compared to normal lung tissue (FIG. 13A, FIG. 13C). To identify peptide substrates specific to the selected proteases, 16 peptide sequences were screened against purified recombinant proteases and identified the top five substrates using a fluorogenic activity assay (FIG. 13D) (Dudani et al. Proceedings of the National Academy of Sciences of the United States of America 115, 8954-8959 (2018); Kirkpatrick et al. Science translational medicine 12(2020). These protease-activated peptides (PAP7, PAP9, PAP11, PAP13, PAP15) broadly cover metallo, serine, and aspartic protease activities (FIG. 13D), and were specifically cleaved by tumor tissue homogenates ex vivo with high predicted disease classification power, and thus were incorporated into the panel of DNA-SUBs for in vivo validation (FIG. 6B, FIGS. 14 and 3D).

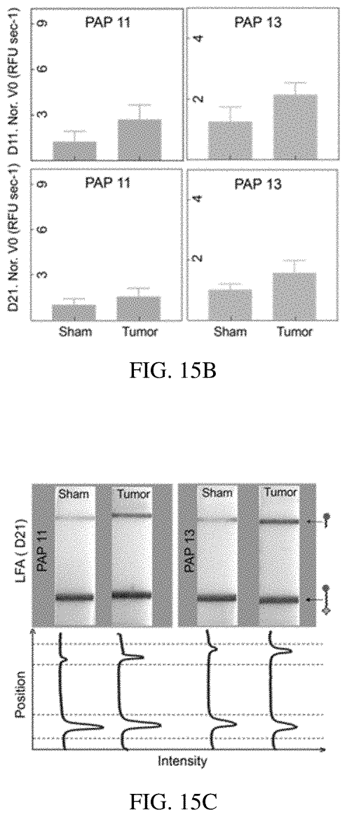

It was first shown that a DNA-barcoded, MMP-responsive SUB (DNA-PAP7-SUB) accumulated in the CRC lung tumor nodules following intravenous injection (FIG. 4C, FIG. 4B). The entire 5-plex of DNA-barcoded SUBs in vivo was then tested, with an emphasis on identifying reporters that differentiated mice bearing lung tumor nodules from the healthy control animals. The multiplexed DNA-SUBs was systemically administered to the two mouse cohorts over the course of tumor development, and quantified urinary DNA barcodes that were freed from the nanosensors one hour after injection. Urine samples were incubated with LbaCas12a-coupled with five different complementary crRNAs in multiple wells, and the trans-cleavage activity triggered by each DNA barcode was analyzed by tracking the cleavage kinetics of a fluorescence-quencher labeled reporter. It was found that the MMP-responsive sensor (DNA-PAP7-SUB) from this multiplexed panel succeeded in distinguishing tumor-bearing mice from healthy mice only 11 days after tumor inoculation when the tumor nodules were 1-2 mm. Some sensor (DNA-PAP9-SUB, DNA-PAP15-SUB) differences were amplified over time (FIG. 6C, FIG. 12A, FIG. 15B), and these sensors (PAP9 and PAP15) corresponded to peptides cleaved by serine and metalloproteases in vitro, and also produced distinct cleavage patterns when incubated with homogenates from either tumor-bearing or healthy lung tissues (FIG. 6B). Based on the ROC curve analysis, the sum of the metallo (PAP7, PAP15) and serine (PAP9) protease substrate signals significantly increased the classification power of the DNA-SUBs (combined sensors PAP7/9/15 AUC=0.94; PAP7 AUC=0.81; PAP9 AUC=0.88, PAP15 AUC=0.77; FIG. 6E). The 5-plex sensor panel was then combined with lateral flow detection for a visual readout that could enable PoC diagnostics. Using the same urine samples assayed in the aforementioned Cas12a kinetic cleavage reactions (FIG. 6C), the lateral flow assay was designed to read the cleavage of the FAM-poly(T)-biotin reporter at the optimized end timepoint. After the activation of LbaCas12a by incubating the enzyme with a specific crRNA and its complementary DNA barcodes in urine, the enzyme complex and FAM-poly(T)-biotin reporter were mixed and added onto an assigned location in a 96-well plate. A series of lateral flow strips were loaded onto the plates and the multiple-pot test paper results appeared in 5 min at room temperature (FIG. 6D). Consistent with the results in the fluorescent readout, the test paper ‘fingerprints’ revealed distinctions in the intensity of sample bands resulting from Cas12a activation of tumor-bearing mice and healthy mice (FIG. 6D, FIG. 15C). Notably, quantification of the sample band intensities exhibited disease classification power with multiple sensors (FIG. 6E), enabling a platform that is amenable to clinical translation due to its well-understood chemical composition and use of DNA multiplexing to overcome relatively low tumor accumulation, relative to ligand-targeted scaffolds.

### Example 3: Modification of crRNAs to Increase ssDNA Trans-Cleavage Activity of Cas12a

To increase the ssDNA trans-cleavage activity of Cas12a, modified crRNA is used to detect modified nucleic acid barcodes. Modification of crRNA enhances base pairing between the nucleic acid barcodes (e.g., DNA barcodes) and modified crRNA. Phosphorothioate modification, 2′-O-Methoxyethyl (2′-MOE) and/or other chemical modifications are incorporated into the crRNAs to enhance their stability or hybridization to DNA barcodes. Non-limiting examples of modified crRNAs are shown in Table 10. Using crRNA2 from Table 10 as an example, chemical modifications in the crRNA is incorporated into the complementary sequence to the DNA barcodes, fully or in part.

### Example 4: Design of Modified RNAs to Activate Cas13 Nucleases

RNA sequences are designed to create RNA barcodes that can activate Cas13 nucleases. The length of the RNA barcode for kidney filtration may be the same as that of the DNA barcode. A standard clinically applied antisense oligo (ASO)-like structure that has a central region of PS-modified bases, flanked on both sides by blocks of 2-MOE modifications, is used to increase the stability of RNAs in vivo. Non-limiting examples of modified RNAs that may be used to activate Cas13 nucleases is shown in Table 11.

### Example 5: Methods

### Synthesis of Protease-Activated Nanobody-DNA Barcode Conjugates

Protease-activated sequence (enzyme substrate) was genetically encoded in the C-terminus of the nanobody of interest. Recombinant nanobody expressed and purified from E. Coli was incubated at room temperature overnight in PIERCE™ immobilized TCEP disulfide reducing gel (7.5 v/v %) (ThermoFisher Scientific, MA, USA) to selectively reduce C-terminal cysteine. See, e.g., Kirley et al., Biochem Biophys Res Commun. 2016 Nov. 25; 480(4):752-757. The reduced C-terminal cysteine (1 eq.) was reacted with sulfo DBCO-maleimide crosslinker (4 eq.) (Click Chemistry Tools, AZ, USA) in PBS (pH 6.5, 1 mM EDTA) at room temperature for 6 h after which the excess crosslinker was removed with a disposable PD-10 desalting column (GE Healthcare Bio-Sciences, PA, USA). DBCO-functionalized nanobody was further refined via size exclusion chromatography with Superdex 200 Increase 10/300 GL column on AKTA fast protein liquid chromatography (FPLC) system. DNA reporter conjugation was performed by incubating DBCO-functionalized nanobody (1 eq.) with azide-functionalized DNA reporter (1.1 eq.) in PBS (pH 7.4) at room temperature for 24 h. Excess DNA reporter was removed via size exclusion chromatography as described above. The product was confirmed via SDS-PAGE analysis and quantified with a ThermoFisher Quant-iT Oligreen ssDNA Reagent.

### Lateral Flow Assay

Samples were prepared similarly and incubated for 30 minutes at 37° C. as in the fluorescence-based Cas12a activation assay described above, except 2× urine concentration was used. Reactions were then diluted by a factor of 4 into NEB Buffer and FAM/Biotin reporter (160 nM, IDT) into reaction volume of 100 ul. Solution was incubated at 37° C. for 1 or 3 hours and then 20 ul was added to 80 ul of HybriDetect 1 assay buffer (Milenia). HybriDetect 1 lateral flow strips were dipped into solution and resulting control and sample bands intensity were quantified using ImageJ.

### Animal Models

All animal studies were approved by the Massachusetts Institute of Technology (MIT) committee on animal care (MIT protocol 0417-025-20 & 0217-014-20). All experiments were conducted in compliance with institutional and national guidelines and supervised by Division of Comparative Medicine (DCM) of MIT staff. Female Balb/c and NCr nude mice were kept under standardized housing conditions. A sample size of minimum three mice per group was used for in vivo studies, numbers of animals per group were specified in the figure legends. Littermates of the same sex were randomly assigned to experimental and control groups. Establishment of the transplantation mouse models was described below.

### Cell Culture

Mouse cell lines MC26-LucF (carrying firefly luciferase, from Kenneth K. Tanabe Laboratory, Massachusetts General Hospital) was cultured in DMEM (Gibco) medium supplemented with 10% (v/v) fetal bovine serum (FBS)(Gibco), 1% (v/v) penicillin/streptomycin (CellGro) at 37° C. and in 5% CO2. Human cell lines PC-3 (ATCC® CRL-1435™) were grown in RPMI1640 (Gibco) supplemented with 10% (v/v) FBS and 1% (v/v) penicillin/streptomycin. RWPE1 cells were cultured in Keratinocyte serum-free medium (Gibco) supplemented with 2.5 μg Human Recombinant EGF (rhEGF) and 25 mg Bovine Pituitary Extract (BPE). All cell lines tested negative for mycoplasma contamination.

### Peptide, Oligonucleotides and Peptide-Oligonucleotide Conjugates Synthesis and Characterization

All peptides were chemically synthesized by CPC Scientific, Inc. All oligonucleotides were synthesized by Integrated DNA Technologies, Inc. (IDT). Peptide-oligonucleotides conjugates were generated by copper-free click chemistry. The conjugates were purified on Agilent 1100 HPLC. Mass analysis of the conjugates was performed on a Bruker model MicroFlex MALDI-TOF (matrix-absorption laser desorption instrument time-of-flight). Sequences of all molecules are listed in Tables 5 and 7.

### Cas12a Fluorescent Cleavage Assay

LbCas12a (final concentration 100 nM, New England Biolabs) was incubated with 1× NEB BUFFER™ 2.1, crRNA (250 nM, IDT) and complementary DNA activators (4 nM unless specifically described, IDT, in solution or spiked in urine) or urine samples collected from experimental animals at 37° C. for 30 min. Reactions were diluted by a factor of 4 into 1×NEB BUFFER™ 2.1 and ssDNA T₁₀ F-Q reporter substrate (30 pmol, IDT) into a reaction volume of 60 μL per well. LbCas12a activation was detected at 37° C. every 2 min for 3 hours by measuring fluorescence with plate reader Tecan Infinite Pro M200 (λex: 485 nm and λem: 535 nm). Sequences of all oligonucleotides are listed in Table 5. Fluorescence for background conditions (either no DNA activator input or no crRNA conditions) were run with each assay to generate background fluorescence as negative controls. Cas12a ssDNase activity was calculated from the kinetics curve generated on the plate reader, and reflected by the initial reaction velocity (V₀), which refers to the slope of the curve at the beginning of a reaction.

Cas12a Cleavage Assay with Lateral Flow Readout

Samples were prepared similarly and incubated for 30 min at 37° C. as in Cas12a activation assay described above. Reactions were then diluted by a factor of 4 into 1×NEB BUFFER™ 2.1 and ssDNA T₁₀ FAM/Biotin reporter substrate (1 pmol, IDT) into reaction volume of 100 μl. Reactions were allowed to proceed at 37° C. for 1-3 hours unless otherwise indicated, and then 20 μl was added to 80 μl of HybriDetect 1 assay buffer (Milenia). HybriDetect 1 lateral flow strips were dipped into solution and intensity of bands was quantified in ImageJ.

Characterization of DNA Activator Concentration or Length for Cas12a ssDNase Activity

To identify the optimal length for detection with Cas12a, truncated native and modified DNA activator lengths from 15-34 nt were tested and it was found that in the Cas12a fluorescent cleavage assay described above, Cas12a had a peak sensitivity at a native DNA activator length of 24-mer, in which contains PAM sequence and complementary sequence of crRNA. To further explore the robustness of modified DNA activator in vivo, phosphorothioate-modified DNA activators with different lengths were injected at 1 nmol in Balb/c mice, respectively, and urine samples were collected after 1 h of injection. Urine samples were applied as DNA activators in the Cas12a fluorescent cleavage assay, Cas12a ssDNase activity triggered by each DNA activator was normalized to that of the 24-mer modified DNA activator.

### Cloning and Expression of Recombinant Nanobodies

Double-stranded GB LOCKS® gene fragments encoding nanobody of interest with flanking NcoI and BlpI restriction sites, as listed below, were ordered from Integrated DNA Technologies (IA, USA). The gene fragments were cloned into Novogen pET-28a(+) expression vector at NcoI and BlpI restriction sites and transformed into SHUFFLE® T7 competent E. coli. (New England Biolabs Inc., MA, USA). Bacteria colonies encoding the correct gene inserts were confirmed with Sanger sequencing. For subsequent recombinant protein production, a 500 mL secondary culture of SHUFFLE® T7 competent E. coli. encoding nanobody gene of interest was grown in kanamycin-supplemented LB broth at 37° C. from an overnight 3-mL primary culture until optical density at 600 nm (OD600) reached about 0.6-0.8. Nanobody expression was then induced with an addition of isopropyl β-D-1-thiogalactopyranoside (IPTG) (0.4 mM final concentration). The culture was incubated at 27° C. for 24 h after which bacteria were pelleted and stored at −80° C. Subsequently, the bacteria pellet was thawed on a water bath at 37° C. and lysed with B-PER™ complete bacteria protein extraction reagent (ThermoFisher Scientific, MA, USA). The released nanobody was purified via standard immobilized metal affinity chromatography (IMAC) with Ni-NTA agarose (Qiagen, MD, USA). The product was confirmed via SDS-PAGE analysis.

Synthesis of DNA-Encoded Synthetic Urine Biomarker with a Nanobody Core

Nanobody (2 mg) was incubated at room temperature overnight in PIERCE™ immobilized TCEP disulfide reducing gel (7.5 v/v %) (ThermoFisher Scientific, MA, USA) to selectively reduce C-terminal cysteine following a previously established protocol 31. The reduced C-terminal cysteine (1 eq.) was reacted with sulfo DBCO-maleimide crosslinker (4 eq.) (Click Chemistry Tools, AZ, USA) in PBS (pH 6.5, 1 mM EDTA) at room temperature for 6 h after which the excess crosslinker was removed with a disposable PD-10 desalting column (GE Healthcare Bio-Sciences, PA, USA). DBCO-functionalized nanobody was further refined on the fast-protein liquid chromatography (FPLC, GE Healthcare). DNA reporter conjugation was performed by incubating DBCO-functionalized nanobody (1 eq.) with azide-functionalized DNA reporter (1.1 eq.) in PBS (pH 7.4) at room temperature for 24 h. Excess DNA reporter was removed via size exclusion chromatography as described above. The product was confirmed via SDS-PAGE analysis and quantified with QUANT-IT™ OLIGREEN™ ssDNA Assay Kit.

Synthesis of DNA-Encoded Synthetic Urine Biomarkers with Polymeric Cores

Multivalent PEG (40 kDa, eight-arm) containing maleimide-reactive handles (JenKem Technology) was dissolved in 100 mM phosphate buffer (pH 7.0) and filtered (pore size: 0.2 μm). After filtration, the cysteine terminated peptide-DNA conjugates were added at 2-fold molar excess to the PEG and reacted for at least 4 h at room temperature. Unconjugated molecules were separated using size exclusion chromatography with Superdex 200 Increase 10/300 GL column on AKTA fast protein liquid chromatography (FPLC, GE Healthcare). The purified nanosensors were concentrated by spin filters (MWCO=10 kDa, Millipore). Concentration of the nanosensor was quantified QUANT-IT™ OLIGREEN™ ssDNA Assay Kit (ThermoFisher), fluorescence was read on a Tecan Infinite Pro M200 plate reader Quant-iT Oligreen ssDNA Reagent at λex: 485 nm and λem: 535 nm). Particles were stored at 4° C. in PBS. Dynamic light scattering (Zeta Sizer Nanoseries, Malvern Instruments, Ltd) was used to characterized the hydrodynamic diameter of the nanoparticles.

### Transcriptomic and Proteomic Analysis

RNA-Seq data of human colon adenocarcinoma (285 tumor samples vs 41 normal tissue samples) were obtained from the TCGA Research Network (cancergenome.nih.gov). Differential expression analyses were carried out by DESeq2 1.10.1. Proteomic data on the composition of extracellular matrix in human colon cancers and normal colon tissues were obtained by mass spectrometry analysis of ECM components and available from Matrisome (matrisomeproject.mit.edu/).

### Establishment of the Animal Models and Urine Collection

Balb/c female mice (6-8 wks of age) were inoculated by intravenous (IV) injection with murine cell lines (100 k cells/mouse, MC26-Fluc) expressing firefly luciferase. Tumor progression was monitored weekly using IVIS Imaging Systems (IVIS, PerkinElmer). To establish the prostate cancer xenograft model, NCr nude female mice (4-5 wks of age) were inoculated with human PC-3 cell lines (5 million cells per flank, 2 flanks per mouse). Cells were prepared in 30% CORNING™ MATRIGEL™ Membrane Matrix (Thermo Fisher Scientific) and low-serum media (OPTI-MEM®, Gibco). Tumors were measured weekly and experiments were conducted once flank tumors reached adequate size, which was approximately 5 mm in length or width (˜200 mm3) or three weeks after inoculation. Tumor volume was calculated by caliper measurement of the length and width of the flank; volume calculation followed the equation fx=IF (length>width, (width\hat{ }2*length)/2, (length\hat{ }2*width)/2). For urine analysis, after injection with synthetic biomarkers, mice were placed into custom housing with a 96-well plate base for urine collection. The bladders were voided to collect between 100-200 μL of urine at 1 h post injection. By the end time point of each study, mice were sacrificed and tumor tissues were collected for further analysis.

### Analysis of Urinary DNA Barcode Activated Cas12a Cleavage Assay

ssDNAs (1 nmol), 5-plex DNA-barcoded PEG sensors (0.2 nmol each by DNA barcode concentration, 1 nmol by DNA barcode concentration in total), or DNA-barcoded nanobody sensors (1 nmol by DNA barcode concentration) were injected into experimental mice via intravenous injection. Urine samples were collected after 1 h and used as DNA activator in Cas12a fluorescent cleavage assay described above. The initial reaction velocity (V₀) is determined from the slope of the curve at the beginning of a reaction. Mean normalization was performed on V₀ values to account for animal-to-animal variation in urine concentration. In the Cas12a cleavage assay with fluorescent reporter, Y axis represents MeanNorm V_{0 Tumor-bearing animals}/MeanNorm V_{0 control animals}. Then the same urine sample were utilized to perform the Cas12a cleavage assay with LFA readout. Resulting paper strips were aligned and scanned simultaneously, intensity of control and sample bands were quantified from the scanned images in ImageJ.

### Biodistribution and Pharmacokinetics Studies

Studies were performed in experimental animals with near-infrared dye labeled agents to minimize interference from autofluorescent background. Balb/c mice were intravenously injected with Cy5-labeled modified or native DNA molecules at 1 nmol per mouse, n=3 per condition. Urine samples from each mouse was collected at 30 min, 1, 2, 3, 4 hours after injection. C-met nanobodies were coupled with Sulfo-Cyanine7 NHS ester (Lumiprobe), reacted overnight, purified by spin filtration and injected into PC-3 tumor-bearing nude mice (1.5 nmol dye eq. of protein) via i.v. injection. After 24 hours, mice were euthanized and necropsy was performed to remove the tumors, lungs, heart, kidneys, liver, and spleen. Urine, blood and organs were scanned using IVIS Imaging Systems and ODYSSEY® CLx (LI-COR). Organ fluorescence was quantified in ImageStudio of ODYSSEY® CLx. Blood circulatory kinetics were monitored in Balb/c mice by serial blood draws at 10 min, 30 min, 120 min and 180 min after i.v. injection of Cy5-labeled DNA or PEG at 1 nmol dye per mouse. Blood for pharmacokinetics measurements was collected using tail vain bleeds. Blood was diluted in PBS with 5 mM EDTA to prevent clotting, centrifuged for 5 min at 5,000×g, and fluorescent reporter concentration was quantified in 384-well plates relative to standards (LI-COR ODYSSEY® CLx).

### Histology, Immunohistochemistry (IHC) and Immunofluorescence (IF) Studies

Paraffin-embedded tissues were preserved in 4% paraformaldehyde (PFA) overnight and stored in 70% ethanol prior to embedding into paraffin. Snap-frozen tissues were preserved in 2% PFA for two hours, stored in 30% sucrose overnight and frozen in optimum cutting temperature (OCT) compound at −80° C. Snap-frozen lungs were processed through intratracheal injection of 50:50 OCT in PBS immediately after animal euthanasia. The lungs were slowly frozen with OCT embedding in isopentane liquid nitrogen bath. Samples were sectioned into 6 μm slices and stained for further analysis. For IHC studies, slides were stained with primary antibodies in accordance with manufacturer instructions, followed by HRP secondaries. For IF studies, after blocking with 5% goat serum, 2% BSA, 0.1% Triton-X 100 in PBS for 1 h, sections were stained with a primary antibody in 1% BSA in PBS overnight at 4° C. AlexaFluor conjugated secondary antibodies were incubated at 1 μg/mL in 1% BSA in PBS for 30 min at RT. Slides were sealed with ProLong Antifade Mountants (Thermo Scientific). Slides were digitized and analyzed using an 3D Histech P250 High Capacity Slide Scanner (Perkin Elmer). Antibodies and dilutions used were listed in Table 8.

### RNA Extraction and RT-qPCR

PC-3 and RWPE1 cells were cultured and collected after trypsinization. Tissue samples were collected by necropsy after mice were euthanized and were immediately kept in RNAlater RNA Stabilization Reagent (Qiagen, Inc.). RNA from cell pallets or cryogrounded tissue samples was extracted using RNeasy Mini Kit (Qiagen, Inc.). RNA was reverse transcribed into cDNA using BioRad iScript Reverse Transcription Supermix on a Bio-Rad iCycler. qPCR amplification of the cDNA was measured after mixing with Taqman gene expression probes and Applied Biosystems TaqMan Fast Advanced Master Mix (Thermo Scientific) according to manufactory's instruction. qPCR was performed on a CFX96 Real Time System C1000 Thermal Cycler from Bio-Rad.

### Recombinant Protease Substrate Cleavage Assay

Fluorogenic protease substrates with fluorophore (FAM) and quencher (CPQ2) were synthesized by CPC Scientific Inc. Recombinant proteases were purchased from Enzo Life Sciences and R&D Systems. Assays were performed in the 384-well plate in triplicate in enzyme-specific buffer with peptides (1 μM) and proteases (40 nM) in 30 μL at 37° C. Fluorescence was measured at Ex/Em 485/535 nm using a Tecan Infinite 200pro microplate reader (Tecan). Signal increase at 60 min was used across conditions. Enzymes and buffer conditions were listed in Table 9.

### Protein Extraction and Tissue Lysate Proteolytic Cleavage Assay

Tissue samples were homogenized in PBS and centrifuged at 4° C. for 5 min at 6,000×g. Supernatant was further centrifuged at 14,000×g for 25 min at 4° C. Protein concentration was measured using ThermoFisher BCA Protein Assay Kit and prepared at 2 mg/mL prior to assay. Assays were performed in the 384-well plate in triplicate in enzyme-specific buffer with peptides (1 μM) and cell lysates (0.33 mg/mL) in 30 μL at 37° C. Fluorescence was measured at Ex/Em 485/535 nm using a Tecan Infinite 200pro microplate reader (Tecan). Signal increase at 60 min was used across conditions.

### Quantification and Statistical Analysis

Statistical analyses were conducted in GraphPad Prism (Version 8.4). Data were presented as means with standard error of the mean (SEM). Differences between groups were assessed using parametric and non-parametric group comparisons when appropriate with adjustment for multiple hypothesis testing. Results were tested for statistical significance by Student's t-test (parametric) or Mann-Whitney U test (nonparametric) for two group comparisons and ANOVA for multiple group comparisons. Sample sizes and statistical test are specified in the brief description of the drawings.

TABLE 5
Exemplary Nucleic Acid Sequences

| Name of | |
|---|---|
| oligo | Sequence (5′→3′) |
| crRNA 1 | UAAUUUCUACUAAGUGUAGAUCGUCGCCGUCCAGCUCGACC (SEQ |
| | ID NO: 9) |
| crRNA 2 | UAAUUUCUACUAAGUGUAGAUGAUCGUUACGCUAACUAUGA (SEQ |
| | ID NO: 10) |
| crRNA 3 | UAAUUUCUACUAAGUGUAGAUCCUGGGUGUUCCACAGCUGA (SEQ |
| | ID NO: 11) |
| crRNA 5 | UAAUUUCUACUAAGUGUAGAUCTGTGTTTATCCGCTCACAA (SEQ |
| | ID NO: 12) |
| crRNA 6 | UAAUUUCUACUAAGUGUAGAUUGAAGUAGAUAUGGCAGCAC (SEQ |
| | ID NO: 13) |
| crRNA 7 | UAAUUUCUACUAAGUGUAGAUACAAUAUGUGCUUCUACACA (SEQ |
| | ID NO: 14) |
| ssDNA | TAGCATTCCACAGACAGCCCTCATAGTTAGCGTAACGATCTAAAGTT |
| | TTGTCGTC (SEQ ID NO: 15) |
| Mod | T*A*G*C*A*T*T*C*C*A*C*A*G*A*C*A*G*C*C*C*T*C*A*T*A*G*T* |
| ssDNA | T*A*G*C*G*T*A*A*C*G*A*T*C*T*A*A*A*G*T*T*T*T*G*T*C*G*T* |
| | C (SEQ ID NO: 16) |
| dsDNA- | TAGCATTCCACAGACAGCCCTCATAGTTAGCGTAACGATCTAAAGTT |
| strand 1 | TTGTCGTC (SEQ ID NO: 17) |
| dsDNA- | GACGACAAAACTTTAGATCGTTACGCTAACTATGAGGGCTGTCTGTG |
| strand 2 | GAATGCTA (SEQ ID NO: 18) |
| (comple- | |
| mentary) | |
| Mod DNA 1 | G*G*T*C*G*A*G*C*T*G*G*A*C*G*G*C*G*A*C*G (SEQ ID NO: 19) |
| Mod DNA 2 | T*C*A*T*A*G*T*T*A*G*C*G*T*A*A*C*G*A*T*C (SEQ ID NO: 20) |
| Mod DNA 3 | T*C*A*G*C*T*G*T*G*G*A*A*C*A*C*C*C*A*G*G (SEQ ID NO: 21) |
| Mod DNA 4 | G*A*G*T*A*A*C*A*G*A*C*A*T*G*G*A*C*C*A*T*C*A*G (SEQ ID |
| | NO: 22) |
| Mod DNA 5 | T*T*G*T*G*A*G*C*G*G*A*T*A*A*A*C*A*C*A*G (SEQ ID NO: 23) |
| Mod DNA 6 | G*T*G*C*T*G*C*C*A*T*A*T*C*T*A*C*T*T*C*A (SEQ ID NO: 24) |
| Mod DNA 7 | T*G*T*G*T*A*G*A*A*G*C*A*C*A*T*A*T*T*G*T (SEQ ID NO: 25) |
| Dye-DNA 2 | /Cy5/TCATAGTTAGCGTAACGATC (SEQ ID NO: 26) |
| Dye-Mod | /Cy5/T*C*A*T*A*G*T*T*A*G*C*G*T*A*A*C*G*A*T*C (SEQ ID NO: |
| DNA 2 | 27) |
| 10 mer | CGTAACGATC (SEQ ID NO: 28) |
| 15 mer | GTTAGCGTAACGATC (SEQ ID NO: 29) |
| 20 mer | TCATAGTTAGCGTAACGATC (SEQ ID NO: 30) |
| 24 mer | TCATAGTTAGCGTAACGATCTAAA (SEQ ID NO: 31) |
| 26 mer | CAGCCCTCATAGTTAGCGTAACGATC (SEQ ID NO: 32) |
| 30 mer | CAGCCCTCATAGTTAGCGTAACGATCTAAA (SEQ ID NO: 33) |
| 34 mer | GACAGCCCTCATAGTTAGCGTAACGATCTAAAGT (SEQ ID NO: 34) |
| Mod 10 mer | C*G*T*A*A*C*G*A*T*C (SEQ ID NO: 35) |
| Mod 15 mer | G*T*T*A*G*C*G*T*A*A*C*G*A*T*C (SEQ ID NO: 36) |
| Mod 20 mer | T*C*A*T*A*G*T*T*A*G*C*G*T*A*A*C*G*A*T*C (SEQ ID NO: 37) |
| Mod 24 mer | T*C*A*T*A*G*T*T*A*G*C*G*T*A*A*C*G*A*T*C*T*A*A*A (SEQ ID |
| | NO: 38) |
| Mod 26 mer | C*A*G*C*C*C*T*C*A*T*A*G*T*T*A*G*C*G*T*A*A*C*G*A*T*C |
| | (SEQ ID NO: 39) |
| Mod 30 mer | C*A*G*C*C*C*T*C*A*T*A*G*T*T*A*G*C*G*T*A*A*C*G*A*T*C*T* |
| | A*A*A (SEQ ID NO: 40) |
| Mod 34 mer | G*A*C*A*G*C*C*C*T*C*A*T*A*G*T*T*A*G*C*G*T*A*A*C*G*A*T* |
| | C*T*A*A*A*G*T (SEQ ID NO: 41) |
| Mod DNA 1 | G*G*T*C*G*A*G*C*T*G*G*A*C*G*G*C*G*A*C*G\DBCO (SEQ ID |
| 3′-DBCO | NO: 42) |
| Mod DNA 2 | T*C*A*T*A*G*T*T*A*G*C*G*T*A*A*C*G*A*T*C\DBCO (SEQ ID NO: |
| 3′-DBCO | 43) |
| Mod DNA 3 | T*C*A*G*C*T*G*T*G*G*A*A*C*A*C*C*C*A*G*G\DBCO (SEQ ID |
| 3′-DBCO | NO: 44) |
| Mod DNA 4 | G*A*G*T*A*A*C*A*G*A*C*A*T*G*G*A*C*C*A*T*C*A*G\DBCO |
| 3′-DBCO | (SEQ ID NO: 45) |
| Mod DNA 5 | T*T*G*T*G*A*G*C*G*G*A*T*A*A*A*C*A*C*A*G\DBCO (SEQ ID |
| 3′-DBCO | NO: 46) |
| Mod DNA 6 | G*T*G*C*T*G*C*C*A*T*A*T*C*T*A*C*T*T*C*A\DBCO (SEQ ID NO: |
| 3′-DBCO | 47) |
| Mod DNA 7 | T*G*T*G*T*A*G*A*A*G*C*A*C*A*T*A*T*T*G*T\DBCO (SEQ ID NO: |
| 3′-DBCO | 48) |
| Mod DNA 2 | T*C*A*T*A*G*T*T*A*G*C*G*T*A*A*C*G*A*T*C\Azide (SEQ ID NO: |
| 3′-Azide | 49) |

Table 5 Symbol Key:
*, phosphorothioate modification
DBCO, Dibenzocyclooctyne
Cy5, Cyanine 5 dye

TABLE 6
Activation of Cas12a with native and modified
DNA oligos in vitro and in vivo

| | Modified | Modified | Native | Native |
|---|---|---|---|---|
| | DNA in vitro | DNA in vivo | DNA in vitro | DNA in vivo |
| Oligo | (V₀)* | (V₀) | (V₀) | (V₀) |
| 10 mer | 0.01 | 0.01 | 0.00 | 0.00 |
| 15 mer | 0.01 | 0.01 | 0.00 | 0.00 |
| 20 mer | 6.29 | 1.82 | 7.67 | 0.02 |
| 24 mer | 8.91 | 0.92 | 10.94 | 0.00 |
| 26 mer | 5.25 | 0.60 | 6.09 | 0.01 |
| 30 mer | 4.65 | 0.34 | 4.98 | 0.00 |
| 34 mer | 4.55 | 0.38 | 3.32 | 0.01 |

*The initial reaction velocity (V₀) refers to the slope of the curve at the beginning of a reaction.

TABLE 7
Exemplary Peptide and Protein sequences

| Name of | |
|---|---|
| peptide | Sequence (N→C) |
| Q7-click | K(N3)-ANP-GGPLGVRGKGGC (SEQ ID NO: 50) |
| Q9-click | K(N3)-ANP-GG-DPhe-PRSGGC (SEQ ID NO: 51) |
| PQ2-click | K(N3)-ANP-GGGSGRSANAKGGC (SEQ ID NO: 52) |
| PQ12-click | K(N3)-ANP-GGVPRGGC (SEQ ID NO: 53) |
| PQ19-click | K(N3)-ANP-GPVPLSLVMGGC (SEQ ID NO: 54) |
| FRET-PAP1 | 5FAM-GGPQGIWGQK(CPQ2)-PEG2-GC (SEQ ID NO: 55) |
| FRET-PAP2 | 5FAM-GGLVPRGSGK(CPQ2)-PEG2-GC (SEQ ID NO: 56) |
| FRET-PAP3 | 5FAM-GGPVGLIGK(CPQ2)-PEG2-GC (SEQ ID NO: 57) |
| FRET-PAP4 | 5FAM-GGPWGIWGQGK(CPQ2)-PEG2-GC (SEQ ID NO: 58) |
| FRET-PAP5 | 5FAM-GGPVPLSLVMK(CPQ2)-PEG2-GC (SEQ ID NO: 59) |
| FRET-PAP6 | 5FAM-GGPLGLRSWK(CPQ2)-PEG2-GC (SEQ ID NO: 60) |
| FRET-PAP7 | 5FAM-GGPLGVRGKK(CPQ2)-PEG2-GC (SEQ ID NO: 61) |
| FRET-PAP8 | 5FAM-GGf-Pip-RSGGGK(CPQ2)-PEG2-GC (SEQ ID NO: 62) |
| FRET-PAP9 | 5FAM-GGfPRSGGGK(CPQ2)-PEG2-GC (SEQ ID NO: 63) |
| FRET-PAP10 | 5FAM-GGf-Pip-KSGGGK(CPQ2)-PEG2-GC (SEQ ID NO: 64) |
| FRET-PAP11 | 5FAM-GGGSGRSANAKG-K(CPQ2)-PEG2-GC (SEQ ID NO: 65) |
| FRET-PAP12 | 5FAM-GILSRIVGGG-K(CPQ2)-PEG2-GC (SEQ ID NO: 66) |
| FRET-PAP13 | 5FAM-GGVPRGG-K(CPQ2)-PEG2-GC (SEQ ID NO: 67) |
| FRET-PAP14 | 5FAM-GSGSKIIGGG-K(CPQ2)-PEG2-GC (SEQ ID NO: 68) |
| FRET-PAP15 | 5FAM-GPVPLSLVMG-K(CPQ2)-PEG2-GC (SEQ ID NO: 69) |
| FRET-PAP16 | 5FAM-GGLGPKGQTGK(CPQ2)-kk-PEG2-C (SEQ ID NO: 70) |
| cMET | MEVQLVESGGGLVQPGGSLRLSCAASGFILDYYAIGWFRQAPGKERE |
| nanobody | GVLCIDASDDITYYADSVKGRFTISRDNAKNTVYLQMNSLKPEDTGV |
| | YYCATPIGLSSSCLLEYDYDYWGQGTLVTVSSGSHHHHHHSPSTPPTP |
| | SPSTPPGSGRSANAKGGGSC (SEQ ID NO: 71) |
| GFP | MAQVQLVESGGRLVQAGDSLRLSCAASGRTFSTSAMAWFRQAPGRE |
| nanobody | REFVAAITWTVGNTILGDSVKGRFTISRDRAKNTVDLQMDNLEPEDT |
| | AVYYCSARSRGYVLSVLRSVDSYDYWGQGTQVTVSGSHHHHHHSPS |
| | TPPTPSPSTPPGSGRSANAKGGGSC |
| | (Clone LaG-16) (SEQ ID NO: 72) |
| | (Fridy et al. Nature methods 11, 1253-1260 (2014). |
| Cas12a | AASKLEKFTNCYSLSKTLRFKAIPVGKTQENIDNKRLLVEDEKRAEDY |
| | KGVKKLLDRYYLSFINDVLHSIKLKNLNNYISLFRKKTRTEKENKELE |
| | NLEINLRKEIAKAFKGAAGYKSLFKKDIIETILPEAADDKDEIALVNSF |
| | NGFTTAFTGFFDNRENMFSEEAKSTSIAFRCINENLTRYISNMDIFEKV |
| | DAIFDKHEVQEIKEKILNSDYDVEDFFEGEFFNFVLTQEGIDVYNAIIG |
| | GFVTESGEKIKGLNEYINLYNAKTKQALPKFKPLYKQVLSDRESLSFY |
| | GEGYTSDEEVLEVFRNTLNKNSEIFSSIKKLEKLFKNFDEYSSAGIFVK |
| | NGPAISTISKDIFGEWNURDKWNAEYDDIHLKKKAVVTEKYEDDRR |
| | KSFKKIGSFSLEQLQEYADADLSVVEKLKEIIIQKVDEIYKVYGSSEKL |
| | FDADFVLEKSLKKNDAVVAIMKDLLDSVKSFENYIKAFFGEGKETNR |
| | DESFYGDFVLAYDILLKVDHIYDAIRNYVTQKPYSKDKFKLYFQNPQF |
| | MGGWDKDKETDYRATILRYGSKYYLAIMDKKYAKCLQKIDKDDVN |
| | GNYEKINYKLLPGPNKMLPKVFFSKKWMAYYNPSEDIQKIYKNGTFK |
| | KGDMFNLNDCHKLIDFFKDSISRYPKWSNAYDFNFSETEKYKDIAGF |
| | YREVEEQGYKVSFESASKKEVDKLVEEGKLYMFQIYNKDFSDKSHGT |
| | PNLHTMYFKLLFDENNHGQIRLSGGAELFMRRASLKKEELVVHPANS |
| | PIANKNPDNPKKTTTLSYDVYKDKRFSEDQYELHIPIAINKCPKNIFKI |
| | NTEVRVLLKHDDNPYVIGIDRGERNLLYIVVVDGKGNIVEQYSLNEII |
| | NNFNGIRIKTDYHSLLDKKEKERFEARQNWTSIENIKELKAGYISQVV |
| | HKICELVEKYDAVIALEDLNSGFKNSRVKVEKQVYQKFEKMLIDKLN |
| | YMVDKKSNPCATGGALKGYQITNKFESFKSMSTQNGFIFYIPAWLTS |
| | KIDPSTGFVNLLKTKYTSIADSKKFISSFDRIMYVPEEDLFEFALDYKN |
| | FSRTDADYIKKWKLYSYGNRIRIFAAAKKNNVFAWEEVCLTSAYKEL |
| | FNKYGINYQQGDIRALLCEQSDKAFYSSFMALMSLMLQMRNSITGRT |
| | DVDFLISPVKNSDGIFYDSRNYEAQENAILPKNADANGAYNIARKVL |
| | WAIGQFKKAEDEKLDKVKIAISNKEWLEYAQTSVK (SEQ ID NO: 73) |

### Table 7 Symbol Key:

Upper case, L-form amino acid;
Lower case, D-form amino acid;
Underlined, rigid linker sequence;
Bolded, PLAU substrate sequence (Dudani et al. Proceedings of the National Academy of Sciences of the United States of America 115, 8954-8959 (2018).
N3, Azide side chain; ANP, photocleavable linker; SFAM, N-terminal Fluorescein fluorophore

TABLE 8
List of exemplary primary antibodies

| Antibody | Cat# | Manufacturer | Application | Dilution |
|---|---|---|---|---|
| CTSD | ab75852 | Abcam | IF | 1:100 |
| MMP3 | ab194717 | Abcam | IF | 1:200 |
| MMP7 | ab5706 | Abcam | IF | 1:100 |
| MMP9 | ab38898 | Abcam | IHC, IF | 1:200 |
| PEG | ab190652 | Abcam | IHC | 1:200 |
| PLAU | ab24121 | Abcam | IHC | 1:100 |
| c-Met | ab51067 | Abcam | IHC | 1:100 |
| Cyanine | sc-166895 | Santa Cruz | IF | 1:100 |

TABLE 9
List of exemplary buffers for proteolytic cleavage assays

| Enzyme | Manufacturer | Buffer |
|---|---|---|
| MMPs | Enzo | 50 mM TRIS, 10 mM CaCl₂, |
| | | 300 mM NaCl, 20 μM ZnCl₂, |
| | | 0.02% Brij-35, 1% BSA, pH 7.5 |
| ADAMs | Enzo | 10 mM HEPES, 100 mM NaCl, 0.01% |
| | | Brij-35, 1% BSA, pH 7.4 |
| Cathepsin B | R&D | 25 mM MES, 5 mM DTT, pH 5.0 |
| Cathepsin D | R&D | 0.1M NaOAc, 0.2M NaCl, pH 3.5 |
| Cathepsin E | R&D | 0.1M NaOAc, 0.5M NaCl, pH 3.5 |
| Cathepsin K | Enzo | 50 mM NaOAc, 1 mM DTT, pH 5.5 |
| Cathepsin L | R&D | 50 mM MES, 5 mM DTT, 1 mM EDTA, |
| | | 0.005% (w/v) Brij-35, pH 6.0 |
| Cathepsin S | R&D | 50 mM NaOAc, 5 mM DTT, 250 mM |
| | | NaCl, pH 4.5 |
| uPA/PLAU | R&D | 50 mM Tris, 0.01% Tween 20, 1% |
| | | BSA, pH 7.4 |

TABLE 10
Non-limiting examples of modified crRNA sequences.

| Name | | SEQ |
|---|---|---|
| of | | ID |
| oligo | Sequence (5′→3′) | NO: |
| crRNA 2- | UAAUUUCUACUAAGUGUAGAUG*A*U*C*G | 85 |
| PS-1 | *U*U*A*C*G*C*U*A*A*C*U*A*U*G*A | |
| crRNA 2- | UAAUUUCUACUAAGUGUAGAUGAUCGUUAC | 86 |
| PS-2 | G*C*U*A*A*C*U*A*U*G*A | |
| crRNA 2- | UAAUUUCUACUAAGUGUAGAUGAUCGUUAC | 87 |
| PS-3 | GCUAACU*A*U*G*A | |
| crRNA 2- | UAAUUUCUACUAAGUGUAGAUmGmAmUmCm | 88 |
| 2MOE-1 | GmUmUmAmCmGmCmUmAmAmCmUmAmUmGm | |
| | A | |
| crRNA 2- | UAAUUUCUACUAAGUGUAGAUGAUCGUUAC | 89 |
| 2MOE-2 | GmCmUmAmAmCmUmAmUmGmA | |
| crRNA 2- | UAAUUUCUACUAAGUGUAGAUGAUCGUUAC | 90 |
| 2MOE-3 | GCUAACUmAmUmGmA | |

Table 10 Symbol Key:
*, phosphorothioate modification
m, 2′-O-Methoxyethyl (2′-MOE) modifications

TABLE 11
Non-limiting examples of modified RNA barcode sequences.

| | | SEQ ID |
|---|---|---|
| Name of oligo | Sequence (5′→ 3′) | NO: |
| Mod RNA 1 | G*G*U*C*G*AmGmCmUmGmGmAmCmGmGmC*G*A*C*G\DBCO | 91 |
| 3′-DBCO | | |
| Mod RNA 2 | U*C*A*U*A*GmUmUmAmGmCmGmUmAmAmC*G*A*U*C\DBCO | 92 |
| 3′-DBCO | | |
| Mod RNA 3 | U*C*A*G*C*UmGmUmGmGmAmAmCmAmCmC*C*A*G*G\DBCO | 93 |
| 3′-DBCO | | |
| Mod RNA 4 | G*A*G*U*A*AmCmAmGmAmCmAmUmGmGmA*mCmCmA*U*C*A*G\ | 94 |
| 3′-DBCO | DBCO | |
| Mod RNA 5 | U*U*G*U*G*AmGmCmGmGmAmUmAmAmAmC*A*C*A*G\DBCO | 95 |
| 3′-DBCO | | |
| Mod RNA 6 | G*U*G*C*U*GmCmCmAmUmAmUmCmUmAmC*U*U*C*A\DBCO | 96 |
| 3′-DBCO | | |
| Mod RNA 7 | U*G*U*G*U*AmGmAmAmGmCmAmCmAmUmA*U*U*G*U\DBCO | 97 |
| 3′-DBCO | | |

Table 11 Symbol Key:
*, phosphorothioate modification
m, 2′-O-Methoxyethyl (2′-MOE) modifications

### Additional Embodiments

Paragraph 1. A sensor comprising a scaffold linked to a modified nucleic acid barcode that is capable of being released from the sensor when exposed to an enzyme present in a subject.
Paragraph 2. The sensor of paragraph 1, wherein the modified nucleic acid barcode comprises a modified internucleoside linkage, a modified nucleotide, and/or a terminal modification.
Paragraph 3. The sensor of paragraph 2, wherein the modified internucleoside linkage is selected from a phosphorothioate linkage or a boranophosphate linkage.
Paragraph 4. The sensor of any one of paragraphs 1-3, wherein the modified nucleic acid barcode comprises at least two different modifications.
Paragraph 5. The sensor of any one of paragraphs 1-4, wherein the modified nucleic acid barcode comprises a modified sugar moiety and/or a modified base.
Paragraph 6. The sensor of paragraph 5, wherein the modified sugar moiety comprises a 2′-OH group modification and/or a bridging moiety.
Paragraph 7. The sensor of paragraph 6, wherein the 2′-OH group modification is selected from the group consisting of 2′-O-Methyl (2′-O-Me), 2′-Fluoro (2′-F), and 2′-O-methoxy-ethyl (2′-O-MOE).
Paragraph 8. The sensor of any one of paragraphs 5-7, wherein the modified base is a deoxyuridine (dU), a 5-Methyl deoxyCytidine (5-methyl dC), or an inverted dT.
Paragraph 9. The sensor of any one of paragraphs 6-8, wherein the bridging moiety is a locked nucleic acid.
Paragraph 10. The sensor of any one of paragraphs 2-9, wherein the terminal modification is a 5′ terminal modification phosphate modification, a 5′-phosphorylation, or a 3′-phosphorylation.
Paragraph 11. The sensor of any one of paragraphs 1-10, wherein each internucleotide linkage is a phosphorothioate linkage.
Paragraph 12. The sensor of any one of paragraphs 1-11, wherein the modified nucleic acid barcode is single-stranded or double-stranded.
Paragraph 13. The sensor of any one of paragraphs 1-12, wherein the nucleic acid barcode is at least 5, at least 10, at least 15, at least 20, at least 25, at least 30, at least 35, at least 40, at least 45, or at least 50 nucleotides in length.
Paragraph 14. The sensor of any one of paragraphs 1-13, wherein the nucleic acid barcode is between 5-30, 10-30, 15-30, 20-30, or 10-50 nucleotides in length.
Paragraph 15. The sensor of paragraph 14, wherein the nucleic acid barcode is 20 nucleotides in length.
Paragraph 16. The sensor of any one of paragraphs 1-15, wherein the modified nucleic acid barcode comprises a deoxyribonucleotide and/or a ribonucleotide.
Paragraph 17. The sensor of any one of paragraphs 1-16, wherein the modified nucleic acid barcode comprises single-stranded deoxyribonucleotides.
Paragraph 18. The sensor of any one of paragraphs 1-17, wherein the modified nucleic acid barcode comprises the nucleic acid sequence of any one of SEQ ID NOs: 15-49, or a sequence having no more than 1, 2, 3, 4, or 5 positions of difference relative thereto.
Paragraph 19. The sensor of any one of paragraphs 1-17, wherein the modified nucleic acid barcode comprises the nucleic acid sequence and modifications of: any one of SEQ ID NOs: 16, 19-27, or 35-49; a sequence having no more than 1, 2, 3, 4, or 5 positions of difference relative thereto; or a sequence having no more than 1, 2, 3, 4, or 5 positions of difference relative thereto and no more than 1, 2, 3, 4, 5, 6, 7, 8, 9, or 10 differences in modification relative thereto.
Paragraph 20. The sensor of any one of paragraphs 1-19, wherein the modified nucleic acid barcode is capable of activating the single-stranded nucleic acid cleavage activity of a Cas protein in the presence of a CRISPR RNA sequence (crRNA).
Paragraph 21. The sensor of paragraph 20, wherein the modified nucleic acid barcode comprises a sequence that is complementary to a sequence in the crRNA.
Paragraph 22. The sensor of paragraph 21, wherein the crRNA comprises a nucleic acid sequence selected from any of SEQ ID NOs: 9-14, or a sequence with no more than 1, 2, 3, 4, or 5 positions of difference relative thereto.
Paragraph 23. The sensor of any of paragraphs 20-22, wherein the Cas protein is a type V Cas protein, a type VI Cas protein, a Cas14, a CasX, a CasZ, or a CasY, optionally wherein the type VI Cas protein is Cas 13a or Cas 13b.
Paragraph 24. The sensor of paragraph 23, wherein the Cas protein is Cas12a.
Paragraph 25. The sensor of paragraph 24, wherein the Cas protein comprises an amino acid sequence of SEQ ID NO: 73 or a sequence with at least 80, 85, 90, 95, or 99% identity thereto.
Paragraph 26. The sensor of any one of paragraphs 1-25, wherein the scaffold is an antibody.
Paragraph 27. The sensor of paragraph 26, wherein the antibody is a nanobody.
Paragraph 28. The sensor of paragraph 27, wherein the scaffold comprises an amino acid sequence of either of SEQ ID NOs: 71 or 72, or a sequence with at least 80, 85, 90, 95, or 99% identity thereto.
Paragraph 29. The sensor of any one of paragraphs 1-28, wherein the sensor is linked to the modified nucleic acid barcode through a linker.
Paragraph 30. The sensor of paragraph 29, wherein the linker comprises an enzyme substrate.
Paragraph 31. The sensor of paragraph 30, wherein the enzyme substrate is capable of being cleaved by an enzyme that is dysregulated in cancer.
Paragraph 32. The sensor of either of paragraphs 30 or 31, wherein the enzyme substrate comprises a peptide comprising an amino acid sequence selected from SEQ ID NOs: 50-70, or a sequence having no more than 1, 2, 3, 4, or 5 positions of difference relative thereto.
Paragraph 33. The sensor of either of paragraphs 30 or 31, wherein the enzyme substrate comprises an enzyme-cleavable sequence comprised within an amino acid sequence selected from SEQ ID NOs: 50-70, or a sequence having no more than 1, 2, 3, 4, or 5 positions of difference relative thereto.
Paragraph 34. The sensor of any of paragraphs 30-33, wherein the enzyme substrate comprises a peptide comprising an amino acid sequence and modifications selected from SEQ ID NOs: 50-70 or a sequence having no more than 1, 2, 3, 4, or 5 positions of difference relative thereto.
Paragraph 35. The sensor of either of paragraphs 30 or 31, wherein the enzyme substrate comprises a peptide comprising an amino acid sequence selected from SEQ ID NOs: 50-54, or a sequence having no more than 1, 2, 3, 4, or 5 positions of difference relative thereto.
Paragraph 36. The sensor of either of paragraphs 30 or 31, wherein the enzyme substrate comprises an enzyme-cleavable sequence comprised within an amino acid sequence selected from SEQ ID NOs: 50-54, or a sequence having no more than 1, 2, 3, 4, or 5 positions of difference relative thereto.
Paragraph 37. The sensor of any of paragraphs 30, 31, 35, or 36, wherein the enzyme substrate comprises a peptide comprising an amino acid sequence and modifications selected from SEQ ID NOs: 50-54 or a sequence having no more than 1, 2, 3, 4, or 5 positions of difference relative thereto.
Paragraph 38. The sensor of any one of paragraphs 30-37, wherein the enzyme is a protease. Paragraph 39. The sensor of any one of paragraphs 31-38, wherein the cancer is colon cancer, liver cancer, breast cancer, lung cancer, or melanoma.
Paragraph 40. The sensor of any one of paragraphs 29-39, wherein the linker is an environmentally-responsive linker.
Paragraph 41. The sensor of paragraph 40, wherein the environmentally-responsive linker comprises a cleavable linker.
Paragraph 42. The sensor of any one of paragraphs 1-41 comprising a plurality of cleavable linkers.
Paragraph 43. The sensor of any one of paragraphs 1-42 comprising a plurality of modified nucleic acid barcodes.
Paragraph 44. The sensor of paragraph 40-43, wherein each modified nucleic acid barcode uniquely identifies an environmentally-responsive linker.
Paragraph 45. The sensor of any one of paragraphs 29-44, wherein the linker comprises a rigid linker.
Paragraph 46. The sensor of paragraph 45, wherein the rigid linker comprises the sequence SPSTPPTPSPSTPP (SEQ ID NO: 6).
Paragraph 47. The sensor of any of paragraphs 1-46, wherein the modified nucleic acid barcode has a molecular weight of 3-20, 3-15, 3-10, 3-8, 3-5, 5-20, 5-15, 5-10, 5-8, 8-20, 8-15, 8-10, 10-20, 10-15, or 15-20 kilodaltons (kDa).
Paragraph 48. A method of detecting an enzyme that is active in a subject comprising:
a) obtaining a sample from a subject who has been administered the sensor of any one of paragraphs 1-47; and
b) detecting the modified nucleic acid barcode, wherein detection of the modified nucleic acid is indicative of the enzyme being in the active form in the subject.
   Paragraph 49. The method of paragraph 48, wherein detecting the modified nucleic acid barcode comprises contacting the sample with a Cas-based nucleic acid detection system that comprises:
(i) a crRNA sequence that comprises a guide sequence that is complementary to a sequence in the modified nucleic acid barcode;
(ii) a Cas protein; and
(iii) a reporter that comprises a first ligand that is connected to a second ligand through a single-stranded nucleic acid linker, wherein the single-stranded nucleic acid linker is not complementary to the guide sequence; and
detecting cleavage of the reporter.
   Paragraph 50. The method of paragraph 49, wherein:

a) the reporter is a fluorescently quenched reporter and detecting cleavage of the reporter comprises detecting an increase in fluorescence as compared to the level of fluorescence detected in the system in the absence of the sample from the subject; or

b) the first ligand binds a different antibody as compared to the second ligand and detecting cleavage of the reporter comprises using a lateral flow assay.

Paragraph 51. The method of any one of paragraphs 48-50, wherein cleavage of the reporter is detected in less than 5 hours, less than 4 hours, at least 3 hours, less than 2 hours, or less than 1 hour following contacting the sample with the system.
Paragraph 52. The method of any of paragraphs 48-51, wherein the crRNA comprises a nucleic acid sequence selected from any of SEQ ID NOs: 9-14, or a sequence with no more than 1, 2, 3, 4, or 5 positions of difference relative thereto.
Paragraph 53. The method of any of paragraphs 49-52, wherein the Cas protein is Cas12a.
Paragraph 54. The method of any of paragraphs 49-53, wherein the Cas protein comprises an amino acid sequence of SEQ ID NO: 73 or a sequence with at least 80, 85, 90, 95, or 99% identity thereto.
Paragraph 55. An article comprising a housing comprising a membrane having:

a) a defined region with a detection reagent bound thereto;

b) a reservoir capable of housing a biological sample from a subject who has been administered a sensor of any one of paragraphs 1-47 in contact with the membrane such that the biological sample can be delivered to the reservoir comprising a Cas-based nucleic acid detection system that comprises:
i) a crRNA sequence that comprises a guide sequence that is complementary to a sequence in the modified nucleic acid barcode;
ii) a Cas protein; and
iii) a reporter that comprises a first ligand that is connected to a second ligand through a single-stranded nucleic acid linker, wherein the single-stranded nucleic acid linker is not complementary to the guide sequence; and such that the biological sample can move along the membrane,
c) a conjugate pad on the membrane, wherein an affinity agent for binding to a capture ligand is associated with the conjugate pad, wherein the detection reagent detects the first ligand on the reporter and the affinity agent detects the second ligand on the reporter.
   Paragraph 56. The article of paragraph 55, wherein the membrane is a nitrocellulose membrane.
   Paragraph 57. The article of paragraph 55, wherein the affinity agent is streptavidin bound to gold nanoparticles.
   Paragraph 58. The article of paragraph 55, wherein the capture ligand is biotin.
   Paragraph 59. The article of paragraph 55, wherein the reservoir is a cellulose pad.
   Paragraph 60. The article of paragraph 55, wherein the detection reagent is an antibody specific for the second ligand.
   Paragraph 61. The article of paragraph 55, wherein the antibody is a a-FAM antibody.
   Paragraph 62. The article of any one of paragraphs 55-61, wherein the biological sample is a urine sample, saliva sample, fecal sample, seminal fluid sample, or a cerebrospinal fluid sample.
   Paragraph 63. The article of any of paragraphs 55-62, wherein the crRNA comprises a nucleic acid sequence selected from any of SEQ ID NOs: 9-14, or a sequence with no more than 1, 2, 3, 4, or 5 positions of difference relative thereto.
   Paragraph 64. The article of any of paragraphs 55-63, wherein the Cas protein is Cas12a.
   Paragraph 65. The article of any of paragraphs 55-64, wherein the Cas protein comprises an amino acid sequence of SEQ ID NO: 73 or a sequence with at least 80, 85, 90, 95, or 99% identity thereto.
   Paragraph 66. A composition comprising:

a first sensor of any of paragraphs 1-47 comprising a first barcode, and

a second sensor of any of paragraphs 1-47 comprising a second barcode,

wherein the barcode of the first sensor is different from the barcode of the second sensor, and

wherein the enzyme capable of releasing the barcode from the first sensor is different from the enzyme capable of releasing the barcode from the second sensor.

Paragraph 67. A composition comprising:

a first sensor comprising a first modified nucleic acid barcode that is capable of being released from the sensor when exposed to a first enzyme present in a subject, and

a second sensor comprising a second modified nucleic acid barcode that is capable of being released from the sensor when exposed to a second enzyme present in a subject,

wherein the first sensor and second sensor are linked to a scaffold,

wherein the barcode of the first sensor is different from the barcode of the second sensor, and

wherein the enzyme capable of releasing the barcode from the first sensor is different from the enzyme capable of releasing the barcode from the second sensor.

Paragraph 68. The composition of either of paragraphs 66 or 67, further comprising a third sensor comprising a barcode that is different from both the barcode of the first sensor and the barcode of the second sensor, and wherein the enzyme capable of releasing the barcode from the third sensor is different from the enzymes capable of releasing the barcodes from the first and second sensors.
Paragraph 69. The composition of paragraph 68, wherein the third sensor is a sensor of any of paragraphs 1-47.
Paragraph 70. The composition of paragraph 68, wherein the third sensor is linked to the scaffold.
Paragraph 71. A method of diagnosing a subject with a disease associated with the activity of an enzyme, the method comprising:
a) obtaining a sample from a subject who has been administered the sensor of any one of paragraphs 1-47;
b) detecting the modified nucleic acid barcode, wherein the presence of the modified nucleic acid is indicative of the enzyme being in the active form in the subject; and
c) responsive to (b), diagnosing the subject with the disease associated with the activity of the enzyme.
   Paragraph 72. A method of diagnosing a subject with a disease associated with an activity profile, the method comprising:
a) obtaining a sample from a subject who has been administered a plurality of the sensors of any one of paragraphs 1-47 or the composition of any one of paragraphs 66-70;
b) detecting one or more modified nucleic acid barcodes from the sensors, wherein the presence of a modified nucleic acid is indicative of the corresponding enzyme being in the active form in the subject, thereby determining an activity profile for the subject; and
c) responsive to the activity profile, diagnosing the subject with the disease associated with the activity profile.
   Paragraph 73. The method of any of paragraphs 71 or 72, wherein detecting the modified nucleic acid barcode comprises contacting the sample with a Cas-based nucleic acid detection system that comprises:
(i) a crRNA sequence that comprises a guide sequence that is complementary to a sequence in the modified nucleic acid barcode;
(ii) a Cas protein; and
(iii) a reporter that comprises a first ligand that is connected to a second ligand through a single-stranded nucleic acid linker, wherein the single-stranded nucleic acid linker is not complementary to the guide sequence; and
detecting cleavage of the reporter.
   Paragraph 74. The method of paragraph 73, wherein:

a) the reporter is a fluorescently quenched reporter and detecting cleavage of the reporter comprises detecting an increase in fluorescence as compared to the level of fluorescence detected in the system in the absence of the sample from the subject; or

b) the first ligand binds a different antibody as compared to the second ligand and detecting cleavage of the reporter comprises using a lateral flow assay.

Paragraph 75. The method of any of paragraphs 73-74, wherein the crRNA comprises a nucleic acid sequence selected from any of SEQ ID NOs: 9-14, or a sequence with no more than 1, 2, 3, 4, or 5 positions of difference relative thereto.
Paragraph 76. The method of any of paragraphs 73-75, wherein the Cas protein is Cas12a.
Paragraph 77. The method of any of paragraphs 73-76, wherein the Cas protein comprises an amino acid sequence of SEQ ID NO: 73 or a sequence with at least 80, 85, 90, 95, or 99% identity thereto.

1. A sensor comprising a scaffold linked to a modified nucleic acid barcode that is capable of being released from the sensor when exposed to an enzyme present in a subject.

2. The sensor of claim 1, wherein the modified nucleic acid barcode comprises a modified internucleoside linkage, a modified nucleotide, and/or a terminal modification.

3. The sensor of claim 2, wherein the modified internucleoside linkage is selected from a phosphorothioate linkage or a boranophosphate linkage.

4. (canceled)

5. The sensor of claim 1, wherein the modified nucleic acid barcode comprises a modified sugar moiety and/or a modified base.

6. The sensor of claim 5, wherein the modified sugar moiety comprises a 2′-OH group modification and/or a bridging moiety.

7. The sensor of claim 6, wherein the 2′-OH group modification is selected from the group consisting of 2′-O-Methyl (2′-O-Me), 2′-Fluoro (2′-F), and 2′-O-methoxy-ethyl (2′-O-MOE).

8. The sensor of claim 5, wherein the modified base is a deoxyuridine (dU), a 5-Methyl deoxyCytidine (5-methyl dC), or an inverted dT.

9. (canceled)

10. The sensor of claim 2, wherein the terminal modification is a 5′ terminal modification phosphate modification, a 5′-phosphorylation, or a 3′-phosphorylation.

11. (canceled)

12. The sensor of claim 1, wherein the modified nucleic acid barcode is single-stranded or double-stranded.

13. The sensor of claim 1, wherein the modified nucleic acid barcode is 20 nucleotides in length.

14. The sensor of claim 1, wherein the modified nucleic acid barcode comprises a deoxyribonucleotide and/or a ribonucleotide.

15. The sensor of claim 1, wherein the modified nucleic acid barcode is capable of activating the single-stranded nucleic acid cleavage activity of a Cas protein in the presence of a CRISPR RNA sequence (crRNA).

16. The sensor of claim 15, wherein the Cas protein is a type V Cas protein, a type VI Cas protein, a Cas14, a CasX, a CasZ, or a CasY, optionally wherein the type VI Cas protein is Cas 13a or Cas 13b.

17. The sensor of claim 1, wherein the scaffold is an antibody.

18. The sensor of claim 1, wherein the modified nucleic acid barcode comprises a sequence selected from SEQ ID NOs: 16, 19-27, or 35-49 or a sequence from Table 11.

19. The sensor of claim 1, wherein the modified nucleic acid is linked to an enzyme-cleavable substrate that is linked to the scaffold.

20. The sensor of claim 19, wherein the enzyme-cleavable substrate comprises a sequence selected from SEQ ID NOs: 50-70.

21. (canceled)

22. A method of detecting an enzyme that is active in a subject comprising:
a) obtaining a sample from a subject who has been administered the sensor of any one of claim 1; and
b) detecting the modified nucleic acid barcode, wherein detection of the modified nucleic acid is indicative of the enzyme being in the active form in the subject.

23. The method of claim 22, wherein detecting the modified nucleic acid barcode comprises contacting the sample with a system that comprises:
(i) a crRNA sequence that comprises a guide sequence that is complementary to a sequence in the modified nucleic acid barcode;
(ii) a Cas protein; and
(iii) a reporter that comprises a first ligand that is connected to a second ligand through a single-stranded nucleic acid linker, wherein the single-stranded nucleic acid linker is not complementary to the guide sequence; and
detecting cleavage of the reporter.

24. (canceled)

25. The method of claim 23, wherein the crRNA sequence comprises a sequence selected from SEQ ID NOs: 9-14 or Table 10.
